(19) United States
(12) Patent Application Publication
(10) Pub. No.: US 2025/0121055 A1
(43) Pub. Date: Apr. 17, 2025

(54) INTERFERON- INDUCING COMPLEXES AND RNA DUPLEXES AND METHODS OF USE

(71) Applicant: PRESIDENT AND FELLOWS OF HARVARD COLLEGE, Cambridge, MA (US)

(72) Inventors: Longlong SI, Cambridge, MA (US); Haiqing BAI, Cambridge, MA (US); Crystal Yuri OH, Cambridge, MA (US); Donald E. INGBER, Cambridge, MA (US)

(73) Assignee: PRESIDENT AND FELLOWS OF HARVARD COLLEGE, Cambridge, MA (US)

(21) Appl. No.: 18/685,473

(22) PCT Filed: Aug. 30, 2022
(86) PCT No.: PCT/US2022/042006

§ 371 Date: Feb. 21, 2024

Related U.S. Application Data
(60) Provisional application No. 63/238,929, filed on Aug. 31, 2021.
(60) Provisional application No. 63/392,660, filed on Jul. 27, 2022.

(51) Int. Cl. A61K 39/39; A61K 39/00; A61K 39/145; A61K 39/215; A61K 45/06; A61P 37/04; C07H 21/02

(52) U.S. Cl. CPC A61K 39/39; A61K 39/145; A61K 39/215; A61K 45/06; A61P 37/04; C07H 21/02; A61K 2039/53; A61K 2039/544

(57) ABSTRACT

Pathogenic infections trigger a complex regulatory system of innate and adaptive immune responses designed to defend against the pathogen in the host organism. One of the many responses to pathogen invasion, e.g., viral, bacterial, fungal or parasitic infection, is the induction of interferon (IFN) production, a pleiotropic group of cytokines that play a critical role in human immune responses by ‘interfering’ with pathogen activity, e.g., viral replication, among others. Described herein are compositions and methods for inducing Type I interferon production. The compositions described comprise immunostimulatory complexes and RNA duplexes. Compositions comprising the immunostimulatory complexes and RNA duplexes described can be used for the treatment of diseases or disorders that respond to interferons.

### CROSS-REFERENCE TO RELATED APPLICATIONS

This application is a 35 U.S.C. § 371 National Phase Entry application of International Application No. PCT/US2022/042006 filed Aug. 30, 2022, which designates the U.S., and which claims benefit under 35 U.S.C. § 119(e) of U.S. Provisional Application No. 63/238,929 filed Aug. 31, 2021 and U.S. Provisional Application No. 63/392,660 filed Jul. 27, 2022, contents of which are incorporated by reference in their entireties.

### GOVERNMENT SUPPORT

This invention was made with government support under HL141797 awarded by the National Institutes of Health and under HR0011-19-2-0008 and HR0011-20-2-0040 awarded by the U.S. Department of Defense/DARPA. The government has certain rights in the invention.

### SEQUENCE LISTING

The instant application contains a Sequence Listing which has been submitted electronically in XML format and is hereby incorporated by reference in its entirety. Said XML copy, created on Oct. 10, 2022, is named 002806-190320WOPT_SL.xml and is 55,841 bytes in size.

### TECHNICAL FIELD

The technology described herein relates to compositions and methods for immunostimulation.

### BACKGROUND

Pathogenic infections trigger a complex regulatory system of innate and adaptive immune responses designed to defend against the pathogen in the host organism. One of the many responses to pathogen invasion, e.g., viral, bacterial, fungal or parasitic infection, is the induction of interferon (IFN) production, a pleiotropic group of cytokines that play a critical role in human immune responses by ‘interfering’ with pathogen activity, e.g., viral replication, among others. The increasing incidence of pandemic viruses, such as influenza, MERS, SARS, and now SARS-CoV-2, requires development of new broad-spectrum therapies that inhibit infection by many different types of viruses and pathogens.

### SUMMARY

One aspect described herein provides an immunostimulatory complex comprising a concatamer of oligonucleotide duplexes, wherein each duplex comprises an oligonucleotide strand having the structure 5′-C—N₁₆-GGG-3′ and an oligonucleotide strand having the structure 5′-C—N′₁₆-GGG-3′, wherein: N and N′ are each any of G, A, U and C; N₁₆ is complementary to N′₁₆; and duplexes in the concatamer are joined by Hoogsteen base pairing between 3′-GG overhanging dinucleotides on each duplex.

In one embodiment of any aspect described herein, the concatamer is a dimer of oligonucleotide duplexes.

In one embodiment of any aspect described herein, the concatamer comprises three or more of the oligonucleotide duplexes.

In one embodiment of any aspect described herein, one or both oligonucleotide strands of each duplex comprise(s) a 5′-terminal monophosphate, diphosphate, triphosphate or hydroxyl group.

In one embodiment of any aspect described herein, the oligonucleotide duplexes comprise double stranded RNA.

In one embodiment of any aspect described herein, the concatamer induces interferon (IFN) production in a cell.

In one embodiment of any aspect described herein, the IFN production is type I IFN production.

In one embodiment of any aspect described herein, the concatamer activates the RIG-I-IRF3 pathway.

In one embodiment of any aspect described herein, the concatamer reduces a viral titer or viral load in a cell or population of cells.

Another aspect described herein provides an immunostimulatory complex comprising at least first and second RNA duplexes, each duplex comprising: a first strand comprising, from the 5′ terminus, the sequence 5′-C—N₁₉-3′, and a second strand comprising, at the 3′ terminus, the sequence 5′-N′₁₉-GGG-3′, wherein: N and N′ are any of C, A, G, and U; N and N′ are complementary; the 3′ terminal GG dinucleotide of the second strand forms a 3′ GG dinucleotide overhang; the first duplex is complexed with the at least second duplex via Hoogsteen base pairing between the 3′ GG overhang on each duplex; and the first strand, at the 5′ terminus, does not comprise the sequence 5′-CUGA-3′.

In one embodiment of any aspect described herein, one or both oligonucleotide strands of each duplex comprise(s) a 5′-terminal monophosphate, diphosphate, triphosphate or hydroxyl group.

In one embodiment of any aspect described herein, the RNA duplexes comprise double stranded RNA.

In one embodiment of any aspect described herein, the RNA duplexes comprise one or more DNA nucleotides at the duplex end opposite the 5′-C.

In one embodiment of any aspect described herein, the RNA duplexes comprise comprises a blunt end, a 5′ overhang or a 3′ overhang on the end opposite the 5′-C.

In one embodiment of any aspect described herein, the complex induces interferon (IFN) production in a cell.

In one embodiment of any aspect described herein, the IFN production is type I IFN production.

In one embodiment of any aspect described herein, the complex activates the RIG-I-IRF3 pathway.

In one embodiment of any aspect described herein, the complex reduces a viral titer or viral load in a cell or population of cells.

Another aspect described herein provides a pharmaceutical composition comprising any of the immunostimulatory complexes described herein.

In one embodiment of any aspect described herein, the composition further comprises a pharmaceutically acceptable carrier.

In one embodiment of any aspect described herein, the composition is formulated for airway administration.

In one embodiment of any aspect described herein, the composition is formulated for aerosol administration, nebulizer administration, or tracheal lavage administration.

Another aspect described herein provides a composition comprising any of the immunostimulatory complexes described herein or any of the pharmaceutical compositions described herein and a vaccine.

Another aspect described herein provides a composition comprising any of the immunostimulatory complexes described herein or any of the pharmaceutical compositions described here and a nanoparticle.

Another aspect described herein provides a nanoparticle comprising any of the immunostimulatory complexes described herein or any of the pharmaceutical compositions described herein.

Another aspect described herein provides a method of inducing an anti-viral response in a subject, the method comprising administering to a subject in need thereof any of the immunostimulatory complexes described herein or any of the pharmaceutical compositions described herein.

Another aspect described herein provides a method of treating or preventing a viral infection in a subject, the method comprising administering to a subject in need thereof any of the immunostimulatory complexes described herein or any of the pharmaceutical compositions described herein.

In one embodiment of any aspect described herein, the subject in need thereof has a viral infection, or is at risk of having a viral infection.

In one embodiment of any aspect described herein, the method further comprises, prior to administering, a step of diagnosing the subject as having a viral infection or being at risk of having a viral infection.

In one embodiment of any aspect described herein, the method further comprises, prior to administering, a step of receiving results of an assay that diagnoses the subject as having a viral infection or as being at risk of having a viral infection.

In one embodiment of any aspect described herein, the viral infection is caused by a virus selected from the group consisting of: John Cunningham virus, measles virus, Lymphocytic choriomeningitis virus, arbovirus, rabies virus, rhinovirus, parainfluenza virus, respiratory syncytial virus, herpes simplex virus, herpes simplex type 1, herpes simplex type 2, human herpesvirus 6, adenovirus, cytomegalovirus, Epstein-Barr virus, mumps virus, influenza virus type A, influenza virus type B, coronavirus, SARS coronavirus, SARS-CoV-2 virus, coxsackie A virus, coxsackie B virus, poliovirus, HTLV-1, hepatitis virus types A, B, C, D, and E, varicella zoster virus, smallpox virus, molluscum contagiosum, human papillomavirus, parvovirus B19, rubella virus, human immunodeficiency virus, rotavirus, norovirus, astrovirus, ebola virus, Marburg virus, dengue virus (DENV), and Zika virus.

In one embodiment of any aspect described herein, the viral infection is an infection of a tissue selected from the group consisting of central nervous system tissue, eye tissue, upper respiratory system tissue, lower respiratory system tissue, lung tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.

In one embodiment of any aspect described herein, the administration is systemic.

In one embodiment of any aspect described herein, the administration is local at a site of viral infection.

In one embodiment of any aspect described herein, the method further comprises administering at least one additional therapeutic.

In one embodiment of any aspect described herein, the at least one additional therapeutic is an anti-viral therapeutic.

Another aspect described herein provides a method of treating an influenza infection in a subject, the method comprising administering to a subject having an influenza infection any of the immunostimulatory complexes described herein or any of the pharmaceutical compositions described herein.

In one embodiment of any aspect described herein, the influenza infection is an influenza A infection, or an influenza B infection.

In one embodiment of any aspect described herein, the method further comprises administering at least one additional anti-viral therapeutic.

Another aspect described herein provides a method of treating a coronavirus disease in a subject, the method comprising administering to a subject having a coronavirus infection any of the immunostimulatory complexes described herein or any pharmaceutical composition described here.

In one embodiment of any aspect described herein, the coronavirus disease is COVID-19.

In one embodiment of any aspect herein, the method further comprises administering at least one additional anti-viral therapeutic.

Another aspect described herein provides a method of inducing interferon (IFN) production, the method comprising administering to a subject in need thereof any of the immunostimulatory complexes described herein or any pharmaceutical composition described herein, whereby IFN production is increased following administration.

In one embodiment of any aspect described herein, IFN production is the production of type I IFN, type II IFN, or type III IFN.

In one embodiment of any aspect described herein, IFN production is the production of type I IFN.

Another aspect described herein provides an immunostimulatory RNA duplex having a) a first strand having from 5′ to 3′ a GNNN sequence flanked by at least 22 nucleotides on each side; and b) a second strand having from 5′-3′ a GGGC sequence flanked by at least 22 nucleotides on each side, wherein the first and second strands are complementary to each other.

In one embodiment of any aspect described herein, the first and/or second strand has a two nucleotide overhang at its 3′ end.

In one embodiment of any aspect described herein, the first and/or second strand have two DNA nucleosides at its 3′ end.

In one embodiment of any aspect described herein, the DNA nucleosides are thymidines.

In one embodiment of any aspect described herein, the first and/or second strand has a TT overhang at its 3′ end.

In one embodiment of any aspect described herein, the first and/or second strand comprises a 5′-terminal monophosphate, diphosphate, triphosphate or hydroxyl group.

In one embodiment of any aspect described herein, the RNA duplex is synthetic.

In one embodiment of any aspect described herein, the RNA duplex induces interferon (IFN) production in a cell.

In one embodiment of any aspect described herein, the IFN production is type I IFN production.

In one embodiment of any aspect described herein, the RNA duplex activates the RIG-I-IRF3 pathway.

In one embodiment of any aspect described herein, the RNA duplex reduces a viral titer or viral load in a cell or population of cells.

Another aspect described herein provides a synthetic RNA duplex having a first and second strand having a sequence selected from SEQ ID NO: 5-30.

Another aspect described herein provides a method of inducing an anti-viral response is a subject, the method comprising administering to a subject in need thereof any of the RNA duplexes described herein.

Another aspect described herein provides a method of treating a viral infection in a subject, the method comprising administering to a subject in need thereof any of the RNA duplexes described herein.

In one embodiment of any aspect described herein, the subject in need thereof has a viral infection, or is at risk of having a viral infection.

In one embodiment of any aspect described herein, the method further comprises, prior to administering, a step of diagnosing the subject as having a viral infection or being at risk of having a viral infection.

In one embodiment of any aspect described herein, the method further comprises, prior to administering, a step of receiving results of an assay that diagnoses the subject as having a viral infection or as being at risk of having a viral infection.

In one embodiment of any aspect described herein, the viral infection is caused by a virus selected from the group consisting of: John Cunningham virus, measles virus, Lymphocytic choriomeningitis virus, arbovirus, rabies virus, rhinovirus, parainfluenza virus, respiratory syncytial virus, herpes simplex virus, herpes simplex type 1, herpes simplex type 2, human herpesvirus 6, adenovirus, cytomegalovirus, Epstein-Barr virus, mumps virus, influenza virus type A, influenza virus type B, coronavirus, SARS coronavirus, SARS-CoV-2 virus, coxsackie A virus, coxsackie B virus, poliovirus, HTLV-1, hepatitis virus types A, B, C, D, and E, varicella zoster virus, smallpox virus, molluscum contagiosum, human papillomavirus, parvovirus B19, rubella virus, human immunodeficiency virus, rotavirus, norovirus, astrovirus, ebola virus, Marburg virus, dengue virus (DENV), and Zika virus.

In one embodiment of any aspect described herein, the viral infection is an infection of a tissue selected from the group consisting of central nervous system tissue, eye tissue, upper respiratory system tissue, lower respiratory system tissue, lung tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.

In one embodiment of any aspect described herein, the administration is systemic.

In one embodiment of any aspect described herein, the administration is local at a site of viral infection.

In one embodiment of any aspect described herein, the method further comprises administering at least one additional therapeutic.

In one embodiment of any aspect described herein, the at least one additional therapeutic is an anti-viral therapeutic.

Another aspect described herein provides a method of treating an influenza infection in a subject, the method comprising administering to a subject having an influenza infection any of the RNA duplexes described herein.

In one embodiment of any aspect described herein, the influenza infection is an influenza A infection, or an influenza B infection.

In one embodiment of any aspect described herein, the method further comprises administering at least one additional anti-viral therapeutic.

Another aspect described herein provides a method of treating a coronavirus disease in a subject, the method comprising administering to a subject having a coronavirus disease any ofthe RNA duplexes described herein.

In one embodiment of any aspect described herein, the coronavirus disease is COVID-19.

In one embodiment of any aspect described herein, the method further comprises administering at least one additional anti-viral therapeutic.

Another aspect described herein provides a method of increasing the efficacy of an anti-viral therapeutic, the method comprising administering any RNA duplex described herein and at least one anti-viral therapeutic.

In one embodiment of any aspect described herein, the anti-viral therapeutic is selected from the group consisting of: Abacavir, Acyclovir (Aciclovir), Adefovir, Amantadine, Ampligen, Amprenavir (Agenerase), Amodiaquine, Apilimod, Arbidol, Atazanavir, Atripla, Atovaquone, Balavir, Baloxavir marboxil (Xofluza®), Biktarvy Boceprevir (Victrelis®), Cidofovir, Clofazimine, Clomifene, Clofazamine, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Favipiravir, Fenofibrate, Fomivirsen, Fosamprenavir, Foscamet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Ivermectin, Lamivudine, Lasalocid, Letermovir (Prevymis®), Lopinavir, Loviride, Mannose Binding Lectin, Maraviroc, Methisazone, Moroxydine, Nafamostat, Nelfinavir, Nevirapine, Nexavir®, Nilotinib, Nitazoxanide, Norvir, Nucleoside analogues, Oseltamivir (Tamiflu®), Pazopanib, Peginterferon alfa-2a, Peginterferon alfa-2b, Penciclovir, Peramivir (Rapivab®), Pleconaril, Podophyllotoxin, Protease inhibitor (pharmacology), Pyonaridine, Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Tafenoquine, Telaprevir, Telbivudine (Tyzeka®), Tenofovir alafenamide, Tenofovir disoproxil, Tenofovir, Toremifene, Tipranavir, Trifluridine, Trizivir, Tromantadine, Truvada, Valaciclovir (Valtrex), Valganciclovir, Vermurafenib, Venetoclax, Vicriviroc, Vidarabine, Viramidine, Zalcitabine, Zanamivir (Relenza®), and Zidovudine.

In one embodiment of any aspect described herein, the RNA duplex and the at least one antiviral therapeutic are administered at substantially the same time.

In one embodiment of any aspect described herein, the RNA duplex and the at least one antiviral therapeutic are administered at different time points.

Another aspect described herein provides a pharmaceutical composition comprising any RNA duplex described herein and a pharmaceutically acceptable carrier.

Another aspect described herein provides a pharmaceutical composition comprising any RNA duplex described herein and at least one anti-viral therapeutic.

In one embodiment of any aspect described herein, the composition is formulated for airway administration.

In one embodiment of any aspect described herein, the composition is formulated for aerosol administration, nebulizer administration, or tracheal lavage administration.

Another aspect described herein provides a method of inducing interferon (IFN) production, the method comprising administering to a subject in need thereof any RNA duplex described herein, or any pharmaceutical composition described herein, whereby IFN production is increased following administration.

In one embodiment of any aspect described herein, IFN production is the production of type I IFN, type II IFN, or type III IFN.

In one embodiment of any aspect described herein, IFN production is the production of type I IFN.

In one embodiment of any aspect described herein, the type I IFN is IFN-α, IFN-β, IFN-ε, IFN-κ or IFN-ω.

In one embodiment of any aspect described herein, increased IFN production increases cellular resistance to a viral infection.

Another aspect described herein provides a composition any RNA duplex described herein and a vaccine.

Another aspect described herein provides a composition comprising any RNA duplex described herein and a nanoparticle.

Another aspect described herein provides a method of vaccinating, the method comprising administering to a subject in need thereof any composition described herein.

Another aspect described herein provides a method of increasing the efficacy of a vaccine, the method comprising administering to a subject in need thereof any immunostimulatory complex described herein, any composition described herein, or any RNA duplex described herein.

Another aspect described herein provides a method of preparing an RNAi molecule to promote degradation of a target RNA, the method comprising: a) identifying CCC trinucleotide repeats in the sequence of a target RNA; b) selecting a nucleotide sequence from 20 nucleotides to the upper limit for a dsRNA duplex that avoids a double-stranded RNA-activated protein kinase response, and lacks CCC repeats in a target RNA sequence as a candidate RNAi sequence (see, e.g., Lemaire et al., J. Mol. Biol. 381: 351-360 (2008), which teaches that 30 bp is the smallest dsRNA that elicits PKR activity; thus, in one embodiment, the nucleotide sequence selected can be 20-29 nucleotides, e.g., 20-28, 20-27, 20-26, 20-25, 20-24, 20-23, 20-22 or 20-21 nucleotides in length); c) synthesizing an RNA molecule complementary to the sequence selected in step (b); and d) synthesizing an RNA molecule complementary to the RNA molecule synthesized in step (c), wherein combination of the RNA molecules synthesized in steps (c) and (d) produces an RNAi molecule that is less immunostimulatory than an RNAi molecule that targets the same target RNA but comprises a CCC trinucleotide repeat.

### Definitions

As used herein, an “RNA duplex” encompasses two separate strands of ribonucleic acid that hybridize through the formation of complementary base pairs to form a duplex under physiologically relevant conditions of temperature and ionic strength. As used herein, the term “oligonucleotide duplex” encompasses a single strand that includes self-complementary sequences that permit base pairing to form an intramolecular duplex under similar conditions. Duplexes formed from a single strand can include a hairpin structure that folds back on itself with few non-hybridized nucleotides at the transition from one strand of the duplex to the other, or a hairpin loop or stem loop structure that includes a more pronounced loop of non-hybridized nucleotides between the hybridized sequences. In some embodiments, an RNA duplex can include a single-stranded overhang. In some embodiments, an RNA duplex as described herein can include a minor component, generally less than 15% (e.g., less than 10%, less than 5% or fewer) of DNA nucleotides; in these instances, the DNA nucleotides will most often be, for example, at the end of a duplex strand, but can be located elsewhere as long as specified RNA nucleotides are maintained. In some embodiments, one or more DNA nucleotides can form an overhang on a duplex.

As used herein, the term “immunostimulatory complex” refers to a nucleic acid structure that promotes an antimicrobial or antiviral response by the innate immune system, including but not limited to an interferon response. The term “immunostimulatory complex” as used herein encompasses RNA duplexes as described herein, as well as concatamer of RNA oligonucleotide duplexes. Immunostimulatory complexes as described herein will have a duplexed length of at least 18 nucleotides or more, not including single stranded overhang (generally GG or a modified form thereof). While a minimum length of 18 nucleotides of duplexed sequence has been determined for immunostimulatory activity of the oligonucleotide duplexes described herein, it is contemplated that some degree of mismatch can be tolerated, e.g., within N16/N′16 sequences comprised by some embodiments of the complexes described herein, such that, for example, at least 11 of the 16 nucleotides must be complementary, e.g., at least 11 of the 16, at least 12 of the 16, at least 13 of the 16, at least 14 of the 16, at least 15 of the 16 or all of the at least 16 nucleotides are complementary. Where there is one or more mismatch, it is anticipated that mismatch(es) will be better tolerated if located in the interior of the N16/N16′ nucleotide sequence that forms a duplex—i.e., a stretch of nucleotides at both ends are fully complementary, and it is also anticipated that where there are more than one mismatch within the sequence, contiguous mismatches may be less favorable. It is also contemplated that where there is one or more mismatch, a relatively higher GC content in the remaining nucleotides may help offset any relative disadvantage of the mismatch. The same principles would apply for mismatches where the duplex region is greater than 48 nucleotides in length.

As used herein, the term “RNA” refers to ribonucleic acid, which as typically transcribed in nature comprises the purine nucleobases adenine and guanine and the pyrimidine nucleobases cytosine and uracil. RNA oligonucleotides described herein can include modified nucleobases or modifications to the ribose-phosphate backbone that, for example, enhance stability or resistance to degradation. Examples of such modifications are discussed herein below or known in the art. In one embodiment of any of the aspects described herein, the modification is not removal of the 2′ hydroxyl that distinguishes RNA from deoxyribonucleic acid.

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible, statistically significant increase in the level of such marker.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to an appropriate control.

As used herein, a “reference” or “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with interferon-mediated disease, or a biological sample that has not been contacted with a composition disclosed herein).

As used herein, an “appropriate control” refers to an untreated, otherwise identical cell or population (e.g., a patient who was not administered an agent described herein, or was administered only a subset of compositions described herein, as compared to a non-control cell).

As used herein, the term “induces interferon production” or “increases interferon production” means that interferon production is increased by at least three-fold following administration of an immunostimulatory complex or RNA duplex as described herein or following contacting of a cell, population of cells, tissue or organism with such immunostimulatory complex or RNA duplex. In some embodiments, an increase in interferon production can be at least four-fold, at least five-fold, at least 10-fold, at least 15-fold, at least 20-fold or more. Interferon production can be measured, for example, by immunoassay (e.g., ELISA, immunoprecipitation, etc.), biological reporter assay or other assays as known in the art.

As used herein, an “interferon associated disease or disorder” or a disease or disorder associated with interferon(s)” is a disease or disorder treatable by administering an interferon, or by inducing production of an interferon.

As used herein, the term “reduce a viral titer” or “reduces viral titer” means that the number of infectious viral particles in a sample, e.g., a serum, blood or tissue sample, or in a cell culture supernate, is reduced by at least 10% by treatment of a subject or a cell culture with an immunostimulatory complex or RNA duplex as described herein.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with an infection. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

As used herein “preventing” or “prevention” refers to any methodology where the disease state does not occur due to the actions of the methodology (such as, but not limited to, administration of a vaccine which prevents infection or illness due to a pathogen). In one aspect, it is understood that prevention can also mean that the disease is not established to the extent that occurs in untreated controls. Accordingly, prevention of a disease encompasses a reduction in the likelihood that a subject can develop the disease, relative to an untreated subject (e.g. a subject who is not treated with the methods or compositions described herein).

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate anon-limiting example. Thus, the abbreviation “e.g.” is synonymous with the term “for example.”

### BRIEF DESCRIPTION OF THE DRAWINGS

FIG. 1A-E show discovery of new immunostimulatory RNAs. (FIG. 1A) A549 cells were transfected with RNA-1, RNA-2, or a scrambled duplex RNA control, and infected with influenza A/WSN/33 (H1N1) virus (MOI=0.01) 24 hours later. Titers of progeny viruses in medium supernatants collected at 48 h post-infection were determined by quantifying plaque forming units (PFUs); data are shown as % viral infection measured in the cells treated with the control RNA (Data shown are mean±standard deviation; N=3; ***, p<0.001). (FIG. 1B) A549 cells were transfected with RNA-1 or a scrambled dsRNA control, collected at 48 h, and analyzed by RNA-seq (left) or TMT Mass Spec (right). Differentially expressed genes (DEGs) from RNA-seq or proteins from TMT Mass Spec are shown in volcano plots (top) and results of GO Enrichment analysis performed for the DEGs are shown at the bottom (N=3). (FIG. 1C) qPCR analysis of cellular IFN-β and IFN-α RNA levels at 48 h after A549 cells were transfected with RNA-1, RNA-2, or scrambled dsRNA control (N=3). (FIG. 1D) RNA-mediated production kinetics of IFN production in wild-type A549-Dual cells that were transfected with RNA-1, RNA-2, or scramble RNA control measured using a Quanti-Luc assay. OD values from cells transfected with the scrambled RNA control were subtracted as background (N=6). (FIG. 1E) Dose-dependent induction of IFN by RNA-1 and -2 in A549-Dual cells compared to scrambled RNA control measured at 48 h post-transfection (control OD values were subtracted as background; N=6).

FIGS. 2A and 2B show profiling the effects of RNA-2 by RNA-seq and TMT mass spectrometry. A549 cells were transfected with RNA-2 or scrambled RNA control, cell lysates were collected at 48 h, and analyzed by RNA-seq (FIG. 2A) or TMT Mass Spec (FIG. 2B). Differentially expressed genes (DEGs) or proteins are shown in volcano plots (top) and GO Enrichment analysis was performed for the DEGs (bottom) (N=3). Plot (top) and GO Enrichment analysis was performed for the differentially expressed proteins (bottom) (N=3).

FIGS. 3A and 3B show heat maps showing the effects of immunostimulatory RNAs on IFN pathway-relevant gene levels. DEGs from RNA-seq (FIG. 3A) and differentially expressed proteins from TMT Mass Spec analyses (FIG. 3B) shown in FIG. 1B and FIG. 2 are presented here as heat maps (gene levels of the scrambled RNA control were set as 1; N=3).

FIGS. 4A and 4B show RNA-induced gene expression associated with type I interferon pathway. (FIG. 4A) Venn diagram showing differentially expressed ISGs from TMT Mass Spec by RNA-1 belong to type I or type II interferon stimulated genes. (FIG. 4B) Heat map of qPCR results showing RNA-I preferentially activates type I interferon pathway. A549 cells were transfected with RNA-1 or scrambled dsRNA control, collected at 48 hr and analyzed by qPCR (expression levels were normalized to GAPDH; gene levels induced by the RNA control were set as 1; N=3).

FIG. 5 shows the levels of IFN-β protein induced by RNA-1. A549 cells were transfected with RNA-1 (34 nM) for 48 h, and then supernatants were collected for detection of IFN-3 using ELISA. Scrambled RNA control NC-1 is used as negative (N=3).

FIG. 6 shows comparison of the immunostimulatory activities of different RNAs. A549-Dual cells were transfected with indicated duplex RNAs for 48 h, and then activation of the IFN pathway was measured by quantifying luciferase reporter activity. Data are shown as fold change relative to the scrambled RNA control (N=6).

FIG. 7A-7F show immunostimulatory RNAs induce IFN-I production through RIG-I-IRF3 pathway. (FIG. 7A) Wild-type (WT) HAP1 cells, IRF3 knockout HAP1 cells, or IRF7 knockout HAP1 cells were transfected with RNA-1 or scrambled RNA control for 48 h, and IFN-β mRNA levels were quantified by qPCR. Data are shown as fold change relative to the scrambled RNA control (N=3). Note that IRF3 knockdown completely abolished the IFN-β response. (FIG. 7B) IRF3 mRNA levels measured in A549 cells transfected with immunostimulatory RNA-4 or a scrambled RNA control, as determined by qPCR and 48 h post-transfection (data are shown as fold change relative to the control RNA; N=3). (FIG. 7C) Total IRF3 protein and phosphorylated IRF3 detected in A549 cells transfected with RNA-4 or scrambled RNA control at 48 h post transfection as detected by Western blot analysis (GAPDH was used as a loading control). (FIG. 7D) Immunofluorescence micrographs showing the distribution of phosphorylated IRF3 in A549 cells transfected with RNA-4 or scrambled RNA control at 48 h post transfection (Green, phosphorylated IRF3; blue, DAPI-stained nuclei; arrowheads, nuclei expressing phosphorylated IRF3). (FIG. 7E) Wild-type (WT) A549-Dual cells, RIG-I knockout A549-Dual cells, MDA5 knockout A549-Dual cells, or TLR3 knockout A549 cells were transfected with immunostimulatory RNA-4 or a scrambles RNA control and 48 h later, IFN-3 expression levels were quantified using the Quanti-Luc assay or qPCR (data are shown as fold change relative to the scrambled RNA control; N=6). Note that RIG-I knockout abolished the ability of the immunostimulatory RNAs to induce IFN-3. (FIG. 7F) SPR characterization of the binding affinity between cellular RNA sensors (RIG-I, MDA5, and TLR3) and RNA-1, which were immobilized on a streptavidin (SA) sensor chip. Equilibrium dissociation constant (KD), association rate constant (Ka), and dissociation rate constant (Kd) are labeled on the graphs.

FIG. 8 shows IRF3 knockout abolished the ability of immunostimulatory RNAs to induce IFN-I pathway associated genes. Wild-type (WT) HAP1 cells, IRF3 knockout HAP1 cells, or IRF7 knockout HAP1 cells were transfected with RNA-1 or a scrambled RNA control and STAT1, IL4L1, TRAIL, and IF16 mRNA levels were quantified by qPCR at 48 h post transfection. Data are presented as fold change relative to RNA control (N=3).

FIG. 9 shows RIG-I knockout abolished the induction effects of the immunostimulatory RNAs on IFN-β. Wild-type (WT) A549-Dual cells, RIG-I knockout A549-Dual cells, MDA5 knockout A549-Dual cells, or TLR3 knockout A549 cells were transfected with RNA-1, RNA-2, or a scramble RNA control and IFN-β mRNA levels were detected by Quanti-Luc assay in WT, RIG-I KO, and MDA5 KO A549-Dual cells or qPCR in TLR3 KO A549 cells at 48 h post transfection. Data are shown as fold change relative to the scrambled RNA control (N=6).

FIGS. 10A and 10B show TLR7/8 knockout or overexpression did not have effect on the immunostimulatory activity of RNA-1. (FIG. 10A) Graph showing that the overexpression of TLR7 in HEK cells had no effect on production of IFN-β induced by RNA-1. (FIG. 10B) Graph showing that the knockout of TLR8 in THP1 cells had no effect on IFN production induced by RNA 1. These cell lines are commercial and could be purchased from InvivoGen.

FIGS. 11A and 11B show the common motif mediates the formation of duplex RNA dimers via intramolecular G-quadruplex formed by GG overhang. (FIG. 11A) The image of native gel electrophoresis showing the formation of RNA-1 dimer. 1 μL of 10 uM RNA samples were loaded. RNA-9 and RNA-39 were used as negative and positive control, respectively. (FIG. 11B) The diagram shows the structure of ‘head-to-head’ RNA-1 dimer due to terminal G-G Hoogsteen paring.

FIG. 12A-12C show ‘CCC’ sequence is widely distributed in genome of human cells. (FIG. 12A) Graph showing the distribution of CCC in human mRNAs. (FIG. 12B) Graph showing the distribution of CCC in human lncRNAs. (FIG. 12C) Analysis of CCC distribution in human mRNAs and lncRNAs.

FIG. 13A-13D show immunostimulatory RNAs induce IFN-β production in differentiated human lung epithelial and endothelial cells in Organ Chips and exhibit broad spectrum inhibition of infection by H3N2 influenza virus, SARS-CoV-2, SARS-CoV-1, MERS-CoV, and HCoV-NL63. (FIG. 13A) Schematic diagram of a cross-section through the human Lung-on-Chip, which faithfully recapitulate human lung physiology and pathophysiology. (FIG. 13B) Human Lung Airway and Alveolus Chips were transfected with RNA-1 or scrambled RNA control by perfusion through both channels of the chip and 48 h later, the epithelial and endothelial cells were collected for detection of IFN-β mRNA by qPCR (data are presented as fold change relative to the RNA control; N=3; *, p<0.05; ***, p<0.001). (FIG. 13C) Effects of treatment with RNA-1 or a scrambled control in the human Lung Airway Chips or human Lung Alveolus Chips infected with influenza A/HK/8/68 (H3N2) (MOI=0.1) at 24 h after RNA-1 treatment. Viral load was determined by quantifying the viral NP gene by qPCR in cell lysates at 48 h after infection. Results are shown as fold change relative to RNA control; N=3; *, p<0.05. (FIG. 13D) Treatment with immunostimulatory duplex RNAs resulted in potent inhibition of multiple potential pandemic viruses, including SARS-CoV-2. Indicated cells were treated with RNA-1, RNA-2, or a scrambled control and infected with influenza A/HK/8/68 (H3N2) (MOI=0.1), SARS-CoV-2 (MOI=0.05), SARS-CoV-1 (MOI=0.01), MERS-CoV (MOI=0.01), and HCoV-NL63 (MOI=0.002), respectively, at 24 h after RNA transfection. Viral load was determined by quantifying the viral NP gene for H3N2, and the N gene for SARS-CoV-2 and HCoV-NL63 by qPCR in cell lysates at 48 h after infection; the viral load of SARS-CoV and MERS-CoV were determined by plaque assay at 48 h after infection. All results are shown as fold change relative to RNA control; N=3; *, p<0.05; ***, p<0.001.

FIG. 14 shows immunostimulatory RNA-mediated production of IFN in ACE2-overexpressing A549 cells. IFN-β and ISG15 levels were detected in cells transfected with RNA-1, RNA-2, or scramble dsRNA control by qPCR at 48 h post-transfection. The IFN-β or ISG15 level induced by the scramble dsRNA control was set as 1. Data are shown as fold change relative to the control (N=3).

FIG. 15A-15C show inhibition of native SARS-CoV-2 infection in vivo. (FIG. 15A) Reduction of viral load in the lungs of hamsters treated prophylactically with RNA-1 (20 μg in PBS) administered intranasally 1 day prior to intranasal administration of SARS-CoV-2 virus (10² PFU), on the day of infection, and 1 day post-infection, as measured one day later by qPCR for subgenomic RNA encoding SARS-CoV-2 N protein (sgRNA) (left; *, p0.030) or by quantifying viral titers in a plaque assay (right; *, p=0.032). (FIG. 15B) Reduction of viral load in the lungs of hamsters produced by administering RNA-1 (20 μg) intranasally once a day for two days beginning 1 day after intranasal administration of SARS-CoV-2 virus (10³ PFU) and measured one day later by qPCR for subgenomic RNA encoding SARS-CoV-2 N protein (* p=0.01). (FIG. 15C). Low (left) and high magnification (right) histological H&E-stained images of lungs from FIG. 15B that were treated with the delivery vehicle alone (top) or with vehicle containing RNA-1 (bottom) beginning 1 after infection (left bar, 2.5 mm; right bar, 100 μm).

FIG. 16 shows exemplary RNA duplex sequences. FIG. 16 presents SEQ ID NOs 5-30.

FIG. 17 shows a summary of characteristics of reported immunostimulatory RNAs.

FIGS. 18A-18C show immunostimulatory RNAs elicit responses with a stronger antiviral component and a lower proinflammatory component. (FIGS. 18A and 18B) Volcano plots showing significant upregulated genes or downregulated genes in RNA-1 transfected (FIG. 18A) or poly(I.C) transfected (FIG. 18B) A549 cells. Threshold for fold change=2, threshold for Padj=0.01. (FIG. 18C) Comparison of the immunostimulatory activities of different RNAs. A549-Dual cells were transfected with indicated duplex RNAs at a 5-fold serial dilution from 50 nM to 16 pM for 24 h, and then activation of the IFN pathway and NF-κB pathway was measured by quantifying luciferase reporter activity or alkaline phosphatase activity, respectively. N=4.

FIG. 19 shows viral titers at day 3 after challenge with SARS-CoV-2 virus in the lungs of K18-hACE2 mice treated with indicated RNAs or Vehicle (n=4). Data are plotted for individual mice and overlaid with mean±s.d.; **, p<0.01; ***, p<0.001; ****, p<0.0001.

FIG. 20 shows levels of IFN-β protein induced by RNA-1 and poly(I:C). A549 cells were transfected with RNA-1 or poly(I:C) (34 nM) for 48 h, and then supernatants were collected for detection of IFN-β using ELISA. Scrambled RNA control NC-1 is used as negative (N=3). *, P<0.05; n.s., not significant.

FIG. 21 shows RIG-I knockout completely abolished the immunostimulatory activity of RNAs. RIG-I knockout A549-Dual cells were transfected with poly (I:C), immunostimulatory RNAs or a scrambled RNA control (34 nM) and 48 h later, activation of the IFN pathway was measured by quantifying luciferase reporter activity. Data are shown as fold change relative to the scrambled RNA control (N=6). Note that poly (I:C) induced potent production of IFN in RIG-I knockout A549-Dual cells, while the immunostimulatory RNAs did not induce IFN in RIG-I knockout A549-Dual cells.

FIGS. 22A-22C show RNA-seq analysis to characterize host responses induced by RNA-1 and poly(I:C). (FIG. 22A) Principal component analysis of A549 cells transcriptomes when transfected with scrambled dsRNA (ctrl), RNA-1 (isRNA) or poly(I:C) for 48 hours. N=3. (FIG. 22B) Table showing induction of Type I and III IFN genes based on RNAseq data shown herein. (FIG. 22C) Heat map showing top upregulated inflammatory genes and top downregulated genes involved in ion transport and cell-cell adhesion in the poly(I:C) transfected but not in the isRNA (RNA-1) transfected A549 cells.

### DETAILED DESCRIPTION

The compositions and methods described herein relate, in part, to the discovery of immunomodulatory/immunostimulatory complex or RNA duplexes that induce interferon (IFN) production. The immunostimulatory complex or RNA duplexes described herein have the ability to induce robust innate immune responses and inhibit or treat diseases treatable with, or that benefit from increases in, interferons, including but not limited to viral, bacterial, fungal and/or parasitic infections, cancer and autoimmune diseases.

The following describes considerations to permit one of ordinary skill in the art to make and use the subject technology.

Interferons (IFN or IFNs) are a class of pleiotropic cytokines that are produced and released by immune cells as a part of the innate immune response to infections. IFNs have been used as a therapeutic in the treatment of autoimmune diseases (e.g., multiple sclerosis and lupus), many types of cancer, and viral infections. See, e.g., Paolicelli, D., Direnzo, V., & Trojano, M. (2009), Review of interferon beta-1b in the treatment of early and relapsing multiple sclerosis. Biologics: targets & therapy, 3, 369-376; Tamura T, Yanai H, Savitsky D, Taniguchi T., The IRF family transcription factors in immunity and oncogenesis. Annu Rev Immunol. (2008); McNab F, Mayer-Barber K, Sher A, Wack A, O'Garra A., Type I interferons in infectious disease Nat Rev Immunol. (2015), each of which is incorporated herein by reference in its entirety.

Immunomodulatory effects of IFNs are exerted on a wide range of cell types expressing receptors for the interferon polypeptide(s). Downstream effects of interferons allow for the regulation of the immune system by activating signal transducer and activator of transcription (STAT) complexes and other signaling molecules. STATs are a family of transcription factors that regulate the expression of a number of immune system genes. Interferon signaling pathways are known in the art—see e.g., Muller U, et al. Functional role of type I and type II interferons in antiviral defense. Science (1994); Honda et al, Immunity, 25, 349-360 (2006); Marchetti M, et al. Stat-mediated signaling induced by type I and type II interferons (IFNs) is differentially controlled through lipid microdomain association and clathrin-dependent endocytosis of IFN receptors. Mol Biol Cell (2006); Lee and Ashkar, Front. Immunol., 2018; Platanias LC. Mechanisms of type-I- and type-II-interferon-mediated signalling. Nat Rev Immunol. (2005) 5:375-86; each of which are incorporated herein by reference in their entirety.

The induction of interferon (IFN) production plays a critical role in human immune responses by ‘interfering’ with viral replication. Induction of IFN gene expression can lead to increased cellular resistance to infection, including but not limited to viral infection, by activating immune cells, (e.g., natural killer cells and macrophages), and increasing host defenses by upregulating antigen presentation by virtue of increasing the expression of major histocompatibility complex (MHC) antigens. There are a number of types of IFN genes and proteins, which are typically divided among three classes in humans: Type I IFN (IFN-α, IFN-β, IFN-ε, IFN-κ and IFN-ω), Type II IFN (IFN-γ), and Type III IFN. IFNs belonging to all three classes participate in fighting infection and regulating the immune system.

The regulation of IFN expression is complex and tightly controlled by interferon regulatory factors (IRFs). IRFs are a family of transcription factors that are involved in many aspects of the immune response, including development and differentiation of immune cells and regulating responses to pathogens. The functional role and signaling pathways of IRFs are known in the art, see e.g., Jefferries, Front. Immunol., 2019; and Bustamante et al. Clinical immunology, 5^{th} ed. (2019), which are incorporated herein by reference in their entirety. One such IRF, IRF3, is a positive regulator of type I interferon gene induction. IRF3 is an intracellular polypeptide that is activated downstream of the pattern recognition receptor, RIG-I, an intracellular RNA sensor. In particular, IRF3 can directly induce the expression of cytokines, such as IFN-β and in addition to type I IFNs, CXCL10, RANTES, ISG56, IL-12p35, IL-23, and IL-15, whilst inhibiting IL-12β and TGF-β.

The interferon pathways are involved in many diseases, including pathogenic infections caused by viruses, bacteria, fungi and parasites, as well as cancers, and autoimmune diseases. In many instances, an increase in interferon production is part of the natural response to infection, such that treatments that further promote such production can assist in fighting the infection. In other instances, notably some viral infections, including infection with the SARS-CoV-2 coronavirus, among others, the body's interferon response is not activated or is suppressed relative to that seen with other viruses or pathogens, such that a treatment that promotes interferon production can assist in fighting the infection. Therefore, the immunostimulatory complexes or RNA duplexes described herein can be used to prevent, mitigate, and/or treat diseases that benefit from or are treatable with agents that include interferons or that promote interferon production.

### Immunomodulatory Complex Compositions

The immunomodulatory complexes disclosed herein encompass a concatamer of oligonucleotide duplexes. In one aspect, the oligonucleotide duplex comprises an oligonucleotide strand having the structure 5′-C—N₁₆-GGG-3′ and an oligonucleotide strand having the structure 5′-C—N′₁₆-GGG-3′, wherein: N and N′ are each any of G, A, U and C; N16 is complementary to N′16; and duplexes in the concatamer are joined by Hoogsteen base pairing between 3′-GG overhanging dinucleotides on each duplex. N₁₆ is a minimum. However, N (and the corresponding N′ complementary sequence) can be longer. As discussed elsewhere herein, it is contemplated that the duplex can tolerate some degree of mismatch, but generally, no more than 5 of the N₁₆:N′₁₆ nucleobases should be mismatched. General rules for mismatches, if present, are also discussed elsewhere herein.

In another aspect herein, the immunostimulatory complex comprisES at least first and second RNA duplexes, each duplex comprising: a first strand comprising, from the 5′ terminus, the sequence 5′-C—N₁₉-3′, and a second strand comprising the sequence 5′-N′₁₉-GGG-3′, wherein: N and N′ are any of C, A, G, and U; N and N′ are complementary; the 3′ terminal GG dinucleotide of the second strand forms a 3′ GG dinucleotide overhang; the first duplex is complexed with the at least second duplex via Hoogsteen base pairing between the 3′ GG overhang on each duplex; and the first strand, at the 5′ terminus, does not comprise the sequence 5′-CUGA-3′. N₁₉ is a minimum. However, N (and the corresponding N′ complementary sequence) can be longer. As discussed elsewhere herein, it is contemplated that the duplex can tolerate some degree of mismatch, but generally, no more than 5 of the N₁₉:N′₁₉ nucleobases should be mismatched. General rules for mismatches, if present, are also discussed elsewhere herein.

In one embodiment, the immunostimulatory complex having SEQ ID NOs: 3 and 4, comprises one or more DNA nucleotides at the duplex end opposite the 5′-C. For example, the immunostimulatory complex comprises at least 4 or less, 3 or less, 2 or less, 1 or less DNA nucleotides at the duplex end opposite the 5′-C. In one embodiment, the immunostimulatory complex having SEQ ID NOs: 3 and 4, comprises a blunt end, a 5′ overhang or a 3′ overhang on the end opposite the 5′-C.

In one embodiment, the immunostimulatory complex and/or an oligonucleotide duplex as described herein is produced in a cell, e.g., by transcription of a template, e.g., a template introduced to the cell. In one embodiment, the immunostimulatory complex and/or oligonucleotide duplex produced in a cell is isolated.

In one embodiment, the immunostimulatory complex and/or the oligonucleotide duplex is synthetic.

As used herein, a “concatamer” encompasses a continuous DNA molecule that contains a plurality of copies of the same DNA sequence linked in series. Depending upon sequence, e.g., at the termini of the duplexes, monomer duplexes in a concatamer as described herein can be joined head-to-tail, head-to-head, or tail to tail. In one embodiment, the concatmer is a dimer of oligonucleotide monomer duplexes. In one embodiment, the concatmer comprises at least 2, at least 3, at least 4, at least 5, at least 6, at least 7, at least 8, at least 9, or at least 10 or more oligonucleotide duplexes.

In one embodiment, the RNA duplex or a complex comprising RNA duplexes has a 5′ monophosphate on the 5′ end of the first and/or second strand. In one embodiment, an RNA duplex or a complex comprising RNA duplexes has a 5′ diphosphate on the 5′ end of the first and/or second strand. In one embodiment, an RNA duplex or a complex comprising RNA duplexes has a 5′ triphosphate on the 5′ end of the first and/or second strand.

In one embodiment, the RNA duplex or a complex comprising RNA duplexes has a 5′ hydroxyl group on the 5′ end of the first and/or second strand.

In one embodiment, the oligonucleotide duplexes comprise double stranded RNA.

In some embodiments, the immunostimulatory RNA duplex has a length of 20-300, 20-250, 20-200, 20-150, 20-100, 20-50, 50-300, 50-250, 50-200, 50-150 or 50-100 nucleotides. In some embodiments, the immunostimulatory RNA duplex has a length of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49. 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides. These lengths are exclusive of any non-duplexed overhang (e.g., a 5′ or 3′ overhang) on either the first or second strand.

In one embodiment, the first and the second strand are complementary.

In one embodiment, SEQ ID NO: 1 and SEQ ID NO: 2 are complementary.

In one embodiment, SEQ ID NO: 1 and SEQ ID NO: 2 base pair via complementary sequences to form two duplexes that bind to each other via Hoogsteen base pairing. A Hoogsteen base pair is a variation of base-pairing in nucleic acids. In one format, a Hoogsteen base pair involves the N7 position of a purine base (as a hydrogen bond acceptor) and C6 amino group (as a donor), which bind the Watson-Crick (N3-C4) face of a pyrimidine base. However, another form of Hoogsteen base pairing pairs a terminal GG dinucleotide on one duplex with a terminal GG dinucleotide on another duplex via hydrogen bonding to form a so-called “G quadruplex” paired structure that joins the two duplexes end-to-end.

In one embodiment, SEQ ID NO: 1 and SEQ ID NO: 2 base pair to each other (via Watson-Crick base pairing) to form a duplex, and the duplexes dimerize via a G-quadruplex. In one embodiment, SEQ ID NO: 3 and SEQ ID NO: 4 base pair to each other (via Watson-Crick base pairing) to form a duplex, and the diplexes dimerize to form a G-quadrupleex. A G-quadruplex is a secondary structure formed in both DNA and RNA in guanine rich sequences. These secondary structures are helical and contain guanine tetrads that can form 1, 2, or 4 strands. A skilled person can determine if a G-quadruplex is formed via biochemical and biophysical assays known in the art. For example, a G-quadruplex can be identified in a DNA polymerase stop assay, or the topology of a G-quadruplex can be assessed by monitoring the positive or negative circular dichroism (CD) signals at specific wavelengths.

In one embodiment, the first and/or second strand of a duplex monomer comprises a two nucleotide overhang at its 3′ end. For example, the first and/or second strand can comprise a GG overhang at its 3′ end. Alternatively, the overhang can comprise DNA base pairs. For example, the first and/or second strand can comprise a TT overhang at its 3′ end. It is noted that a TT overhang does not have the capacity to form concatamers in the manner of a GG overhang.

In some embodiments, the immunostimulatory complexes or RNA duplexes described herein can be conjugated to an antigen or a biomolecule. In some embodiments, the immunostimulatory complex or RNA duplex described herein further comprises a linker. Such a linker can be used, for example, for conjugation of the RNA duplex to polynucleotide sequence encoding an antigen.

One aspect described herein provides a synthetic RNA duplex having a) a first strand having from 5′ to 3′ a GNNN sequence flanked by at least 22 nucleotides on each side; and b) a second strand having from 5′-3′ a GGGC sequence flanked by at least 22 nucleotides on each side; wherein the first and second strands are complementary to each other.

In one embodiment, SEQ ID NO: 5 and 6 are complementary to each other.

In one embodiment, the first strand has a two-nucleotide overhang at its 3′ end. In one embodiment, the second strand has a two-nucleotide overhang at its 3′ end. In one embodiment, the first and second strand each have a two-nucleotide overhang at their 3′ ends.

In one embodiment, the first strand has a two DNA nucleoside overhang at its 3′ end. In one embodiment, the second strand has a two DNA nucleoside overhang at its 3′ end. In one embodiment, the first and second strand each have a two DNA nucleoside overhang at their 3′ ends. Exemplary DNA nucleosides include thymidine, deoxyuridine, deoxyadenosine, deoxyguanosine, and deoxycytidine.

In one embodiment, the DNA nucleoside can be, for example, a thymidine, and thus forms a TT overhang at the 3′ end of a duplex.

In one embodiment, an RNA duplex as described herein has a 5′ monophosphate on the 5′ end of the first and/or second strand. In one embodiment, an RNA duplex as described herein has a 5′ diphosphate on the 5′ end of the first and/or second strand. In one embodiment, an RNA duplex as described herein has a 5′ triphosphate on the 5′ end of the first and/or second strand.

In one embodiment, an RNA duplex as described herein has a 5′ hydroxyl group on the 5′ end of the first and/or second strand.

Another aspect described herein provides a synthetic RNA duplex having a first and second strand having a sequence selected from SEQ ID NO: 7-32.

### Modifications Substitutions

It is contemplated that immunostimulatory complexes or RNA duplexes as described herein can comprise modified nucleotides including modifications to nucleobase and/or sugar-phosphate backbone moieties, as long as the modified nucleotides permit base pairing to the appropriate nucleotide on the opposing strand and as long as such modification(s) permit the resulting complex or duplex molecule to promote interferon production, e.g., as measured using methods known in the art or described herein. Such modifications can alter stability of the complex or duplex, e.g., by reducing susceptibility to enzymatic or chemical degradation, or can modify (increase or decrease) intra- or inter-molecular interactions, including but not limited to base-pairing interactions. RNA oligonucleotide duplex nucleobases include the purine bases adenine (A) and guanine (G), and the pyrimidine bases cytosine (C), and uracil (U) or modified or related forms thereof.

In one embodiment, the immunostimulatory complex or duplex comprises one or more modified ribonucleotides. Modified nucleotides can be located anywhere in the complex or duplex. In one embodiment, one or more modified nucleotides is, e.g., in the N₁₆ or N₁₆′, N₂₂ or N′₂₂ sequences of a complex or duplex as described herein, or they can be located elsewhere in the complex or duplex when the complex or duplex is longer than 48 nucleotides. It is contemplated that modifications that permit, for example, translation of an RNA comprising such modifications would be likely to be tolerated and retain immunostimulatory/interferon-inducing activity in the context of the complexes or duplexes described herein.

It is contemplated that one or more, two or more, three or more, including all four of the ribonucleotides 5′-C—N₁₆-GGG-3′ can be modified in a given duplex molecule. It is further contemplated that one or more, two or more, three or more, four or more, five or more, including all six of the ribonucleotides 5′-C—N′₁₆-GGG-3′ can be modified in a given duplex molecule. It is further contemplated that the N₁₆ or N′₁₆ sequence, where comprised by a complex or duplex as described herein, can include modifications to one or more, two or more, three or more, four or more, five or more, six or more, seven or more, eight or more, nine or more, ten or more, eleven or more, twelve or mote, thirteen or more, fourteen or more, fifteen or more, up to and including all ribonucleotides comprising one or more nucleobase or ribose-phosphate backbone modifications. Similarly, when the N-N′ duplex comprises more than 16 ribonucleotides, any one or any combination of them, up to and including all of them, can include one or more modifications to the nucleobase or ribose-phosphate backbone structure.

Exemplary nucleic acid modifications include, but are not limited to, nucleobase modifications, sugar modifications, inter-sugar linkage modifications, conjugates (e.g., ligands), and combinations thereof. In one embodiment, a modification does not include replacement of a ribose sugar with a deoxyribose sugar as occurs in deoxyribonucleic acid. Nucleic acid modifications are known in the art, see, e.g., US20160367702A1; US20190060458A11; U.S. Pat. Nos. 8,710,200; and 7,423,142, which are incorporated herein by reference in their entireties.

Exemplary modified nucleobases include, but are not limited to, thymine (T), inosine, xanthine, hypoxanthine, nubularine, isoguanisine, tubercidine, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and 0-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613. Exemplary sugar modifications include, but are not limited to, 2′-Fluoro, 3′-Fluoro, 2′-OMe, 3′-OMe, and acyclic nucleotides, e.g., peptide nucleic acids (PNA), unlocked nucleic acids (UNA) or glycol nucleic acid (GNA).

In some embodiments, a nucleic acid modification can include replacement or modification of an inter-sugar linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH2-N(CH3)-N(CH3)-), amide-3 (3′-CH2-C(═O)—N(H)-5′) and amide-4 (3′-CH2-N(H)-C(═O)-5′), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S-CH2-O-5′), formacetal (3′-O-CH2-O-5′), oxime, methyleneimino, methykenecarbonylamino, methylenemethylimino (MMI, 3′-CH2-N(CH3)-O-5′), methylenehydrazo, methylenedimethylhydrazo, methyleneoxymethylimino, ethers (C3′-O-C5′), thioethers (C3′-S-C5′), thioacetamido (C3′-N(H)—C(═O)—CH2-S-C5′, C3′-O—P(O)—O—SS-C5′, C3′-CH2-NH—NH-C5′, 3′-NHP(O)(OCH3)-O-5′ and 3′-NHP(O)(OCH3)-O-5′

In some embodiments, nucleic acid modifications can include peptide nucleic acids (PNA), bridged nucleic acids (BNA), morpholinos, locked nucleic acids (LNA), glycol nucleic acids (GNA), threose nucleic acids (TNA), or other xeno nucleic acids (XNA) described in the art.

In another embodiment of any of the aspects, the complex or duplex described herein comprises a linker. For example, the linker can simply be a nucleic acid backbone linkage e.g., phosphodiester linkage. In addition, the nucleic acid linkers can all be the same, all different, or some are the same and some are different.

In some embodiments of any of the aspects, the linker or spacer can be selected from the group consisting of: photocleavable linkers, hydrolyzable linkers, redox cleavable linkers, phosphate-based cleavable linkers, acid cleavable linkers, ester-based cleavable linkers, peptide-based cleavable linkers, and any combinations thereof. In some embodiments, the cleavable linker can comprise a disulfide bond, a tetrazine-trans-cyclooctene group, a sulfhydryl group, a nitrobenzyl group, a nitoindoline group, a bromo hydroxycoumarin group, a bromo hydroxyquinoline group, a hydroxyphenacyl group, a dimethozybenzoin group, or a combination thereof.

In some embodiments, the immunostimulatory complex or RNA duplexes described herein are cross-linked such that the complementary strands are covalently joined. Such cross-linking can provide, for example, improved complex or duplex stability, such that the central sequences (SEQ ID NOs 1 and 2) identified herein is better retained in its active conformation. In some embodiments, the cross-linking moiety can be a chemical functional group. In some embodiments, said chemical functional group is selected from the group consisting of: azide, alkyne, tetrazine, DBCO, thiol, amine, carbonyl, carboxyl group, and any combinations thereof.

In some embodiments, the immunostimulatory complex or RNA duplexes described herein are cross-linked by a photo-cross linking moiety. Non-limiting examples of photo-crosslinking moieties include, 3-Cyanovinylcarbazole (CNVK) nucleotide; 5-bromo deoxycytosine; 5-iodo deoxycytosine; 5-bromo deoxyurdine; 5-iodo deoxyuridine; and nucleotides comprising an aryl azide (AB-dUMP), benzophenone (BP-dUMP), perfluorinated aryl azide (FAB-dUMP) or diazirine (DB-dUMP).

In some embodiments, the immunostimulatory complex or RNA duplexes described herein are conjugated to a pharmaceutically acceptable carrier. In other embodiments, the immunostimulatory complex or RNA duplexes described herein are admixed with a pharmaceutically acceptable carrier.

In some embodiments of any of the aspects, immunostimulatory complex or RNA duplexes described herein are conjugated to an antigen or antigenic fragment thereof or a sequence encoding an antigen or antigenic fragment thereof.

In some embodiments, an immunostimulatory complex or RNA duplex as described herein can be fused to or otherwise include a sequence encoding an antigen. Such a composition will include a single-stranded RNA sequence encoding the antigen, fused to or in complex with an immunostimulatory complex or duplex as described herein. RNA vaccines and RNAs encoding vaccine antigens are known in the art. RNA vaccines are further described in, e.g., International Patent Application Nos WO2009040443; WO2012138453A1; WO2012138453A1; and WO2013052523A1; the contents of which are incorporated herein by reference in their entireties.

Introduction of such a composition to a cell can result in both production of antigen to stimulate an adaptive immune response and concomitant stimulation of an interferon response. In one embodiment, the antigen sequence is fused to the 3′ end of the dsRNA. In one embodiment, the antigen is fused to the 3′ end of the dsRNA such that it does not interfere or alter the IFN-inducing effect, which involves the 5′ end of the dsRNA.

### Methods of Preparing Complexes and Duplexes

The immunostimulatory complex or RNA duplexes described herein can be prepared by synthetic methods known in the art including, but not limited to, chemical synthesis, including but not limited to a nucleoside phosphoramidite approach, or in vitro transcription among others. Methods for chemical synthesis to include modified nucleotides are also known in the art.

In in vitro transcription, polymerases can be used including, but not limited to, bacteriophage polymerase such as T7 polymerase, T3 polymerase and SP6 polymerase, viral polymerases, and E. coli RNA polymerase.

Oligonucleotide strands can be isolated from a sample using RNA extraction and purification methods know in the art. These methods include but are not limited to column purification, ethanol precipitation, phenol-chloroform extraction, or acid guanidinium thiocyanate-phenol chloroform extraction (AGPC). Following isolation of a single stranded oligonucleotide, hybridizing and/or annealing the top and bottom strands can be performed to form the duplex secondary structure.

As used herein, the term “hybridizing”, “hybridize”, “hybridization”, “annealing”, or “anneal” are used interchangeably in reference to the pairing of complementary nucleic acids using any process by which a strand of nucleic acid joins with a complementary strand through base pairing to form a hybridization complex. In other words, the term “hybridization” refers to the process in which two single-stranded polynucleotides bind non-covalently to form a double-stranded polynucleotide. The resulting double-stranded polynucleotide is a “hybrid” or “duplex.” Conditions for forming hybridized or duplexed sequences are known to those of skill in the art, and generally include salt concentration and temperature at or near normal physiological conditions, e.g., intracellular conditions. Generally, hybridization to form duplexes as described herein can be performed with each strand present in substantially equimolar concentrations.

Following synthesis, hybridization and, optionally, removal of non-duplexed strands, the immunostimulatory RNA duplexes can be characterized by any method known in the art, e.g., liquid chromatography, mass spectrometry, next generation sequencing, polymerase chain reaction (PCR), gel electrophoresis, or any other method of identifying nucleoside sequences, secondary structures, chemical composition, expression, thermodynamics, binding, or function.

For further characterization of the immunostimulatory complexes or duplexes described herein, 5′-monophosphate can be detected, for example, by a splinted ligation assay. See e.g., Shoenberg et al, Nat Chem Biol 3(9) (2007) and Celesnik H et al. Initiation of RNA decay in Escherichia coli by 5′ pyrophosphate removal. Mol Cell. 2007; 27:79-90, which are incorporated herein by reference in their entireties. By carefully optimizing reaction conditions and comparing ligated with unligated RNA this assay yields quantitative data regarding the amount of RNA with a 5′ monophosphate end. Alternative assays useful in characterizing the immunostimulatory complexes or duplexes described herein include Native RNA gel (i.e., as described in, e.g., Booy, E P, et al. Methods Mol Biol. 2012; 941:69-81, which is incorporated herein by reference in its entirety) and nuclear magnetic resonance (NMR), i.e., using standard techniques in the art.

In order to improve the stability or produce any of the oligonucleotide modifications described above, immunostimulatory complex or RNA duplex as described herein, may be chemically modified in a suitable manner. As noted above, modifications can be made in order to meet the requirements of stability of the complex or RNA duplexes toward extra- and intracellular enzymes and ability to penetrate through the cell membrane for human therapeutic applications. See, e.g., Uhlmann, E.; Peyman, A. Chem. Rev. 1990, 90, 544; Milligan, J. F.; Matteucci, M. D.; Martin, J. C. J. Med. Chem. 1993, 36, 1923; Crooke, S. T.; Lebleu, B., Eds. 1993, Antisense research and applications; CRC Press: Boca Raton, FL; and Thuong, N. T.; Helene, C. Angew. Chim. Int. Ed. 1993, 32, 666. Chemical modifications to nucleic acids may include introduction of heterocyclic bases, phosphate backbone modifications, sugar moiety modifications, and attachment of conjugated groups. See Beaucage, S. L.; Iyer, R. P. Tetrahedron 1993, 49, 1925; Beaucage, S. L.; Iyer, R. P. Tetrahedron 1993, 49, 6123; Manoharan, M. Antisense Technology, 2001, S. T. Crooke, ed. (Marcel Dekker, New York); and Manohran, M. Antisense & Nucleic acid Development 2002, 12, 103, Schweitzer, B. A.; Kool, E. T. J. Org. Chem. 1994, 59, 7238; Schweitzer, B. A.; Kool, E. T. J. Am. Chem. Soc. 1995, 117, 1863; Moran, S. Ren, R. X.-F. Rumney, S.; Kool, E. T. J. Am. Chem. Soc. 1997, 119, 2056; Guckian, K. M.; Kool, E. T. Angew. Chem. Int. Ed. Engl. 1997, 36, 2825; and Mattray, T. J.; Kool, E. T. J. Am. Chem. Soc. 1998, 120, 6191. For additional information see Fire, A.; Xu, S.; Montgomery, M. K.; Kostas, S. A.; Driver, S. E.; Mello, C. C. Nature, 1998, 391, 806; Elbashir, S. M.; Harborth, J.; Lendeckel, W.; Yalcin, A.; Weber, K.; Tuschl, T. Nature, 2001, 411, 494; McManus, M. T. Sharp, P. A. Nature Reviews Genetics, 2002, 3, 737; Hannon, G. J. Nature, 2002, 418, 244; and Roychowdhury, A.; Illangkoon, H.; Hendrickson, C. L.; Benner, S. A. Org. Lett. 2004, 6, 489, which are incorporated herein by reference in their entireties.

For some therapeutic purposes, immunostimulatory complex or RNA duplexes described herein should have a degree of stability in serum to permit distribution and cellular uptake. The prolonged maintenance of therapeutic levels of the oligonucleotides in serum will have a significant effect on the distribution and cellular uptake and unlike conjugate groups that target specific cellular receptors, the increased serum stability will affect all cells.

Chemical modifications can also include the addition of ligands, linkers, and antigens. For example, a ligand can improve stability, hybridization thermodynamics with a target nucleic acid, targeting to a particular tissue or cell-type, or cell permeability, e.g., by an endocytosis-dependent or independent mechanism. Oligonucleotides bearing peptide (e.g. antigen) conjugates can be prepared using procedures known in the art. See Trufert et al., Tetrahedron 1996, 52, 3005; and Manoharan, “Oligonucleotide Conjugates in Antisense Technology,” in Antisense Drug Technology, ed. S.T. Crooke, Marcel Dekker, Inc., 2001, each of which is hereby incorporated by reference.

### Pharmaceutical Compositions

The methods and immunostimulatory complex or duplex compositions described herein can further comprise formulating the immunostimulatory complex or RNA duplexes described herein with a pharmaceutically acceptable carrier.

In some embodiments of any of the aspects, the method further comprises formulating the immunostimulatory complex or duplex with a pharmaceutically acceptable carrier and an antigen or a nucleic acid sequence encoding an antigen. Such formulations exploit the immunostimulatory complex or RNA duplexes as described herein to provide an adjuvant effect, e.g., when the formulation is administered as or in conjunction with a vaccine. In some embodiments of any of the aspects, the method further comprises formulating the immunostimulatory complex or duplexes with a pharmaceutically acceptable carrier, an antigen or a nucleic acid sequence encoding an antigen, and a separate adjuvant.

For clinical use of the methods and compositions described herein, administration of the immunostimulatory complex or RNA duplex described herein can include formulation into pharmaceutical compositions or pharmaceutical formulations for parenteral administration, e.g., intravenous; mucosal, e.g., intranasal; ocular, or other mode of administration. In some embodiments, the immunostimulatory complex or RNA duplex described herein can be administered along with any pharmaceutically acceptable carrier compound, material, or composition which results in an effective treatment in the subject. Thus, a pharmaceutical formulation for use in the methods described herein can contain the immunostimulatory complex or RNA duplex described herein in combination with one or more pharmaceutically acceptable ingredients. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an immunostimulatory complex or RNA duplex as described herein. Each carrier must be “acceptable” in the sense of being compatible with the other ingredients of the formulation and not injurious to the patient. The terms “excipient,” “carrier,” “pharmaceutically acceptable carrier” or the like are used interchangeably herein.

The immunostimulatory complexes or RNA duplexes described herein can be formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) transdermally; (3) transmucosally; (4) via bronchoalveolar lavage.

In some embodiments, the compositions described herein comprise a particle or polymer-based vehicle. Exemplary particle or polymer-based vehicles include, but are not limited to, nanoparticles, microparticles, polymer microspheres, or polymer-drug conjugates.

In one embodiment of any of the aspects, the compositions described herein further comprise a lipid vehicle. Exemplary lipid vehicles include, but are not limited to, liposomes, phospholipids, micelles, lipid emulsions, and lipid-drug complexes.

Formulations can be adapted for delivery to the airway, e.g., to address respiratory infection. Such formulations can be adapted for delivery as an aerosol, e.g., for inhalation. In some embodiments, the compositions described herein are formulated for aerosol administration, nebulizer administration, or tracheal lavage administration. In some embodiments, the composition is formulated for intravenous, intramuscular, intraperitoneal, subcutaneous, or intrathecal administration.

For use as aerosols, the compositions described herein can be prepared in a solution or suspension and may be packaged in a pressurized aerosol container together with suitable propellants, for example, hydrocarbon propellants like propane, butane, or isobutane with conventional excipients.

The immunostimulatory complex or RNA duplex compositions described herein can also be administered in a non-pressurized form such as in a nebulizer or atomizer that reduces a liquid to a fine spray. Preferably, by such nebulization small liquid droplets of uniform size are produced from a larger body of liquid in a controlled manner. Nebulization can be achieved by any suitable means therefor, including by using many nebulizers known and marketed today. For example, an AEROMIST™ pneumatic nebulizer available from Inhalation Plastic, Inc. of Niles, Ill.

When the active ingredients are adapted to be administered, either together or individually, via nebulizer(s) they can be in the form of a nebulized aqueous suspension or solution, with or without a suitable pH or tonicity adjustment, either as a unit dose or multi-dose device.

Furthermore, any suitable gas can be used to apply pressure during the nebulization, with preferred gases to date being those which are chemically inert. Exemplary gases including, but not limited to nitrogen, argon, or helium can be used to advantage.

In some embodiments, the compositions described herein can also be administered directly to the airways in the form of a dry powder. Thus, the immunostimulatory complex or RNA duplexes can be administered via an inhaler. Exemplary inhalers include metered dose inhalers and dry powdered inhalers.

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Commonly used propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti-oxidant and/or a stabilizing agent.

A dry powder inhaler (i.e., Turbuhaler™ (Astra AB)) is a system operable with a source of pressurized air to produce dry powder particles of a pharmaceutical composition that is compacted into a very small volume.

Dry powder aerosols for inhalation therapy are generally produced with mean diameters primarily in the range of <5 μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations including the immunostimulatory complexes or RNA duplexes described herein. These can be intermixed with lactose, or other inert powders acceptable for intrabronchial administration. The powder compositions can be administered via an aerosol dispenser or encased in a breakable capsule which may be inserted by the patient or clinician into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are described, for example, by Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), the contents of each of which are incorporated herein by reference in their entirety.

In certain embodiments, the dosage range of the immunostimulatory complexes, RNA duplexes in pharmaceutical compositions for aerosol delivery is between 0.1 mg/ml to 1 mg/ml. In one embodiment, the dosage range is between 0.2 mg/ml to 1 mg/ml; 0.3 mg/ml to 1 mg/ml; 0.4 mg/ml to 1 mg/ml; 0.5 mg/ml to 1 mg/ml; 0.6 mg/ml to 1 mg/ml; 0.7 mg/ml to 1 mg/ml; 0.8 mg/ml to 1 mg/ml; 0.9 mg/ml to 1 mg/ml; 0.1 mg/ml to 1 mg/ml; 0.2 mg/ml to 0.8 mg/ml; 0.2 mg/ml to 0.6 mg/ml; 0.2 mg/ml to 0.4 mg/ml; 0.3 mg/ml to 0.8 mg/ml; 0.3 mg/ml to 0.6 mg/ml; 0.3 mg/ml to 0.5 mg/ml; 0.4 mg/ml to 0.8 mg/ml; 0.4 mg/ml to 0.6 mg/ml; 0.5 mg/ml to 0.9 mg/ml; or 0.6 mg/ml to 0.9 mg/ml.

In addition to chemical modification of the immunostimulatory complex or RNA duplexes described herein, efforts aimed at improving the transmembrane delivery of nucleic acids and oligonucleotides have utilized protein carriers, antibody carriers, liposomal delivery systems, electroporation, direct injection, cell fusion, viral vectors, and calcium phosphate-mediated transformation. U.S. Pat. Nos. 7,423,142 B2, 7,786,290 B2, 8,598,139 B2, 8,808,747 B2, 10,125,369 B2, 10,130,649 B2, and U.S. patent publication 2018/0369419 A1, each of which is incorporated herein by reference, describe formulations for delivery of mRNA, siRNA, and dsRNA compositions to skin, blood, liver, and other target tissues or organs. As but one example, U.S. Pat. No. 8,598,139 B2 provides several examples of nucleic acid-lipid particle formulations for delivery; see, e.g., columns 42-48. Where the interferon-inducing molecules disclosed herein also have complex or duplex characteristics, it is specifically contemplated that formulations for delivery of siRNA compositions to such tissues can be used to deliver the complex or duplexes disclosed herein.

In some embodiments the immunostimulatory complex or RNA duplexes as described herein are formulated in a composition comprising micelles, amphiphilic carriers, polymers, cyclodextrins, liposomes, and encapsulation devices.

Immunostimulatory complexes and RNA duplexes can be formulated in a nanoparticle.

Microemulsification technology can improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

The immunostimulatory complex or RNA duplexes as described herein can be formulated with an amphiphilic carrier. Amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc. (produced and distributed by a number of companies in USA and worldwide).

The immunostimulatory complex or RNA duplexes as described herein can be formulated with hydrophilic polymers. Hydrophilic polymers are water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Other hydrophilic polymers which may be suitable include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a pharmaceutical composition as described herein comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In certain embodiments, a pharmaceutical composition described herein is formulated as a liposome. Liposomes can be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993.

In some embodiments of any of the aspects, immunostimulatory complex or RNA duplexes as described herein can be conjugated to an antigen or antigenic fragment thereof and formulated as a vaccine composition. Therapeutic formulations of the immunostimulatory complex or RNA duplexes as described herein can be prepared for storage by mixing the immunostimulatory complex or RNA duplex having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Vaccine or other pharmaceutical compositions comprising an immunostimulatory complex or RNA duplex composition as described herein can contain a pharmaceutically acceptable salt, typically, e.g., sodium chloride, and preferably at about physiological concentrations. The formulations of vaccine or other pharmaceutical compositions described herein can contain a pharmaceutically acceptable preservative. In some embodiments, the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. The formulations of vaccine or other pharmaceutical compositions described herein can include a pharmaceutically acceptable surfactant at a concentration of 0.005 to 0.02%.

Therapeutic pharmaceutical compositions described herein can also contain more than one active compound as necessary for the particular indication being treated, preferably those with complementary activities that do not adversely affect each other.

In some embodiments in which the complex or duplexes are formulated for use in or with a vaccine, the vaccine composition can be formulated with the complex or duplex as an adjuvant. In other embodiments the vaccine composition can be formulated with the immunostimulatory complex or RNA duplex and an additional adjuvant, e.g., as known in the art.

As used herein in the context of immunization, immune response and vaccination, the term “adjuvant” refers to any substance than when used in combination with a specific antigen produces a more robust immune response than the antigen alone. When incorporated into a vaccine formulation, an adjuvant acts generally to accelerate, prolong, or enhance the quality of specific immune responses to the vaccine antigen(s).

Adjuvants typically promote the accumulation and/or activation of accessory cells or factors to enhance antigen-specific immune responses and thereby enhance the efficacy of vaccines, i.e., antigen-containing or encoding compositions used to induce protective immunity against the antigen.

Adjuvants, in general, include adjuvants that create a depot effect, immune-stimulating adjuvants, and adjuvants that create a depot effect and stimulate the immune system. An adjuvant that creates a depot effect is an adjuvant that causes the antigen to be slowly released in the body, thus prolonging the exposure of immune cells to the antigen. This class of adjuvants includes but is not limited to alum (e.g., aluminum hydroxide, aluminum phosphate); emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720; AirLiquide, Paris, France); MF-59 (a squalene-in-water emulsion stabilized with Span 85 and Tween 80; Chiron Corporation, Emeryville, Calif.); and PROVAX™ (an oil-in-water emulsion containing a stabilizing detergent and a micelle-forming agent; IDEC Pharmaceuticals Corporation, San Diego, Calif).

An immune-stimulating adjuvant is an adjuvant that causes activation of a cell of the immune system. It may, for instance, cause an immune cell to produce and secrete cytokines and interferons. This class of adjuvants includes but is not limited to saponins purified from the bark of the Q. saponaria tree, such as QS21 (a glycolipid that elutes in the 21st peak with HPLC fractionation; Aquila Biopharmaceuticals, Inc., Worcester, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). This class of adjuvants also includes CpG DNA.

Adjuvants that create a depot effect and stimulate the immune system are those compounds which have both of the above-identified functions. This class of adjuvants includes but is not limited to ISCOMS (immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxypropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

The active ingredients of the pharmaceutical compositions described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

In some embodiments, sustained-release preparations can be used. Suitable examples of sustained-release preparations include semipermeable matrices of solid hydrophobic polymers containing an antigen or fragment thereof described herein in which the matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated, the antigen or fragment thereof can remain in the body for a long time, denature, or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S— bond formation through thio-disulfide interchange, stabilization can be achieved by modifying sulfhydryl residues, lyophilizing from acidic solutions, controlling moisture content, using appropriate additives, and developing specific polymer matrix compositions.

### Immunostimulatory Activity

The immunostimulatory complex or RNA duplexes, pharmaceutical compositions, and vaccine compositions described herein can be administered to a subject in need of immunostimulation, and particularly a subject in need of or that would likely to benefit from induction of interferon production. In various embodiments, the interferon-inducing activity is therapeutic on its own, in combination with one or more anti-infectives (e.g., antiviral, antibacterial, antifungal or anti-parasitic), in combination with one or more anti-cancer agents, or in combination with one or more therapeutics for autoimmune disease.

Various aspects provided herein relate to a method for inducing IFN production. In various embodiments, the complex, the concatamers, or RNA duplexes described herein induce interferon (IFN) production in a cell.

In one embodiment, the IFN production is type I IFN, type II IFN, or type III IFN production.

In one embodiment, the IFN production is type I IFN production. In one embodiment, the type I IFN production is IFN-α, IFN-β, IFN-ε, IFN-κ or IFN-ω production.

One aspect is a method of inducing IFN production comprising administering to a subject in need thereof an immunostimulatory complex or duplex described herein or a pharmaceutical composition thereof, whereby IFN production is increased following administration.

Another aspect is a method of inducing IFN production comprising administering to a subject in need thereof an immunostimulatory complex or duplex, e.g., naturally occurring or synthetic, as described herein or a pharmaceutical composition thereof, whereby IFN production is increased following administration.

Immunostimulatory activity can be determined, for example, by detecting and measuring the levels of cytokine and interferon production in a biological sample (e.g., serum).

Methods for detecting, measuring, and determining the levels of IFN in a biological sample are known in the art. IFN polypeptide levels can be detected, for example, via immunoassay. ThermoFisher Scientific sells an ELISA-based kit for measuring human interferon gamma levels —see Catalog #29-8319-65. IFN gene expression can also be detected. Methods of measuring gene expression are known in the art, e.g., PCR, microarrays, and immunodetection methods, such as Western blotting and immunocytochemistry, among others. For example, Quantitative reverse transcription polymerase chain reaction (qPCR) analysis can be performed using kits and arrays commercially available from, e.g., Applied Biosystems™—see Applied Biosystems® TaqMan® Array Human Interferon Pathway, catalog #4414154. See also, de Veer M J et al. Functional classification of interferon-stimulated genes identified using microarrays. J Leukoc Biol. (2001) 69:912-20, which are incorporated herein by reference in their entireties.

Antibodies specific for a class of interferon polypeptides (e.g., IFN-γ) are known in the art and can be used in immunohistochemistry, immunofluorescence, and Western Blotting, e.g., commercially available from Abcam™.

Interferon levels and activity can also be determined using a reporter assay or a bioassay. For example, reporter assays for the detection of bioactive type I interferons are available from InvovGen® by monitoring the activation of the ISGF3 pathway. See, e.g., Rees et al. J Immunol Methods, (2018).

Viral infection assays can also be used to determine the effect of the immunostimulatory complex or RNA duplexes on viral protection. For example, IFN activity can be measured by the level of protection of a cell line against cell death after infection with a virus as compared with a relevant control. See, e.g., Barber et al. Host defense, viruses and apoptosis. Cell Death Differ 8, 113-126, doi: 10.1038/sj.cdd.4400823 (2001); and Liu, S. et al. Science 347, (2015), and which are incorporated herein by reference in its entirety.

In addition, relevant animal models and human in vitro engineered platforms can also be used to detect interferon production directly or indirectly. Any model known in the art can be used. See, e.g., Si, L. et al. Human organs-on-chips as tools for repurposing approved drugs as potential influenza and COVID19 therapeutics in viral pandemics. bioRxiv, doi:10.1101/2020.04.13.039917 (2020); Van den Broek M F, Muller U, Huang S, Zinkernagel R M, Aguet M. Immune defence in mice lacking type I and/or type II interferon receptors. Immunol Rev. (1995).

Providing protection against the relevant pathogen includes stimulating the immune system such that later exposure to a microorganism, antigen, or antigen fragment thereof (e.g., an antigen on or in a live pathogen) triggers a more effective immune response than if the subject was naïve to the antigen. Protection can include faster clearance of the pathogen, reduced severity and/or time of symptoms, and/or lack of development of disease or symptoms. As compared with an equivalent untreated control, such reduction is by at least 5%, 10%, 20%, 40%, 50%, 60%, 80%, 90%, 95%, 99% or more as measured by any standard technique.

In various embodiments, the immunostimulatory complexes and RNA duplexes described herein activate the RIG-I-IRF3 pathway. In one embodiment, a concatamer described herein activates the RIG-I-IRF3 pathway. Activation of the RIG-I-IRF3 pathway can be assessed by a skilled person, e.g., via determining whether downstream targets of IRF3 are induced; e.g., the expression of cytokines, such as IFN-β, type I IFNs, CXCL10, RANTES, ISG56, IL-12p35, IL-23, and IL-15. Further, one can determine if IL-12β and/or TGF-β expression has been inhibited. A skilled person can assess mRNA or protein expression levels via PCR-based assays or western-blotting, respectively.

### Methods of Treatment

The immunostimulatory complex or RNA duplexes described herein can be used for treating IFN-associated diseases, including infection by a wide range of viral, bacterial, fungal, and parasitic pathogens, as well as cancer, and autoimmune diseases, in addition to inhibiting influenza virus infection.

A disease or medical condition is considered to be associated with interferons if administration or induction of interferon production treats the disease or condition. Some diseases or disorders involve interferon induction as part of the healing or recovery process, while in others, the pathology is characterized by deficient, low or nonexistent production of interferons, e.g., IFN, Type I IFN, IFN-α, IFN-β, IFN-ε, IFN-κ and IFN-ω, Type II IFN (IFN-γ), and Type III IFN.

Described herein is a method of treating an infection in a subject in need thereof, the method comprising administering to the subject an immunostimulatory complex or RNA duplex described herein.

In one embodiment of this or any of the aspects, the complex or RNA duplex is sufficient to induce interferon (IFN) production in a cell contacted with the duplex. In another embodiment, administering the complex or RNA duplex to a subject in need thereof is sufficient to increase the levels or activity of IFN. In another embodiment, administering the complex or RNA duplex to a subject in need thereof is sufficient to increase an immune response in the subject. In another embodiment, the immune response is an anti-viral response.

Without limitations, the immunostimulatory complex or RNA duplexes described herein can be used to treat a microbial infection. Non-limiting examples of microbes that can cause a microbial infection include viruses, bacteria, fungi and parasites.

In another embodiment, the microbial infection is chronic. In one embodiment, the microbial infection is acute. An acute infection is a short term infection, persisting less than 2 weeks, while a chronic infection is long term, and persists longer than two weeks. The method for treating an acute infection can be the same method used to treat a chronic infection. In contrast, a different method can be used to treat an acute and chronic infection.

In some embodiments of any of the aspects, the microbial infection is a systemic infection. As described herein, “systemic infection” refers to an infection that has spread throughout the body, for example, an infection that is present in the blood. Non-limiting examples of systemic infections include bacterial sepsis and endotoxin shock.

In some embodiments, the microbial infection is caused by a bacterium. Non-limiting examples of bacterial infections that can be treated or prevented by administering an immunostimulatory complex or RNA duplex described herein includes but is not limited to Aeromonas infection, African tick bite fever, American tick bite fever (Rickettsia parkeri infection), Arcanobacterium haemolyticum infection, Bacillary angiomatosis, Bejel (endemic syphilis), Blastomycosis-like pyoderma (pyoderma vegetans), Blistering distal dactylitis, Botryomycosis, Briii-Zinsser disease, Brucellosis (Bang's disease, Malta fever, undulant fever), Bubonic plague, Bullous impetigo, Cat scratch disease (cat scratch fever, English-Wear infection, inoculation lymphoreticulosis, subacute regional lymphadenitis), Cellulitis, Chancre, Chancroid (soft chancre, ulcus molle), Chlamydia infection, Chronic lymphangitis, Chronic recurrent erysipelas, Chronic undermining burrowing ulcers (Meleney gangrene), Chromobacteriosis infection, Condylomata lata, Cutaneous actinomycosis, Cutaneous anthrax infection, Cutaneous C. diphtheriae infection (Barcoo rot, diphtheric desert sore, septic sore, Veldt sore), Cutaneous group B streptococcal infection, Cutaneous Pasteurella hemo/ytica infection, Cutaneous Streptococcus iniae infection, Dermatitis gangrenosa (gangrene of the skin), Ecthyma, Ecthyma gangrenosum, Ehrlichiosis ewingii infection, Elephantiasis nostras, Endemic typhus (murine typhus), Epidemic typhus (epidemic louse-borne typhus), Erysipelas (ignis sacer, Saint Anthony's fire), Erysipeloid of Rosenbach, Erythema marginatum, Erythrasma, External otitis (otitis externa, swimmer's ear), Felon, Flea-borne spotted fever, Flinders Island spotted fever, Flying squirrel typhus, Folliculitis, Fournier gangrene (Fournier gangrene of the penis or scrotum), Furunculosis (boil), Gas gangrene (Clostridial myonecrosis, myonecrosis), Glanders (Equinia, farcy, malleus), Gonococcemia (arthritis-dermatosis syndrome, disseminated gonococcal infection), Gonorrhea (clap) Gram-negative folliculitis, Gram-negative toe web infection, Granuloma inguinale (Donovanosis, granuloma genitoinguinale, granuloma inguinale tropicum, granuloma venereum, granuloma venereum genitoinguinale, lupoid form of groin ulceration, serpiginous ulceration of the groin, ulcerating granuloma of the pudendum, ulcerating sclerosing granuloma), Green nail syndrome, Group JK Corynebacterium sepsis, Haemophi/us influenzae cellulitis, Helicobacter cellulitis, Hospital furunculosis, Hot tub folliculitis (Pseudomonas aeruginosa folliculitis), Human granulocytotropic anaplasmosis, Human monocytotropic ehrlichiosis, Impetigo contagiosa, Japanese spotted fever, Leptospirosis (Fort Bragg fever, pretibial fever, Weil's disease), Listeriosis, Ludwig's angina, Lupoid sycosis, Lyme disease (Afzelius' disease, Lyme borreliosis), Lymphogranuloma venereum (climatic bubo, Durand-Nicolas-Favre disease, lymphogranuloma inguinale, poradenitis inguinale, strumous bubo), Malakoplakia (malacoplakia), Mediterranean spotted fever (Boutonneuse fever), Melioidosis (Whitmore's disease), Meningococcemia, Missouri Lyme disease, Mycoplasma infection, Necrotizing fasciitis (flesh-eating bacteria syndrome), Neonatal toxic shock-like exanthematous disease, Nocardiosis, Noma neonatorum, North Asian tick typhus, Ophthalmia neonatorum, Oroya fever (Carrion's disease), Pasteurellosis, Perianal cellulitis (perineal dermatitis, streptococcal perianal disease), Periapical abscess, Pinta, Pitted keratolysis (keratolysis plantare sulcatum, keratoma plantare sulcatum, ringed keratolysis), Plague, Primary gonococcal dermatitis, Pseudomonal pyoderma, Pseudomonas hot-foot syndrome, Pyogenic paronychia, Pyomyositis, Q fever, Queensland tick typhus, Rat-bite fever, Recurrent toxin-mediated perineal erythema, Rhinoscleroma, Rickettsia aeschlimannii infection, Rickettsialpox, Rocky Mountain spotted fever, Saber shin (anterior tibial bowing), Saddle nose, Salmonellosis, Scarlet fever, Scrub typhus (Tsutsugamushi fever), Shigellosis, Staphylococcal scalded skin syndrome (pemphigus neonatorum, Ritter's disease), Streptococcal intertrigo, Superficial pustular folliculitis (impetigo of Bockhart, superficial folliculitis), Sycosis vulgaris (barber's itch, sycosis barbae), Syphilid, Syphilis (lues) Tick-borne lymphadenopathy, Toxic shock syndrome (streptococcal toxic shock syndrome, streptococcal toxic shock-like syndrome, toxic streptococcal syndrome), Trench fever (five-day fever, quintan fever, urban trench fever), Tropical ulcer (Aden ulcer, jungle rot, Malabar ulcer, tropical phagedena), Tularemia (deer fly fever, Ohara's disease, Pahvant Valley plague, rabbit fever), Verruga peruana, Vibrio vulnificus infection, Yaws (bouba, frambOsie, parangi, pian), Aquarium granuloma (fish-tank granuloma, swimming-pool granuloma), Borderline lepromatous leprosy, Borderline leprosy, Borderline tuberculoid, leprosy, Buruli ulcer (Bairnsdale ulcer, Searl ulcer, Searle's ulcer), Erythema induratum (Bazin disease), Histoid leprosy, Lepromatous leprosy, Leprosy (Hansen's disease), Lichen scrofulosorum (tuberculosis cutis lichenoides), Lupus vulgaris (tuberculosis luposa), Miliary tuberculosis (disseminated tuberculosis, tuberculosis cutis acuta generalisata, tuberculosis cutis disseminata), Mycobacterium avium-intracel ulare complex infection, Mycobacterium haemophi/um infection, Mycobacterium kansasii infection, Papulonecrotic tuberculid, Primary inoculation tuberculosis (cutaneous primary complex, primary tuberculous complex, Tuberculous chancre), Rapid-growing Mycobacterium infection, Scrofuloderma (Tuberculosis cutis colliquativa), Tuberculosis cutis orificialis (acute tuberculous ulcer, orificial tuberculosis), Tuberculosis verrucosa cutis (lupus verrucosus, prosector's wart, warty tuberculosis), Tuberculous cellulitis, Tuberculous gumma (metastatic tuberculous abscess, metastatic tuberculous ulcer), Tuberculoid leprosy, and sexually transmitted diseases caused by bacteria. Non-limiting examples of sexually transmitted diseases that comprise a microbial infection include Chancroid, Chlamydia, Gonorrhea, Lymphogranuloma Venereum, Mycoplasma Genitalium, Nongonococcal Urethritis, Pelvic Inflammatory Disease, Syphilis, vaginitis, bacterial vaginitis, yeast vaginitis, yeast infection.

In another embodiment, the microbial infection is a fungal infection. Non-limiting examples of infectious fungi causing fungal infections that are contemplated for use with the combinatorial therapeutic compositions and methods described herein include, but are not limited to: Candida spp.; Cryptococcus spp.; Aspergillus spp.; Microsporum spp.; Trichophyton spp.; Epidermophyton spp.; Trichosporon spp.; Tinea versicolor; Tinea barbae; Tinea corporis; Tinea cruris; Tinea manuum; Tinea pedis; Tinea unguium; Tinea faciei; Tinea imbricate; Tinea incognito; Epidermophyton floccosum; Microsporum canis; Microsporum audouinii; Trichophyton interdigitale; Trichophyton mentagrophytes; Trichophyton tonsurans; Trichophyton schoenleini; Trichophyton rubrum; Hortaea werneckii; Piedraia hortae; Malasserzia furfur; Coccidioides immitis; Coccidioides posadasii; Histoplasma capsulatum; Histoplasma duboisii; Lacazia loboi; Paracoccidioides brasiliensis; Blastomyces dermatitidis; Sporothrix schenckii; Penicillium marneffei; Candida albicans; Candida glabrata; Candida tropicalis; Candida lusitaniae; Candida jirovecii; Exophiala jeanselmei; Fonsecaea pedrosoi; Fonsecasea compacta; Phialophora verrucosa; Geotrichum candidum; Pseudallescheria boydii; Rhizopus oryzae; Muco indicus; Absidia corymbifera; Synceplasastrum racemosum; Basidiobolus ranarum; Conidiobolus coronatus; Conidiobolus incongruous; Cryptococcus neoformans; Enterocytozoan bieneusi; Encephalitozoon intestinalis; and Rhinosporidium seeberi.

Non-limiting examples of disorders/diseases caused by fungal infections or toxins produced during fungal infections, and for which the compositions and methods described herein are applicable in various aspects and embodiments, include, but are not limited to, infection of a surface wound or burn; infection of a mucosal surface; respiratory infection; infections of the eyes, ears, nose, or throat; or infection of an intestinal pathogen. In other embodiments, the fungal infection is an infection of soft tissue or skin, such as a superficial mycosis; a cutaneous mycosis; a subcutaneous mycosis; a vaginal mycosis; a systemic mycosis; or is an infected wound or burn.

Other medically relevant microorganisms have been described extensively in the literature, e.g., see C. G. A Thomas, Medical Microbiology, Bailliere Tindall, Great Britain 1983, the entire contents of which is hereby incorporated by reference. Each of the foregoing lists is illustrative and is not intended to be limiting.

### Viral Infections

Immunostimulatory complexes or RNA duplexes described herein, or or pharmaceutical formulations comprising them can be used to treat a viral infection.

In one embodiment, a subject is diagnosed with having a viral infection prior to administration of an immunostimulatory complex or RNA duplex or any composition described herein. In another embodiment, the treatment method comprises a step of diagnosing the subject as having a viral infection. In another embodiment, prior to administering, the treatment method comprises a step of receiving results of an assay that diagnoses the subject as having a viral infection or as being at risk of having a viral infection.

In one aspect, described herein is a method of inducing an anti-viral response in a subject, the method comprising administering to a subject an immunostimulatory complex or RNA duplex as described herein.

In another aspect, described herein is a method of treating a viral infection in a subject by administering to the subject an immunostimulatory complex or RNA duplex as described herein.

In some embodiments, the viral infection is an infection of a tissue selected from the group consisting of central nervous system tissue, eye tissue, upper respiratory system tissue, lower respiratory system tissue, lung tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.

Non-limiting examples of viral infections include respiratory infections of the nose, throat, upper airways, and lungs such as influenza, pneumonia, coronavirus, SARS, COVID 19, bronchiolitis, and laryngotracheobronchitis; gastrointestinal infections such as gastroenteritis, rotavirus, norovirus; liver infections such as hepatitis; nervous system infections such as rabies, West Nile virus, encephalitis, meningitis, and polio; skin infections such as warts, blemishes, and chickenpox; placental and fetal viral infections such as Zika virus, Rubella virus, and cytomegalovirus; enteroviruses, coxsackieviruses; echoviruses, chikungunya virus, Crimean-Congo hemorrhagic fever virus, Japanese encephalitis virus, Rift Valley Fever virus, Ross River virus, louping ill virus, John Cunningham virus, measles virus, lymphocytic choriomeningitis virus, arbovirus, rhinovirus, parainfluenza virus, respiratory syncytial virus, herpes simplex virus, herpes simplex type 1, herpes simplex type 2, human herpesvirus 6, adenovirus, cytomegalovirus, Epstein-Barr virus, mumps virus, influenza virus type A, influenza virus type B, coronavirus, SARS coronavirus, SARS-CoV-2 virus, coxsackie A virus, coxsackie B virus, poliovirus, HTLV-1, hepatitis virus types A, B, C, D, and E, varicella zoster virus, smallpox virus, molluscum contagiosum, human papillomavirus, parvovirus B19, rubella virus, human immunodeficiency virus, rotavirus, norovirus, astrovirus, ebola virus, Marburg virus, dengue virus (DENV), and Zika virus.

One aspect herein is a method of treating an influenza infection comprising administering to a subject having an influenza infection any of the immunostimulatory complexes described herein or any of the pharmaceutical compositions described herein. In one embodiment, the influenza infection is an influenza A infection, or an influenza B infection.

In one embodiment, a subject is diagnosed with having an influenza infection prior to administration of an immunostimulatory complex or RNA duplex or any composition described herein. In another embodiment, the method comprises a step of diagnosing the subject as having an influenza infection. In another embodiment, prior to administering, the method comprises a step of receiving results of an assay that diagnoses the subject as having an influenza infection or as being at risk of having an influenza infection.

One aspect herein is a method of treating a coronavirus infection in a subject comprising administering to a subject having a coronavirus infection any of the immunostimulatory complexes described herein or any of the pharmaceutical compositions described herein. In one embodiment, the coronavirus infection is a SARS-CoV-2 or variants thereof (e.g., delta, lambda, etc.), SARS-CoV-1, MERS-CoV, or HCoV-NL63 infection.

In one embodiment, a subject is diagnosed as having a coronavirus infection prior to administration of an immunostimulatory complex or RNA duplex or any composition described herein. In another embodiment, the method comprises a step of diagnosing the subject as having a coronavirus infection. In another embodiment, prior to administering, the method comprises a step of receiving results of an assay that diagnoses the subject as having a coronavirus infection or as being at risk of having a coronavirus infection.

In one embodiment, the subject is at risk of developing a viral infection. Risk factors for having or developing a viral infection include exposure to the virus, exposure or contact with a subject infected with a virus, exposure to contaminated surfaces contacted with a virus, contact with a biological sample or bodily fluid from a subject infected by a virus, sexual intercourse with a subject infected by a virus, needle sharing, blood transfusions, drug use, and any other risk factor known in the art to transmit a virus from one subject to another. Risk factors for a subject can be evaluated, e.g., by a skilled clinician or by the subject.

### Identification of Microbial Infections

The immunostimulatory complex or RNA duplexes or any composition described herein can be used to treat a microbial infection.

In one embodiment, a subject is diagnosed with having a microbial infection prior to administration of an immunostimulatory complex or RNA duplex or any composition described herein. In another embodiment, the method comprises a step of diagnosing the subject as having a microbial infection. In another embodiment, prior to administering, the method comprises a step of receiving results of an assay that diagnoses the subject as having a viral infection or as being at risk of having a microbial infection.

There are various tests known to those skilled in the art that are performed in a laboratory to establish or confirm the diagnosis of a microbial infection, as well as to identify the causative microbial species. Common viral infections can be diagnosed based on symptoms, e.g., measles, rubella, chicken pox. The symptoms associated with viral infection vary depending on the type of virus. For example, for an upper respiratory viral infection symptoms include but are not limited to coughing; shortness of breath; fever; and malaise.

For infections that occur in epidemics (e.g., COVID 19 and influenza), the presence of other similar cases may help doctors identify a particular infection. Laboratory diagnosis is important for distinguishing between different viruses that cause similar symptoms, such as SARS-CoV2 (COVID-19) and influenza.

Culturing of microbial species, with antimicrobial sensitivity testing is considered the gold standard laboratory test for some microbes. Skin or mucosal samples can be collected in the following ways: 1) dry sterile cotton-tip swab rubbed on the infection site, 2) moist swab taken from a mucosal surface, such as inside the mouth; 3) aspiration of fluid/pus from a skin lesion using a needle and syringe; and 4) skin biopsy: a small sample of skin removed under local anesthetic. Culturing of, e.g., bacteria is most commonly done by brushing the skin swab on sheep blood agar plates and exposing them to different conditions. The species of microbe that grow depend on the medium used to culture the specimen, the temperature for incubation, and the amount of oxygen available. For example, an obligate aerobe can only grow in the presence of oxygen, while an obligate anaerobe cannot grow at all in the presence of oxygen.

Blood tests require a sample of blood accessed by a needle from a vein. Non-limiting examples of tests for microbial infections include: 1) full blood count, infection often raises the white cell count with increased neutrophils (neutrophilia); 2) C-reactive protein (CRP), CRP is often elevated >50 in serious infections; 3) procalcitonin, a marker of generalized sepsis due to bacterial infection, 3) serology, tests 10 days apart to determine immune response to a particular organism; 4) Rapid Plasma Reagin (RPR) test, if syphilis is suspected; and 4) blood culture to detect if high fever >100.4° F. Blood tests can be performed to identify antibodies generated in the presence of a microbial infection.

Polymerase chain reaction (PCR) involves isolating and amplifying lengths of microbial DNA from a sample of skin, blood, or other tissue. The DNA of the sample is compared to DNA from known organisms, thus identifying the species.

### Treatments for Microbial Infections

A number of medications for the treatment of an infection (e.g., a bacterial or viral infection) have been developed. Treatments for infections can include, for example, antibiotics and antiviral medications administered following infection.

The term “therapeutic agent” is art-recognized and refers to any biologic or chemical moiety that is a biologically, physiologically, or pharmacologically active substance that acts locally or systemically in a subject. Examples of therapeutic agents, also referred to as “drugs”, are described in well-known literature references such as the Merck Index, the Physicians' Desk Reference, and The Pharmacological Basis of Therapeutics, and they include, without limitation, medications; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent may be used which are capable of being released from the subject composition into adjacent tissues or fluids upon administration to a subject.

Exemplary therapeutic agents and vaccines for the prevention and treatment of infections include but are not limited to penicillin, ceftriaxone, azithromycin, amoxicillin, doxycycline, cephalexin, ciprofloxacin, clindamycin, metronidazole, azithromycin, sulfamethoxazole, trimethoprim, meningococcal polysaccharide vaccine, tetanus toxoid, cholera vaccine, typhoid vaccine, pneumococcal 7-valent vaccine, pneumococcal 13-valent vaccine, pneumococcal 23-valent vaccine, haemophilus b conjugate, anthrax vaccine, imunovir, indinavir, inosine, lopinavir, lovaride, maravirox, nevirapine, nucleoside analogues, oseltamivir, penciclovir, rimantidine, pyrimidine, saquinavir, stavudine, tenofovir, trizivir, tromantadine, truvada, valaciclovir, ciramidine, zanamivir, zidovudine, MMR vaccine, DTaP vaccine, hepatitis vaccines, Hib vaccine, HPV vaccine, influenza vaccine, polio vaccine, rotavirus vaccine, shingles vaccine, Tdap vaccine, tetanus vaccine, fluconazole, ketoconazole, amphotericin B, and sulfadoxine/pyrimethamine. Additional non-limiting examples include Abacavir, Acyclovir (Aciclovir), Adefovir, Amantadine, Ampligen, Amprenavir (Agenerase), Amodiaquine, Apilimod, Arbidol, Atazanavir, Atripla, Balavir, Baloxavir marboxil (Xofluza®), Biktarvy Boceprevir (Victrelis®), Cidofovir, Clofazimine, Clomifene, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Favipiravir, Fenofibrate, Fomivirsen, Fosamprenavir, Foscarnet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Lamivudine, Letermovir (Prevymis®), Lopinavir, Loviride, Mannose Binding Lectin, Maraviroc, Methisazone, Moroxydine, Nafamostat, Nelfinavir, Nevirapine, Nexavir®, Nilotinib, Nitazoxanide, Norvir, Nucleoside analogues, Oseltamivir (Tamiflu®), Pazopanib, Peginterferon alfa-2a, Peginterferon alfa-2b, Penciclovir, Peramivir (Rapivab®), Pleconaril, Podophyllotoxin, Protease inhibitor (pharmacology), Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Telaprevir, Telbivudine (Tyzeka®), Tenofovir alafenamide, Tenofovir disoproxil, Tenofovir, Toremifene, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza®), and Zidovudine.

In some embodiments of any ofthe aspects, the immunostimulatory complex or RNA duplexes described herein are used as a monotherapy.

In another embodiment of any ofthe aspects, the immunostimulatory complex, RNA duplex(e) or compositions described herein can be used in combination with other known compositions and therapies for an interferon-mediated disease (e.g., autoimmune disease, infection, or cancer). The immunostimulatory complex or RNA duplexes described herein can be e.g., in admixture with an antiviral therapeutic or administered as a therapeutic regimen for the treatment of an interferon-mediated disease.

One aspect described herein is a method of increasing the efficacy of an anti-viral therapeutic comprising administering to a subject in need thereof any of the immunostimulatory complexes or RNA duplexes, or compositions thereof as described herein in combination with the anti-viral therapeutic. In one embodiment, the efficacy of the anti-viral therapeutic is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control is an otherwise identical subject that is not administered any of the immunostimulatory complexes or RNA duplexes, or composition thereof, or is not administered the same dose of any of the immunostimulatory complexes or RNA duplexes, or composition thereof, or is not administered the same combination of any of the immunostimulatory complexes or RNA duplexes, or composition thereof.

Administered “in combination,” as used herein, means that two (or more) different treatments are delivered to the subject during the course of the subject's affliction with the disorder, e.g., the two or more treatments are delivered after the subject has been diagnosed with the disorder (a respiratory disease) and before the disorder has been cured or eliminated or treatment has ceased for other reasons. Non-limiting examples of treatments that can be used in combination with the compositions provided herein include Abacavir, Acyclovir (Aciclovir), Adefovir, Amantadine, Ampligen, Amprenavir (Agenerase), Amodiaquine, Apilimod, Arbidol, Atazanavir, Atripla, Atovaquone, Balavir, Baloxavir marboxil (Xofluza®), Biktarvy Boceprevir (Victrelis®), Cidofovir, Clofazimine, Clomifene, Clofazamine, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Favipiravir, Fenofibrate, Fomivirsen, Fosamprenavir, Foscamet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Ivermectin, Lamivudine, Lasalocid, Letermovir (Prevymis®), Lopinavir, Loviride, Mannose Binding Lectin, Maraviroc, Methisazone, Moroxydine, Nafamostat, Nelfinavir, Nevirapine, Nexavir®, Nilotinib, Nitazoxanide, Norvir, Nucleoside analogues, Oseltamivir (Tamiflu®), Pazopanib, Peginterferon alfa-2a, Peginterferon alfa-2b, Penciclovir, Peramivir (Rapivab®), Pleconaril, Podophyllotoxin, Protease inhibitor (pharmacology), Pyonaridine, Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Tafenoquine, Telaprevir, Telbivudine (Tyzeka®), Tenofovir alafenamide, Tenofovir disoproxil, Tenofovir, Toremifene, Tipranavir, Trifluridine, Trizivir, Tromantadine, Truvada, Valaciclovir (Valtrex), Valganciclovir, Vermurafenib, Venetoclax, Vicriviroc, Vidarabine, Viramidine, Zalcitabine, Zanamivir (Relenza®), and Zidovudine.

In some embodiments, the immunostimulatory complex or RNA duplex and the at least one antiviral therapeutic are administered at substantially the same time.

In some embodiments, the immunostimulatory complex or RNA duplex and the at least one antiviral therapeutic are administered at different time points.

In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The compositions described herein and the at least one additional therapy can be administered simultaneously, in the same or in separate compositions, or sequentially. For sequential administration, the composition described herein can be administered first, and the additional composition can be administered second, or the order of administration can be reversed. The composition and/or other therapeutic compositions, procedures or modalities can be administered during periods of active disorder, or during a period of remission or less active disease. The composition can be administered before another treatment, concurrently with the treatment, post-treatment, or during remission of the disorder.

When administered in combination, the complex or RNA duplex or composition and the additional agent or composition (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of a respiratory disease) is lower (e.g., at least 20%, at least 30%, at least 40%, or at least 50% lower) than the amount or dosage of each agent individually required to achieve the same therapeutic effect.

One aspect described herein is a method of vaccinating comprising administering to a subject in need thereof any of the immunostimulatory complexes or RNA duplexes, or composition thereof as described herein.

Another aspect described herein is a method of increasing the efficacy of a vaccine comprising administering to a subject in need thereof any of the immunostimulatory complexes or RNA duplexes, or composition thereof as described herein. In one embodiment, the efficacy of the vaccine is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control is an otherwise identical subject that is not administered any of the immunostimulatory complexes or RNA duplexes, or composition thereof, or is not administered the same dose of any of the immunostimulatory complexes or RNA duplexes, or composition thereof, or is not administered the same combination of any of the immunostimulatory complexes or RNA duplexes, or composition thereof.

A vaccine composition as described herein can be used, for example, to protect or treat a subject against disease. The terms “immunize” and “vaccinate” tend to be used interchangeably in the field. However, in reference to the administration of the vaccine compositions as described herein to provide protection against disease, e.g., infectious disease caused by a pathogen that expresses the antigen, it should be understood that the term “immunize” refers to the passive protection conferred by the administered vaccine composition.

In one embodiment, administration of the immunostimulatory complex, e.g., a concatamer, or RNA duplex reduces the viral titer or viral load in the cell or cell populations. In one embodiment, the viral titer or viral load is decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control is an otherwise identical subject that is not administered any of the immunostimulatory complexes or RNA duplexes, or composition thereof, or is not administered the same dose of any of the immunostimulatory complexes or RNA duplexes, or composition thereof, or is not administered the same combination of any of the immunostimulatory complexes or RNA duplexes, or composition thereof. One skilled in the art can assess viral titer or vial load using standard techniques, e.g., using assay described herein below.

### Administration, Dosage, and Efficacy

The immunostimulatory complex or RNA duplex, pharmaceutical composition, or vaccine compositions described herein can be formulated, dosed, and administered in a fashion consistent with good medical practice. Factors for consideration in this context include the particular disorder being treated, the particular subject being treated, the clinical condition of the individual subject, the cause of the disorder, the site of delivery of the vaccine composition, the method of administration, the scheduling of administration, and other factors known to medical practitioners.

The therapeutic formulations to be used for in vivo administration, such as parenteral administration, in the methods described herein can be sterile, which is readily accomplished by filtration through sterile filtration membranes, or other methods known to those of skill in the art.

The immunostimulatory complex or RNA duplexes and compositions thereof as described herein can be administered to a subject in need thereof by any appropriate route which results in an effective treatment in the subject. As used herein, the terms “administering,” and “introducing” are used interchangeably and refer to the placement of an immunostimulatory complex, RNA duplex or composition comprising them into a subject by a method or route which results in at least partial localization of such compositions at a desired site, such as a site of infection, such that a desired effect(s) is produced. An immunostimulatory complex, RNA duplex or composition comprising them can be administered to a subject by any mode of administration that delivers the composition systemically or to a desired surface or target, and can include, but is not limited to, injection, infusion, instillation, and inhalation administration. To the extent that an immunostimulatory complex, RNA duplex or composition comprising them can be protected from inactivation in the gut, oral administration forms are also contemplated. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of a therapeutic agent other than directly into a target site, tissue, or organ, such that it enters the subject's circulatory system and, thus, is subject to metabolism and other like processes. In other embodiments, the immunostimulatory complex, RNA duplex or composition comprising them is administered locally, e.g., by direct injections, when the disorder or location of the infection permits, and the injections can be repeated periodically.

In some embodiments, the compositions described herein are administered by aerosol administration, nebulizer administration, or tracheal lavage administration. In some embodiments, the composition is formulated for intravenous, intramuscular, intraperitoneal, subcutaneous, or intrathecal administration.

The term “effective amount” as used herein refers to the amount of an immunostimulatory complex or RNA duplex composition needed to alleviate or prevent at least one or more symptom of an infection, disease or disorder, and relates to a sufficient amount of pharmacological composition to provide the desired effect, e.g., reduce the level of pathogenic microorganisms at a site of infection, reduce pathology, or any symptom associated with or caused by the pathogenic microorganism. The term “therapeutically effective amount” therefore refers to an amount of an immunostimulatory complex or RNA duplex composition as described herein using the methods as disclosed herein, that is sufficient to effect a particular effect when administered to a typical subject. An effective amount as used herein would also include an amount sufficient to delay the development of a symptom of the disease, alter the course of a symptom disease (for example, but not limited to, slow the progression of a symptom of the disease), or reverse a symptom of the disease. Thus, it is not possible to specify the exact “effective amount.” However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the antigen or fragment thereof), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The immunostimulatory complexes, RNA duplexes orpharmaceutical compositions described herein can be formulated, in some embodiments, with one or more additional therapeutic agents currently used to prevent or treat an infection, for example. The effective amount of such other agents depends on the amount of immunostimulatory complex or RNA duplex in the formulation, the type of disorder or treatment, and other factors discussed above. These can be used in the same dosages and with administration routes as described elsewhere herein. In some embodiments, the amount of an additional therapeutic agent or the frequency of its administration needed for therapeutic effect can be reduced when administered in conjunction with an immunostimulatory complex or RNA duplex as described herein. In such embodiments, the amount can be reduced by 5%, 10%, 15%, 20% 25%, 30%, 35%, 40%, 45%, 50% or more relative to administration of the additional therapeutic agent alone.

The dosage ranges for the immunostimulatory complexes, RNA duplexes, or pharmaceutical compositions as described herein depend upon the potency, and encompass amounts large enough to produce the desired effect. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 100 mg/kg body weight. In some embodiments, the dose range is from 5 g/kg body weight to 100 g/kg body weight. Alternatively, the dose range can be titrated to maintain serum levels between 1 g/mL and 1000 g/mL. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 7.5 mg/kg, 10 mg/kg, 15 mg/kg, 20 mg/kg, 25 mg/kg, 30 mg/kg, 40 mg/kg, 50 mg/kg, or more. These doses can be administered by one or more separate administrations, or by continuous infusion. For repeated administrations over several days or longer, depending on the condition, the treatment is sustained until, for example, the infection is treated, as measured by the methods described above or known in the art. However, other dosage regimens can be useful.

The duration of a therapy using the methods described herein will continue for as long as medically indicated or until a desired therapeutic effect (e.g., those described herein) is achieved. In certain embodiments, the administration of the composition described herein is continued for 1 month, 2 months, 4 months, 6 months, 8 months, 10 months, 1 year, 2 years, 3 years, 4 years, 5 years, 10 years, 20 years, or for a period of years up to the lifetime of the subject.

As will be appreciated by one of skill in the art, appropriate dosing regimens for a given composition can comprise a single administration/immunization or multiple ones. Subsequent doses may be given repeatedly at time periods, for example, about two weeks or greater up through the entirety of a subject's life, e.g., to provide a sustained preventative effect. Subsequent doses can be spaced, for example, about two weeks, about three weeks, about four weeks, about one month, about two months, about three months, about four months, about five months, about six months, about seven months, about eight months, about nine months, about ten months, about eleven months, or about one year after a primary immunization.

The precise dose to be employed in the formulation will also depend on the route of administration and should be decided according to the judgment of the practitioner and each patient's circumstances. Ultimately, the practitioner or physician will decide the amount of the immunostimulatory complex or RNA duplex or composition thereof to administer to particular subjects.

In some embodiments of these methods and all such methods described herein, the immunostimulatory complex or RNA duplex or composition thereof is administered in an amount effective to provide short-term protection against an infection or to treat an infection. In some embodiments, the infection is a viral infection. As used herein, “short-term protection” refers to protection from an infection, such as a malarial infection, lasting at least about 2 weeks, at least about 1 month, at least about 6 weeks, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. Such protection can involve repeated dosing.

In some embodiments of these methods and all such methods described herein, the immunostimulatory complex or RNA duplex or composition thereof is administered in an amount effective to provide protection against an infection or to alleviate a symptom of a persistent infection.

“Alleviating a symptom of a persistent infection” is ameliorating any condition or symptom associated with the persistent infection. Alternatively, alleviating a symptom of a persistent infection can involve reducing the infectious microbial (such as viral, bacterial, fungal or parasitic) load in the subject relative to such load in an untreated control. As compared with an equivalent untreated control, such reduction or degree of prevention is at least 5%, 10%, 20%, 40%, 50%, 60%, 80%, 90%, 95%, or 100% as measured by any standard technique. Desirably, the persistent infection is completely cleared as detected by any standard method known in the art, in which case the persistent infection is considered to have been treated.

A patient who is being treated for a persistent infection is one who a medical practitioner has diagnosed as having such a condition. Diagnosis may be by any suitable means. Diagnosis and monitoring may involve, for example, detecting the level of microbial load in a biological sample (for example, a tissue biopsy, blood test, or urine test), detecting the level of a surrogate marker of the microbial infection in a biological sample, detecting symptoms associated with persistent infections, or detecting immune cells involved in the immune response typical of persistent infections (for example, detection of antigen specific T cells that are anergic and/or functionally impaired). A patient in whom the development of a persistent infection is being prevented may or may not have received such a diagnosis. One in the art will understand that these patients may have been subjected to the same standard tests as described above or may have been identified, without examination, as one at high risk due to the presence of one or more risk factors (such as family history or exposure to infectious agent).

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

The technology provided herein can further be defined by the following numbered paragraphs.
1. An immunostimulatory complex comprising a concatamer of oligonucleotide duplexes, wherein each duplex comprises an oligonucleotide strand having the structure 5′-C—N₁₆-GGG-3′ and an oligonucleotide strand having the structure 5′-C—N′₁₆-GGG-3′, wherein:
   N and N′ are each any of G, A, U and C;
   N₁₆ is complementary to N′₁₆; and
   duplexes in the concatamer are joined by Hoogsteen base pairing between 3′-GG overhanging dinucleotides on each duplex.
2. The immunostimulatory complex of paragraph 1, wherein the concatamer is a dimer of oligonucleotide duplexes.
3. The immunostimulatory complex of any of the preceding paragraphs, wherein the concatamer comprises three or more of the oligonucleotide duplexes.
4. The immunostimulatory complex of any of the preceding paragraphs, wherein one or both oligonucleotide strands of each duplex comprise(s) a 5′-terminal monophosphate, diphosphate, triphosphate or hydroxyl group.
5. The immunostimulatory complex of any of the preceding paragraphs, wherein the oligonucleotide duplexes comprise double stranded RNA.
6. The immunostimulatory complex of any of the preceding paragraphs, wherein the concatamer induces interferon (IFN) production in a cell
7. The immunostimulatory complex of any of the preceding paragraphs, wherein the IFN production is type I IFN production.
8. The immunostimulatory complex of any of the preceding paragraphs, wherein the concatamer activates the RIG-I-IRF3 pathway
9. The immunostimulatory complex of any of the preceding paragraphs, wherein the concatamer reduces a viral titer or viral load in a cell or population of cells.
10. An immunostimulatory complex comprising at least first and second RNA duplexes, each duplex comprising:
   a first strand comprising, from the 5′ terminus, the sequence 5′-C—N₁₉-3′, and
   a second strand comprising, at the 3′ terminus, the sequence 5′-N′₁₉-GGG-3′,
wherein:
   N and N′ are any of C, A, G, and U;
   N and N′ are complementary;
the 3′ terminal GG dinucleotide of the second strand forms a 3′ GG dinucleotide overhang;
the first duplex is complexed with the at least second duplex via Hoogsteen base pairing between the 3′ GG overhang on each duplex; and
   the first strand, at the 5′ terminus, does not comprise the sequence 5′-CUGA-3′.
11. The immunostimulatory complex of any of the preceding paragraphs, wherein one or both oligonucleotide strands of each duplex comprise(s) a 5′-terminal monophosphate, diphosphate, triphosphate or hydroxyl group.
12. The immunostimulatory complex of any of the preceding paragraphs, wherein the RNA duplexes comprise double stranded RNA.
13. The immunostimulatory complex of any ofthe preceding paragraphs, wherein the RNA duplexes comprise one or more DNA nucleotides at the duplex end opposite the 5′-C.
14. The immunostimulatory complex of any ofthe preceding paragraphs, wherein the RNA duplexes comprise comprises a blunt end, a 5′ overhang or a 3′ overhang on the end opposite the 5′-C.
15. The immunostimulatory complex of any of the preceding paragraphs, wherein the complex induces interferon (IFN) production in a cell.
16. The immunostimulatory complex of any of the preceding paragraphss, wherein the IFN production is type I IFN production.
17. The immunostimulatory complex of any of the preceding paragraphs, wherein the complex activates the RIG-I-IRF3 pathway.
18. The immunostimulatory complex of any of the preceding paragraphs, wherein the complex reduces a viral titer or viral load in a cell or population of cells.
19. A pharmaceutical composition comprising the immunostimulatory complex of any of the preceding paragraphs.
20. The pharmaceutical composition of any of the preceding paragraphs, which further comprises a pharmaceutically acceptable carrier.
21. The composition of any of the preceding paragraphs, wherein the composition is formulated for airway administration.
22. The composition of any of the preceding paragraphs, wherein the composition is formulated for aerosol administration, nebulizer administration, or tracheal lavage administration.
23. A composition comprising an immunostimulatory complex of any of the preceding paragraphs or a pharmaceutical composition of any of the preceding paragraphs and a vaccine.
24. A composition comprising an immunostimulatory complex of any of the preceding paragraphs or a pharmaceutical composition of any of the preceding paragraphs and a nanoparticle.
25. A nanoparticle comprising an immunostimulatory complex of any of the preceding paragraphs or a pharmaceutical composition of any of the preceding paragraphs.
26. A method of inducing an anti-viral response in a subject, the method comprising administering to a subject in need thereof an immunostimulatory complex of any of the preceding paragraphs or a pharmaceutical composition of any of the preceding paragraphs.
27. A method of treating or preventing a viral infection in a subject, the method comprising administering to a subject in need thereof an immunostimulatory complex of any of the preceding paragraphs or a pharmaceutical composition of any of the preceding paragraphs.
28. The method of any of the preceding paragraphs, wherein the subject in need thereof has a viral infection, or is at risk of having a viral infection.
29. The method of any of the preceding paragraphs, further comprising, prior to administering, a step of diagnosing the subject as having a viral infection or being at risk of having a viral infection.
30. The method of any of the preceding paragraphs, further comprising, prior to administering, a step of receiving results of an assay that diagnoses the subject as having a viral infection or as being at risk of having a viral infection.
31. The method of any of the preceding paragraphs, wherein the viral infection is caused by a virus selected from the group consisting of: John Cunningham virus, measles virus, Lymphocytic choriomeningitis virus, arbovirus, rabies virus, rhinovirus, parainfluenza virus, respiratory syncytial virus, herpes simplex virus, herpes simplex type 1, herpes simplex type 2, human herpesvirus 6, adenovirus, cytomegalovirus, Epstein-Barr virus, mumps virus, influenza virus type A, influenza virus type B, coronavirus, SARS coronavirus, SARS-CoV-2 virus, coxsackie A virus, coxsackie B virus, poliovirus, HTLV-1, hepatitis virus types A, B, C, D, and E, varicella zoster virus, smallpox virus, molluscum contagiosum, human papillomavirus, parvovirus B19, rubella virus, human immunodeficiency virus, rotavirus, norovirus, astrovirus, ebola virus, Marburg virus, dengue virus (DENV), and Zika virus.
32. The method of any of the preceding paragraphs, wherein the viral infection is an infection of a tissue selected from the group consisting of central nervous system tissue, eye tissue, upper respiratory system tissue, lower respiratory system tissue, lung tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.
33. The method of any ofthe preceding paragraphs, wherein the administration is systemic.
34. The method of any of the preceding paragraphs, wherein the administration is local at a site of viral infection.
35. The method of any of the preceding paragraphs, further comprising administering at least one additional therapeutic.
36. The method of any of the preceding paragraphs, wherein the at least one additional therapeutic is an anti-viral therapeutic.
37. A method of treating an influenza infection in a subject, the method comprising administering to a subject having an influenza infection an immunostimulatory complex of any of the preceding paragraphs or a pharmaceutical composition of any of the preceding paragraphs.
38. The method of any of the preceding paragraphs, wherein the influenza infection is an influenza A infection, or an influenza B infection.
39. The method of any of the preceding paragraphs, further comprising administering at least one additional anti-viral therapeutic.
40. A method of treating a coronavirus disease in a subject, the method comprising administering to a subject having a coronavirus infection an immunostimulatory complex of any of the preceding paragraphs or a pharmaceutical composition of any of the preceding paragraphs.
41. The method of any of the preceding paragraphs, wherein the coronavirus disease is COVID-19.
42. The method of any of the preceding paragraphs, further comprising administering at least one additional anti-viral therapeutic.
43. A method of inducing interferon (IFN) production, the method comprising administering to a subject in need thereof an immunostimulatory complex of any of the preceding paragraphs or a pharmaceutical composition of any of the preceding paragraphs, whereby IFN production is increased following administration.
44. The method of any of the preceding paragraphs, wherein IFN production is the production of type I IFN, type II IFN, or type III IFN.
45. The method of any of the preceding paragraphs, wherein IFN production is the production of type I IFN.
46. An immunostimulatory RNA duplex having
   a) a first strand having from 5′ to 3′ a GNNN sequence flanked by at least 22 nucleotides on each side; and
   b) a second strand having from 5′-3′ a GGGC sequence flanked by at least 22 nucleotides on each side,
   wherein the first and second strands are complementary to each other.
47. The RNA duplex of any of the preceding paragraphs, wherein the first and/or second strand has a two nucleotide overhang at its 3′ end.
48. The RNA duplex of any of the preceding paragraphs, wherein the first and/or second strand have two DNA nucleosides at its 3′ end.
49. The RNA duplex of any of the preceding paragraphs, wherein the DNA nucleosides are thymidines.
50. The RNA duplex of any of the preceding paragraphs, wherein the first and/or second strand has a TT overhang at its 3′ end.
51. The RNA duplex of any of the preceding paragraphs, wherein the first and/or second strand comprises a 5′-terminal monophosphate, diphosphate, triphosphate or hydroxyl group.
52. The RNA duplex of any of the preceding paragraphs, wherein the RNA duplex is synthetic.
53. The RNA duplex of any ofthe preceding paragraphs, wherein the RNA duplex induces interferon (IFN) production in a cell.
54. The RNA duplex of any of the preceding paragraphs, wherein the IFN production is type I IFN production.
55. The RNA duplex of any of the preceding paragraphs, wherein the RNA duplex activates the RIG-I-IRF3 pathway.
56. The RNA duplex of any ofthe preceding paragraphs, wherein the RNA duplex reduces a viral titer or viral load in a cell or population of cells.
57. A synthetic RNA duplex having a first and second strand having a sequence selected from SEQ ID NO: 7-32.
58. A method of inducing an anti-viral response is a subject, the method comprising administering to a subject in need thereof an RNA duplex of any of the preceding paragraphs, or a synthetic RNA duplex of any of the preceding paragraphs.
59. A method of treating a viral infection in a subject, the method comprising administering to a subject in need thereof an RNA duplex of any of the preceding paragraphs, or a synthetic RNA duplex of any of the preceding paragraphs.
60. The method of any of the preceding paragraphs, wherein the subject in need thereof has a viral infection, or is at risk of having a viral infection.
61. The method of any of the preceding paragraphs, further comprising, prior to administering, a step of diagnosing the subject as having a viral infection or being at risk of having a viral infection.
62. The method of any of the preceding paragraphs, further comprising, prior to administering, a step of receiving results of an assay that diagnoses the subject as having a viral infection or as being at risk of having a viral infection.
63. The method of any of the preceding paragraphs, wherein the viral infection is caused by a virus selected from the group consisting of John Cunningham virus, measles virus, Lymphocytic choriomeningitis virus, arbovirus, rabies virus, rhinovirus, parainfluenza virus, respiratory syncytial virus, herpes simplex virus, herpes simplex type 1, herpes simplex type 2, human herpesvirus 6, adenovirus, cytomegalovirus, Epstein-Barr virus, mumps virus, influenza virus type A, influenza virus type B, coronavirus, SARS coronavirus, SARS-CoV-2 virus, coxsackie A virus, coxsackie B virus, poliovirus, HTLV-1, hepatitis virus types A, B, C, D, and E, varicella zoster virus, smallpox virus, molluscum contagiosum, human papillomavirus, parvovirus B19, rubella virus, human immunodeficiency virus, rotavirus, norovirus, astrovirus, ebola virus, Marburg virus, dengue virus (DENV), and Zika virus.
64. The method of any of the preceding paragraphs, wherein the viral infection is an infection of a tissue selected from the group consisting of central nervous system tissue, eye tissue, upper respiratory system tissue, lower respiratory system tissue, lung tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.
65. The method of any ofthe preceding paragraphs, wherein the administration is systemic.
66. The method of any of the preceding paragraphs, wherein the administration is local at a site of viral infection.
67. The method of any of the preceding paragraphs, further comprising administering at least one additional therapeutic.
68. The method of any of the preceding paragraphs, wherein the at least one additional therapeutic is an anti-viral therapeutic.
69. A method of treating an influenza infection in a subject, the method comprising administering to a subject having an influenza infection an RNA duplex of any of the preceding paragraphs, or a synthetic RNA duplex of any of the preceding paragraphs.
70. The method of any of the preceding paragraphs, wherein the influenza infection is an influenza A infection, or an influenza B infection.
71. The method of any of the preceding paragraphs, further comprising administering at least one additional anti-viral therapeutic.
72. A method of treating a coronavirus disease in a subject, the method comprising administering to a subject having a coronavirus disease an RNA duplex of any of the preceding paragraphs, or a synthetic RNA duplex of any of the preceding paragraphs.
73. The method of any of the preceding paragraphs, wherein the coronavirus disease is COVID-19.
74. The method of any of the preceding paragraphs, further comprising administering at least one additional anti-viral therapeutic.
75. A method of increasing the efficacy of an anti-viral therapeutic, the method comprising administering an RNA duplex of any of the preceding paragraphs, or a synthetic RNA duplex of any of the preceding paragraphs and at least one anti-viral therapeutic.
76. The method of any of the preceding paragraphs, wherein the anti-viral therapeutic is selected from the group consisting of: Abacavir, Acyclovir (Aciclovir), Adefovir, Amantadine, Ampligen, Amprenavir (Agenerase), Amodiaquine, Apilimod, Arbidol, Atazanavir, Atripla, Atovaquone, Balavir, Baloxavir marboxil (Xofluza®), Biktarvy Boceprevir (Victrelis®), Cidofovir, Clofazimine, Clomifene, Clofazamine, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Favipiravir, Fenofibrate, Fomivirsen, Fosamprenavir, Foscarnet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Ivermectin, Lamivudine, Lasalocid, Letermovir (Prevymis®), Lopinavir, Loviride, Mannose Binding Lectin, Maraviroc, Methisazone, Moroxydine, Nafamostat, Nelfinavir, Nevirapine, Nexavir®, Nilotinib, Nitazoxanide, Norvir, Nucleoside analogues, Oseltamivir (Tamiflu®), Pazopanib, Peginterferon alfa-2a, Peginterferon alfa-2b, Penciclovir, Peramivir (Rapivab®), Pleconaril, Podophyllotoxin, Protease inhibitor (pharmacology), Pyonaridine, Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Tafenoquine, Telaprevir, Telbivudine (Tyzeka®), Tenofovir alafenamide, Tenofovir disoproxil, Tenofovir, Toremifene, Tipranavir, Trifluridine, Trizivir, Tromantadine, Truvada, Valaciclovir (Valtrex), Valganciclovir, Vermurafenib, Venetoclax, Vicriviroc, Vidarabine, Viramidine, Zalcitabine, Zanamivir (Relenza®), and Zidovudine.
77. The method of any of the preceding paragraphs, wherein the RNA duplex and the at least one antiviral therapeutic are administered at substantially the same time.
78. The method of any of the preceding paragraphs, wherein the RNA duplex and the at least one antiviral therapeutic are administered at different time points.
79. A pharmaceutical composition comprising an RNA duplex of any of the preceding paragraphs, or a synthetic RNA duplex of any of the preceding paragraphs and a pharmaceutically acceptable carrier.
80. A pharmaceutical composition comprising an RNA duplex of any of the preceding paragraphs, or a synthetic RNA duplex of any of the preceding paragraphs and at least one anti-viral therapeutic.
81. The composition of any of the preceding paragraphs, wherein the composition is formulated for airway administration.
82. The composition of any of the preceding paragraphs, wherein the composition is formulated for aerosol administration, nebulizer administration, or tracheal lavage administration.
83. A method of inducing interferon (IFN) production, the method comprising administering to a subject in need thereof an RNA duplex of any of the preceding paragraphs, a synthetic RNA duplex of any of the preceding paragraphs, or a pharmaceutical composition of any of the preceding paragraphs, whereby IFN production is increased following administration.
84. The method of any of the preceding paragraphs, wherein IFN production is the production of type I IFN, type II IFN, or type III IFN.
85. The method of any of the preceding paragraphs, wherein IFN production is the production of type I IFN.
86. The method of any of the preceding paragraphs, wherein the type I IFN is IFN-α, IFN-β, IFN-ε, IFN-κ or IFN-ω.
87. The method of any of the preceding paragraphs, wherein increased IFN production increases cellular resistance to a viral infection.
88. A composition comprising an RNA duplex of any of the preceding paragraphs, or a synthetic RNA duplex of any of the preceding paragraphs and a vaccine.
89. A composition comprising an RNA duplex of any of the preceding paragraphs, or a synthetic RNA duplex of any of the preceding paragraphs and a nanoparticle.
90. A method of vaccinating, the method comprising administering to a subject in need thereof a composition of any of the preceding paragraphs.
91. A method of increasing the efficacy of a vaccine, the method comprising administering to a subject in need thereof an immunostimulatory complex of any of the preceding paragraphs, a composition of any of the preceding paragraphs, an RNA duplex of any of the preceding paragraphs, or a composition of any of the preceding paragraphs.
92. A method of preparing an RNAi molecule to promote degradation of a target RNA, the method comprising:
   a) identifying CCC trinucleotide repeats in the sequence of a target RNA;
   b) selecting a nucleotide sequence from 20 nucleotides to the upper limit for a dsRNA duplex that avoids a double-stranded RNA-activated protein kinase response, and lacks CCC repeats in a target RNA sequence as a candidate RNAi sequence);
   c) synthesizing an RNA molecule complementary to the sequence selected in step (b); and
   d) synthesizing an RNA molecule complementary to the RNA molecule synthesized in step (c),
   wherein combination of the RNA molecules synthesized in steps (c) and (d) produces an RNAi molecule that is less immunostimulatory than an RNAi molecule that targets the same target RNA but comprises a CCC trinucleotide repeat.
93. The method of paragraph 92, wherein the nucleotide sequence of b) is 20-29 nucleotides nucleotides in length.

### EXAMPLES

### Example 1

Described herein is a new class of immunostimulatory RNA dimers that potently induce production of type I interferon (IFN-I), and particularly IFN-β, in a wide range of human cell types via dimerization of GG overhangs that results in direct binding to RIG-I and activation of the RIG-I/IRF3 pathway. These duplex RNAs require a minimum of 20 base pairs, lack any sequence or structural characteristics of known immunostimulatory RNAs, and instead require a unique conserved sequence motif (sense strand: 5′-C, antisense strand: 3′-GGG) that mediates the formation of RNA dimers by Hoogsteen G-quadruplex base paring, i.e., end-to-end dimer self-assembly. The presence of terminal hydroxyl or monophosphate groups, blunt or overhanging ends, or terminal RNA or DNA bases did not affect their ability to induce IFN. Unlike previously described immunostimulatory siRNAs, their activity is independent of TLR7/8, but requires the RIG-I/IRF3 pathway that induces a more restricted antiviral response with a lower proinflammatory signature compared with immunostimulant poly(I:C). Immune stimulation mediated by these duplex RNAs results in broad spectrum inhibition of infections by many respiratory viruses with pandemic potential, including SARS-CoV-2, SARS-CoV, MERS-CoV, HCoV-NL63, and influenza A, as well as the common cold virus HCoV-NL63 in cell lines, human Lung Chips that mimic human lung pathophysiology, and in hamster and mouse COVID-19 models. Thus, the immunostimulatory motifs identified can be harnessed as broad-spectrum antivirals, but should be avoided, for example, when designing siRNAs.

Recognition of duplex RNAs by cellular RNA sensors plays a central role in host response to infections by initiating signaling cascades that induce secretion of interferon (IFN) and subsequent upregulation of hundreds of interferon-stimulated genes (ISGs). This pathway therefore also serves as a potent point of therapeutic intervention in a broad range of viral diseases. Duplex RNAs with various structural features have been identified that are recognized by the three cellular RNA sensors that are responsible for this innate immune response (Schlee and Hartmann, 2016). One of these, toll-like receptor 3 (TLR3), is located on the cell membrane and the endosomal membrane, while the other two-retinoic acid inducible gene I (RIG-I) and melanoma differentiation associated gene 5 (MDA5)—are located in the cytosol. Long forms of duplex RNA are recognized by these sensors based on their length (i.e., independently of the structure of their 5′ ends) with TLR3 recognizing duplex RNAs >35 bp and MDA5 sensing duplex RNAs >300 bp (Kato et al., 2008). Past reports have revealed that a short stretch of duplex RNA (>19 bp) can be recognized by RIG-I, but only if a triphosphate or a diphosphate is present at its 5′ end and if the end is blunt with no overhangs (Jiang et al., 2011; Ren et al., 2019a, b; Schlee and Hartmann, 2016).

Duplex RNA-mediated innate immune stimulation is a two-edged sword. For example, in the case of respiratory infections, such as those caused by pandemic viruses (e.g., SARS-CoV-2, SARS-CoV, MERS-CoV, influenza, etc.), RNA-mediated activation of this innate immune response provides the first line of host defense against the invading pathogen. However, on the other hand, the use of duplex RNAs for RNA interference (RNAi) approaches can result in undesired immunological off-target effects and misinterpretation of experimental results (Homung et al., 2005; Kim et al., 2004; Marques and Williams, 2005; Meng and Lu, 2017; Robbins et al., 2008; Setten et al., 2019; Sledz et al., 2003). Thus, gaining greater insight into the mechanism by which cells sense and respond to duplex RNAs could have broad impact in biology and medicine.

Data presented herein show the discovery a class of new immunostimulatory RNAs via the use of >200 small interfering RNAs (siRNAs) to identify influenza infection-associated host genes in human lung epithelial cells. These short duplex RNAs potently induce type I and type III interferons (IFN-I/III), in a wide type of cells but lack any sequence or structure characteristics of known immunostimulatory RNAs. Systematic mechanistic analysis revealed that these immunostimulatory RNAs specifically activate the RIG-I/IRF3 pathway by binding directly to RIG-I, and that this only occurs when these short RNAs have a conserved overhanging sequence motif (sense strand: 5′-C, antisense strand: 3′-GGG) and a minimum length of 20 bases. The conserved overhanging motif is responsible for the formation of RNA dimers through Hoogsteen base pairing. Interestingly, these immunostimulatory RNAs are capable of inducing IFN production regardless of whether they have blunt or overhanging ends, terminal hydroxyl or mono-phosphate groups, RNA base- or DNA base-ends, in contrast to previously described immunostimulatory RNAs that require 5′-di or -triphosphates to activate cellular RNA sensors (Ren et al., 2019a, b). The RNA-mediated IFN-I/III production resulted in significant inhibition of infections by multiple human respiratory viruses, including influenza viruses and SARS-CoV-2 in established cell lines, human Lung Airway and Alveolus Chips that have been previously shown to recapitulate human lung pathophysiology (Benam et al., 2016; Huh et al., 2010; Si et al., 2021), and in a mouse COVID-19 model. Data provided herein indicate that development of siRNAs that avoid undesired immune activation should avoid inclusion of the “GGG,” and paves the way for the development of a new class of RNA therapeutics for the prevention and treatment of respiratory virus infections.

### Results

### Discovery of IFN-I Pathway-Activating Immunostimulatory RNAs

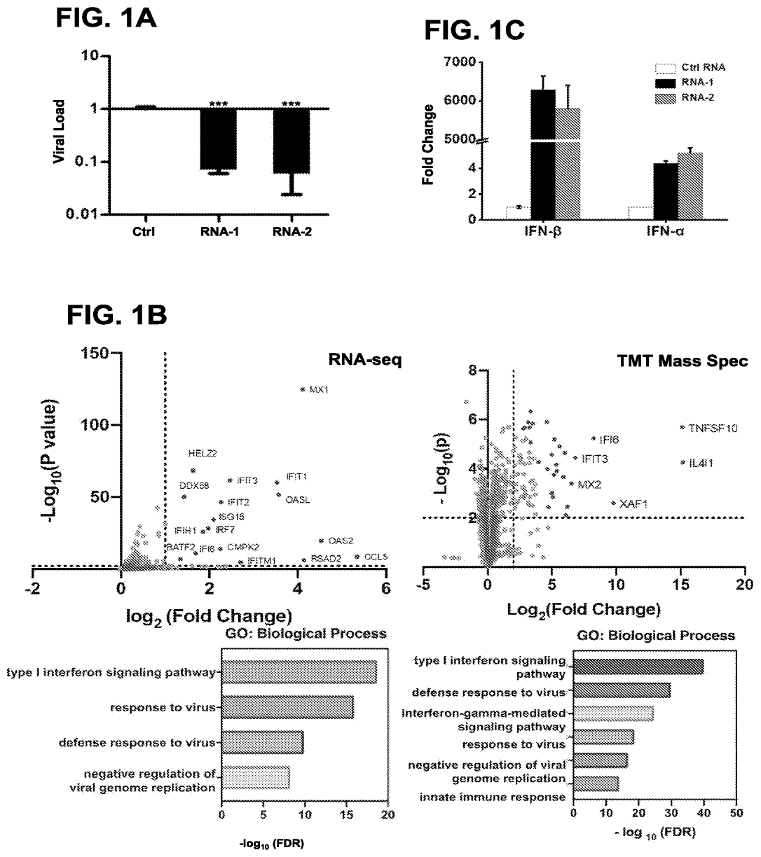

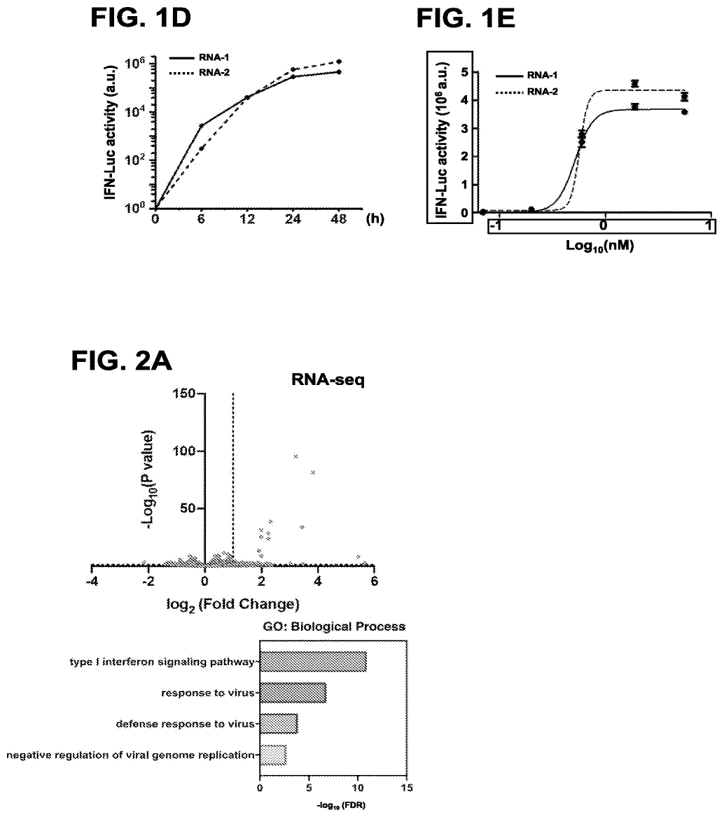

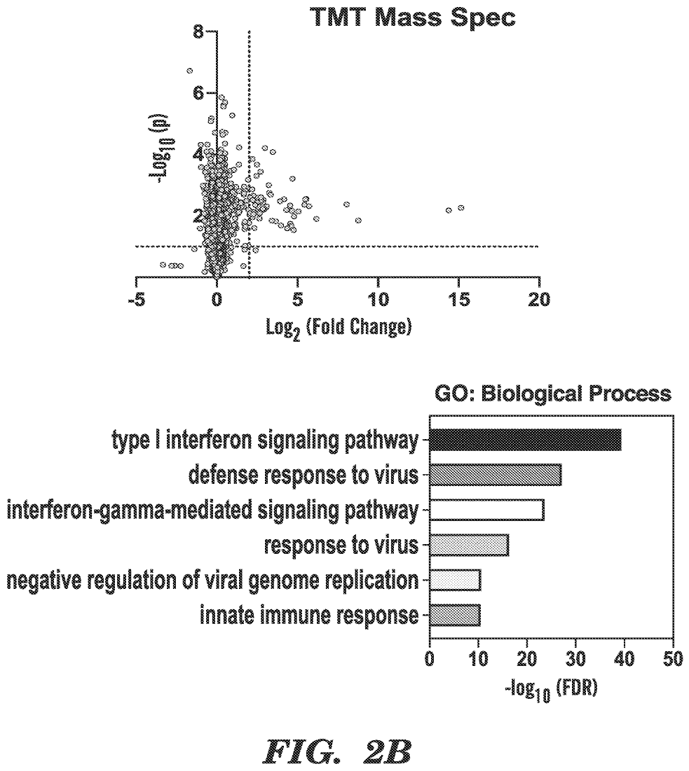

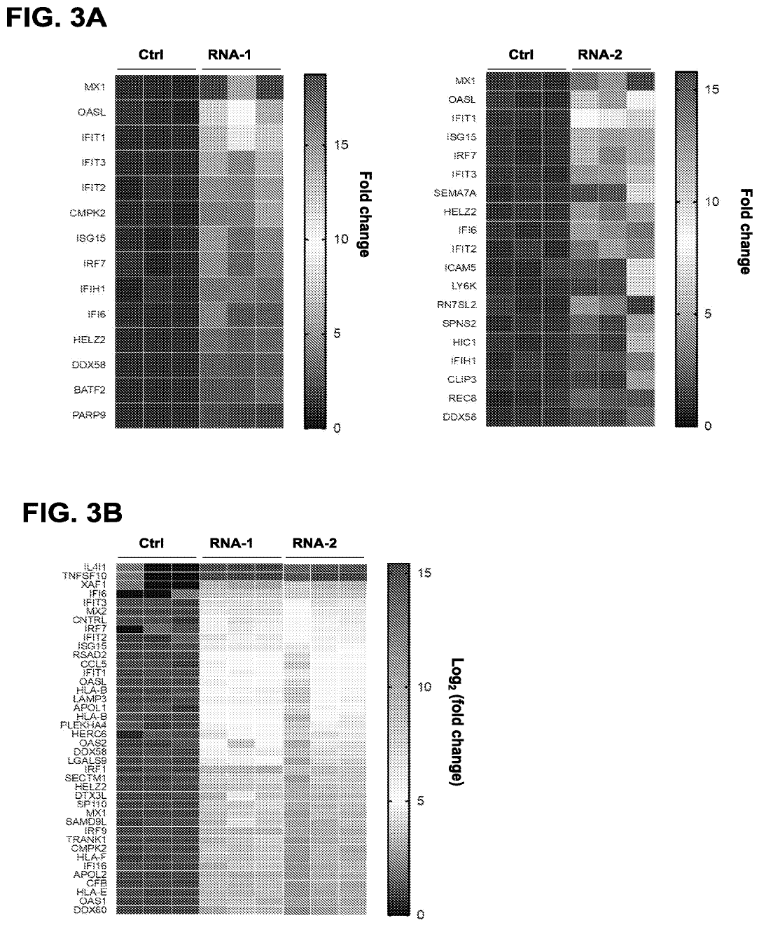

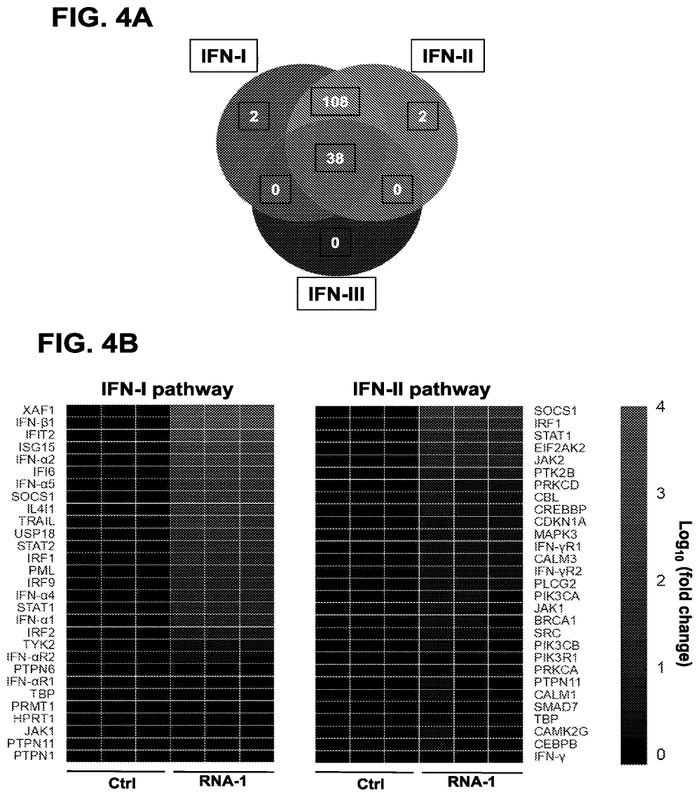

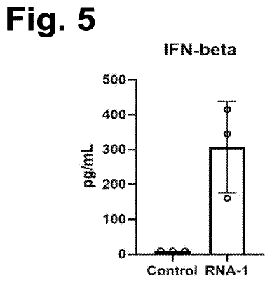

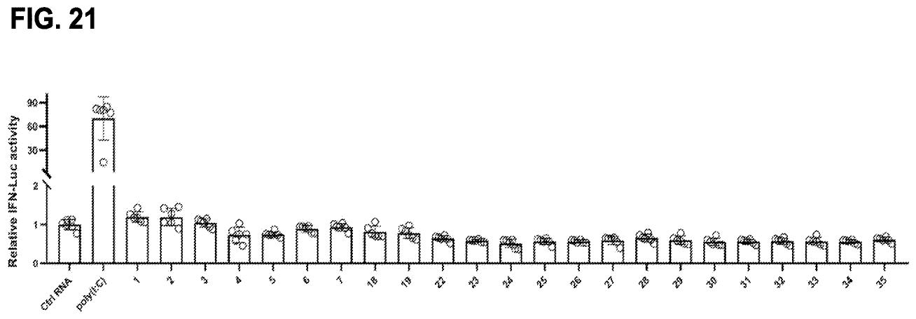

While using >200 siRNAs to identify host genes that mediate human A549 lung epithelial cell responses to influenza A/WSN/33 (HIN1) infection, it was found that transfection of two siRNAs (RNA-1 and RNA-2) inhibited H1N1 replication by more than 90% (FIG. 1A). To explore the mechanism of action of these siRNAs, the transcriptome and proteome of A549 cells transfected with RNA-1 (FIG. 1B) and RNA-2 (FIGS. 2A and 2B) were profiled, which respectively target the long non-coding RNAs (lncRNAs) DGCR5 and LINC00261, and a scrambled siRNA was used as a control. RNA-seq analysis showed that RNA-1 upregulates the expression of 21 genes by more than 2-fold (p value threshold of 0.01) (FIG. 1B left and FIG. 3A). Gene Oncology (GO) enrichment analysis revealed that these genes are involved in IFN-I signaling pathway and host defense response to viral infections (FIG. 1B left), including MX1, OASL, IFIT1, and ISG15 (FIG. 3A left). In parallel, Tandem Mass Tag Mass Spectrometry (TMT Mass Spec) quantification demonstrated upregulation of 73 proteins by more than 4-fold (p value threshold of 0.01), including IL4I1, TNFSF10, XAF1, IFI6, and IFIT3 (FIG. 1B right and FIG. 3B). GO enrichment analysis of these upregulated proteins also confirmed an association between treatment of RNA-1 and induction of the IFN-I pathway (FIG. 1B right and FIG. 4A). Quantitative reverse transcription polymerase chain reaction (qRT-PCR) assay independently validated that RNA-1 preferentially activates the IFN-I pathway relative to the Type II or III IFN pathways (FIG. 4B), with IFN-β being induced to much higher levels (>1,000-fold) compared to IFN-α (FIG. 1C). This potent induction of IFN-β by RNA-1 was verified at the protein level using enzyme-linked immunosorbent assay (ELISA) (FIGS. 5 and 20), and similar patterns of gene and protein expression were also observed for RNA-2 (FIG. 1C and FIGS. 2 to 3).

Further studies were carried out with additional siRNAs to further validate the function of the lncRNAs they target and it was found that knockdown of DGCR5 or LINC00261 by these other siRNAs did not induce IFN production. This was surprising because since the inception of RNA interference technology, short duplex (double stranded) siRNAs have been known to induce IFN-I (Kim et al., 2004; Sledz et al., 2003) and thus subsequent design of these molecules, including the ones used in data presented herein, were optimized to avoid this action and potential immunomodulatory side effects (Kim et al., 2005). siRNAs synthesized by phage polymerase that have a 5′-triphosphate end can trigger potent induction of IFN-α and -3 (Kim et al., 2004), and siRNAs containing 9 nucleotides (5′-GUCCUUCAA-3′) at the 3′ end can induce IFN-α through TLR-7 (Homung et al., 2005). Notably, RNAs with a 5′-diphosphate end can induce IFN-I as well (Goubau et al., 2014), but the synthetic duplex RNAs used herein do not have any of these sequence or structural properties. Thus, the data presented herein indicated that the two specific RNAs were found to be potent IFN-I inducers (RNA-1 and RNA-2) may represent new immunostimulatory RNAs.

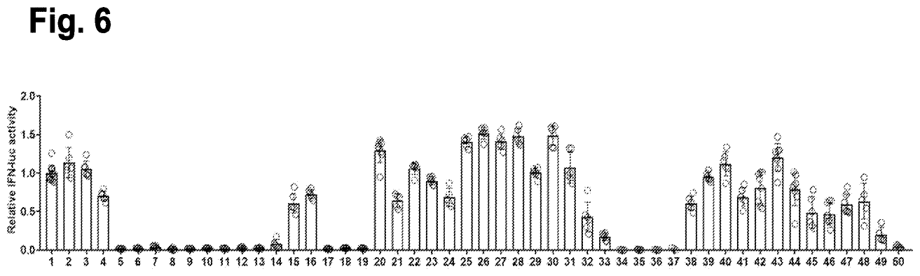

To explore this further, IFN-I production induced by the two putative immunostimulatory RNAs were assessed using an A549-Dual™ IFN reporter cell line, which stably expresses luciferase genes driven by promoters containing IFN-stimulated response elements (Tissari et al., 2005). These studies revealed that both RNA-1 and -2 induce IFN production beginning as early as 6 hours post transfection, consistent with IFN-I being an early-response gene in innate immunity, and high levels of IFN expression were sustained for at least 24 to 48 hours (FIG. 1D). A dose-dependent induction of IFN production by these duplex RNAs was also observed over the nM range (FIG. 1E). In addition, similar effects were observed when RNA-3 was tested, which was originally designed as a siRNA to knockdown another lncRNA, LINC00885 (FIG. 6, Table 1). Notably, all three immunostimulatory dsRNAs that specifically upregulate strong IFN-I responses with high efficiency share a common motif (sense strand: 5′-C, antisense strand: 3′-GGG).

### These Short Duplex RNAs Bind Directly to RIG-I

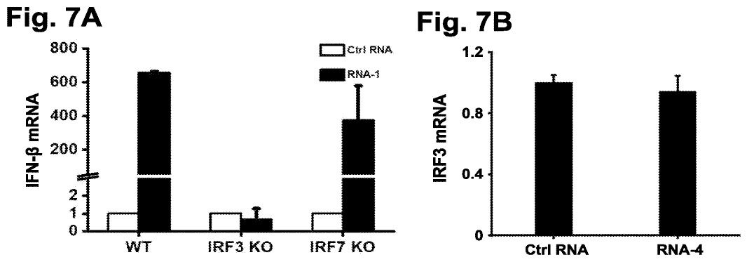

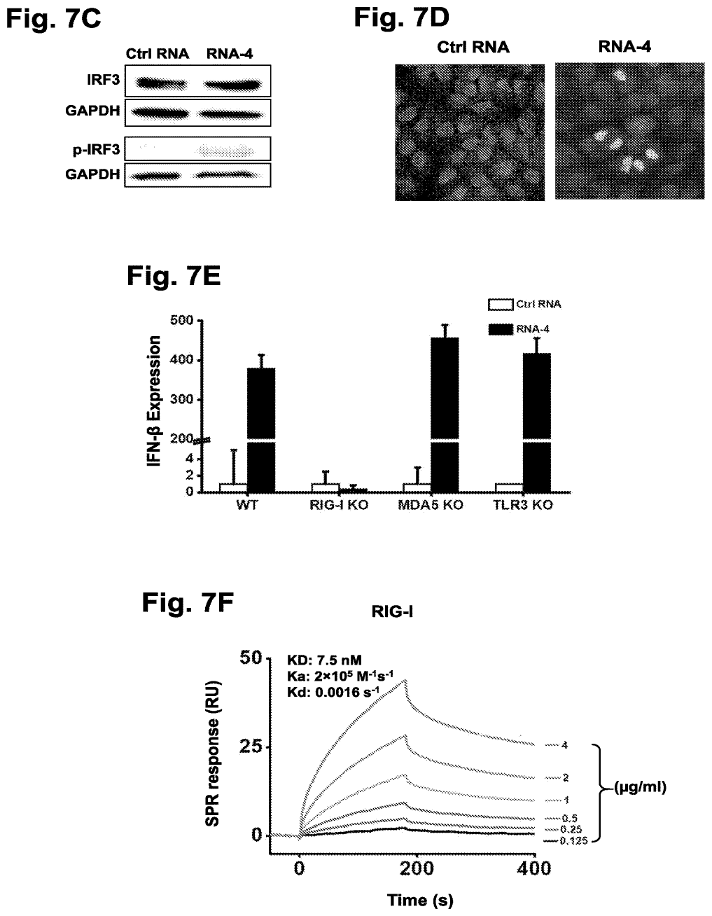

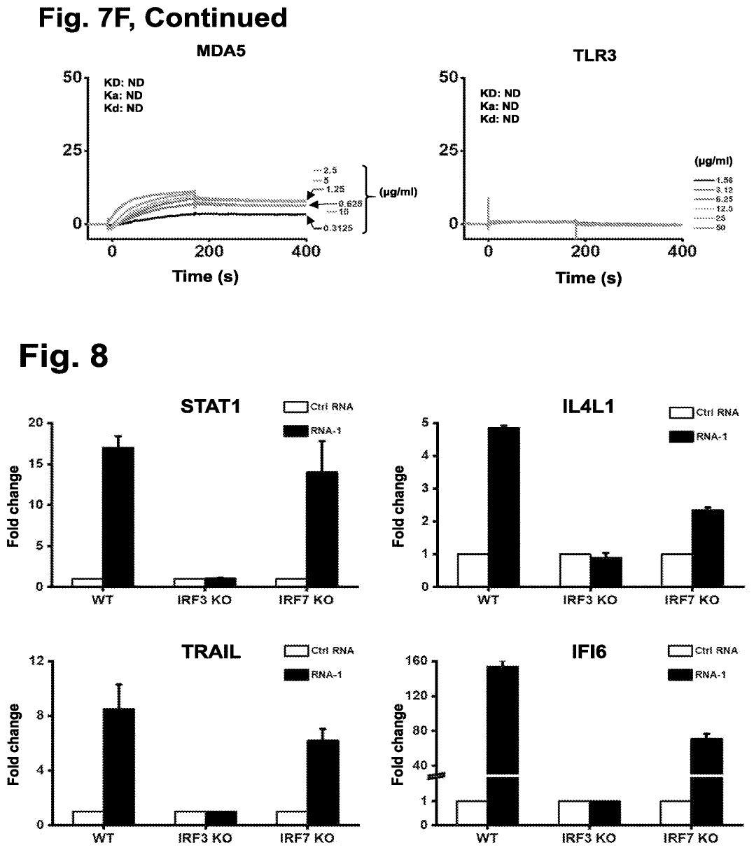

Transcription factor interferon regulatory factor 3 (IRF3) and 7 (IRF7) play vital roles in IFN-I production (Liu et al., 2015; Wang et al., 2017). Using IRF3 knockout (KO) and IRF7 KO cells, it was found that loss of IRF3, but not IRF7, completely abolished the ability of RNA-1 to induce IFN-β (FIG. 7A) and downstream ISGs, including STAT1, IL4L1, TRAIL, and IF16 (FIG. 8). IRF3 is the master and primary transcriptional activator of IFN-I and its induction of IFN-I involves a cascade of events, including IRF3 phosphorylation, dimerization, and nuclear translocation (Fitzgerald et al., 2003; Zhou et al., 2019). To alleviate potential interference from host gene knockdown by RNA-1 that was developed as an siRNA, further mechanistic studies were performed using RNA-4, which contains the common motif of RNA-1, -2, and -3 that were hypothesized and proved to be involved in the immunostimulatory activity, but does not target (silence) any host genes due to its remaining nucleotides were replaced by a random sequence (FIG. 6, Table 1). Although RNA-4 had no effect on IRF3 mRNA or total protein levels (FIG. 7B, 7C), it increased IRF3 phosphorylation (FIG. 7C), which is essential for its transcriptional activity (Liu et al., 2015) and subsequent translocation to the nucleus (FIG. 7D), where IRF3 acts as transcription factor that induces IFN-I expression (Fitzgerald et al., 2003; Zhou et al., 2019).

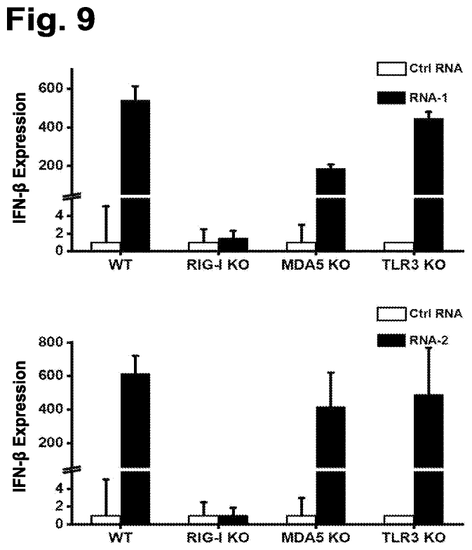

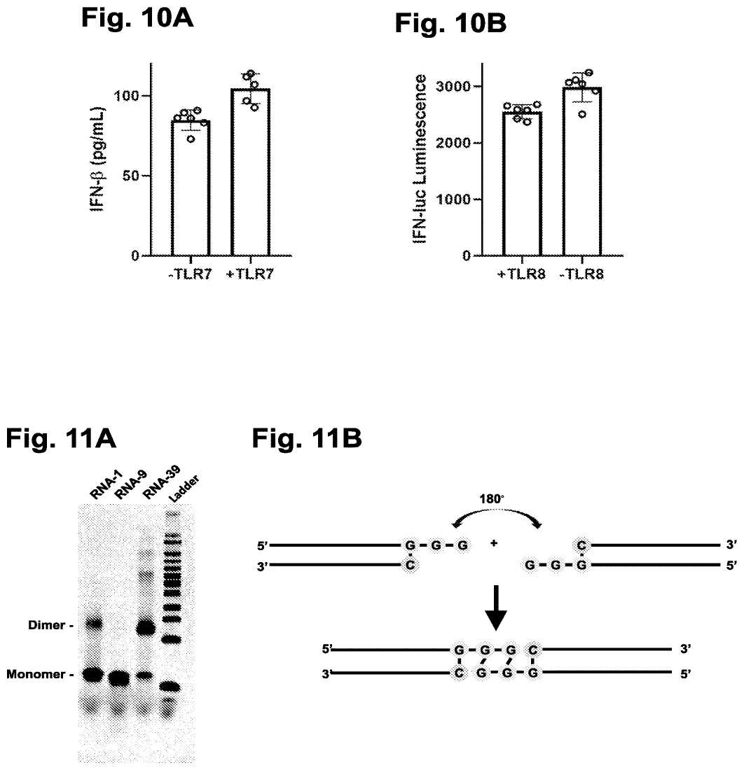

RIG-I, MDA5, and TLR3 are the main sensors upstream of IRF3 that recognize RNA (Chow et al., 2018). To investigate which of these detect the immunostimulatory short duplex RNAs, RNA-mediated production of IFN-I in RIG-I, MDA5, or TLR3 KO cells was quantified. Knockout of RIG-I completely suppressed the ability of RNA-4 (FIG. 7E) as well as RNA-1 and -2 (FIG. 9) to induce IFN-I, whereas loss of MDA5 or TLR3 had no effect on RNA-mediated IFN-I production (FIG. 7E and FIG. 9). Importantly, surface plasmon resonance (SPR) analysis revealed that RNA-1 interacts directly with the RIG-I cellular RNA sensor, rather than MDA5 or TLR3 (FIG. 7F). In addition, knockout or overexpression of other RNA sensors, such as TLR7 or TLR8, which sense RNA degradation products mediated by RNase 2 or RNase T2, did not affect the ability of these duplex RNAs to induce IFN production (FIG. 10). Thus, these short duplex RNAs stimulate IFN-I production specifically via the RIG-I/IRF3 pathway.

### Overhanging, Terminal GGG Motif Mediates IFN Activation Via Duplex RNA Dimerization

The active RNAs-1, -2, and -3 are chemically synthesized 27-mer RNA duplexes that include terminal hydroxyl groups, 2 DNA bases at the 3′ end of sense strands, and 2-base overhangs at the 3′ end of antisense strands (Table 1). Importantly, their sequence and structure features do not conform to any characteristics of existing immunostimulatory RNA molecules (Table 3), suggesting that previously unknown elements must be responsible for this immunostimulatory activity. Remarkably, even though they were designed to target different host genes, sequence alignment revealed that RNA-1, -2, and -3 contained one identical motif at their 5′ ends (sense strand: C, antisense strand: 3′-GGG-5′) (Table 1). Because all the three RNAs were potent inducers of IFN-I, it was hypothesized that this common motif may mediate their immunostimulatory activities.

To test this hypothesis, IFN-I production induced by different sequence variants of RNA-1 (Table 1) were systematically investigated using the IFN reporter-expressing cell line. Maintaining the common motif while replacing the remaining nucleotides with a random sequence (RNA-4 or RNA-5, -6, and -7, respectively vs. RNA-1, -2, and -3) did not affect the immunostimulatory activity of the duplex RNA (FIG. 6 and Table 1). However, moving the motif from 5′ GGG end to the middle region completely abolished the RNA's immunostimulatory activity (RNA-5 vs. RNA-1) (FIG. 6 and Table 1). Furthermore, the immunostimulatory activity was completely eliminated by any changes, including deletion or substitution, at the common motif (RNA-6, -7, -8, -9, -10, -11, -12, -13, -14, -15, -16, -17 vs. RNA-1) (FIG. 6 and Table 1). These data indicate that the common terminal 5′ GGG motif is necessary for IFN-I/III induction, and that this effect is sensitive to alterations in its position and sequence.

To determine whether this shared motif mediates binding to RIG-I, the immunostimulatory activity of duplex RNAs bearing an N1-2′O-methyl group was evaluated, which has been shown to block RIG-I activation by RNA when the modification occurs at the 5-terminus (Schuberth-Wagner et al., 2015). Surprisingly, the N1-2′O-methylation of the 5′-end of sense strand or 3′-end of antisense strand (RNA-15 and -16) or both simultaneously in the same duplex RNA (RNA-17) did not block RIG-I activation by RNA-1 (FIG. 6 and Table 1). In contrast, N₁-2′O-methylation of the 5′-end of the antisense strand, but not the 3′-end of the sense strand, completely blocked RIG-I activation by RNA (RNA-18, -19, and -20 vs. RNA-1) (FIG. 6 and Table 1), indicating that RNA-1 binds to RIG-I via the 5′GGG-end of its antisense strand.

Given the critical role and high conservation of the common motif in this form of duplex RNA-mediated immunostimulation, it was also sought to determine whether this common motif could mediate the formation of higher order structure of duplex RNA via an intramolecular G-quadruplex, a secondary structure that is held together by non-canonical G-G Hoogsteen base pairing (Lyu et al., 2021). Interestingly, native gel electrophoresis revealed the formation of an RNA-1 dimer, while no dimer was detected when the GG overhang was replaced with AA bases (RNA-9 vs. RNA-1) (FIG. 11A). These data suggest that the motif (sense strand: C, antisense strand: 3′-GGG-5′) mediates formation of an end-to-end RNA-1 dimer via Hoogsteen G-G base pairing, which doubles the length of the dsRNA, thereby promoting efficient binding to RIG-I via the exposed 5′ antisense strand ends of each RNA and subsequently inducing IFN production (FIG. 11B). This possibility was verified by synthesizing RNA-1 end-to-end dimer mimics (RNA-38 and -39) that have similar lengths and sequences and also exhibited potent immunostimulatory activity (FIG. 6, Table 2).

As chemically synthesized RNAs contain terminal hydroxyl groups, it was tested if adding a monophosphate at these sites affects the IFN-inducing activity. This is important to investigate because host RNAs contain a 5′-monophosphate, which has been reported to suppress RIG-I recognition (Ren et al., 2019b). However, it was found that RNA-1 containing terminal monophosphates exhibited immunostimulatory activity to a similar level as RNA-1 containing a hydroxyl groups (RNA-21, -22, -23, -24 vs. RNA-1) (FIG. 6, Table 1), suggesting that a terminal monophosphate in these short duplex RNAs is neither required for their immunostimulatory effect, nor does it interfere with the immunostimulatory activity.

As the dsRNAs contain 2 DNA bases at the 3′ end of their sense strand, it was also tested if the types of nucleosides affect the interferon-inducing activity. Interestingly, the duplex RNAs exhibited comparable immunostimulatory activity to RNA-1 regardless of whether DNA bases or RNA bases are inserted at the 3′ end of the sense strand and/or 5′ end of the antisense strand (RNA-25, -26, -27, -28 vs. RNA-1) (FIG. 6, Table 1). This was further verified by synthesizing duplex RNA dimer mimics (RNA-40 and -41 vs. RNA-39) that contain terminal DNA or RNA bases, which exhibited similar immunostimulatory activity to RNA-39 that contains 2 DNA bases at the 3′ ends of sense and antisense strands (FIG. 6, Table 2).

It was then tested if introduction of an overhang affects the IFN-inducing activity because previous reports revealed that RIG-I can be activated by blunt duplex RNAs, and that almost any type of 5′ or 3′ overhang can prevent RIG-I binding and eliminate signaling (Ren et al., 2019a). However, it was found that the overhang did not affect the IFN-inducing activity of the duplex RNAs (RNA-29, -30, and -31 vs. RNA-1) (FIG. 6, Table 1). This was also verified in studies with duplex RNA mimics (RNA-42 and -43 vs. RNA-39) that contain terminal overhangs, which induced IFN production to a similar level as RNA-39 containing blunt ends (FIG. 6, Table2).

Finally, the effects of RNA length on IFN production were tested by gradually trimming bases from the 3′ end of RNA-1. Removal of increasing numbers of bases resulted in a gradual decrease in immunostimulatory activity (RNA-32 and -33 vs. RNA-1) with complete loss of activity when 8 bases or more were removed from the 3′ end of RNA-1 (RNA-34 and -35) (FIG. 6, Table 1). Therefore, the minimal length of this new form of immunostimulatory RNA required for IFN induction is 20 bases on the antisense strand that can result in the formation of a RNA dimer containing ˜38 bases via Hoogsteen base pairing of their 5′GG ends. This is consistent with data obtained with duplex RNA end-to-end dimer mimics (RNA-44, -45, -46, -47, -48, -49, and -50 vs. RNA-38 and -39) where the minimal length of the duplex RNA dimer required for IFN induction was found to be 36 bases (FIG. 6, Table 2). Further consistent with the proposed mechanism-of-action, RIG-I knockout also abolished the IFN-inducing ability of these RNA variants (RNA-1, -2, -3, -4, -5, -6, -7, -18, -19, -22, -23, -24, -25, -26, -27, -28, -29, -30, RNA-31, -32, -33, -34, and -35) (FIG. 21). And in a final control experiment it was found that neither the single sense strand nor the single antisense strand of RNA-1 alone is sufficient to induce IFN production (RNA-36 and -37) (FIG. 6, Table 1), indicating that the double stranded RNA dimer structure is absolutely required for its immunostimulatory activity.

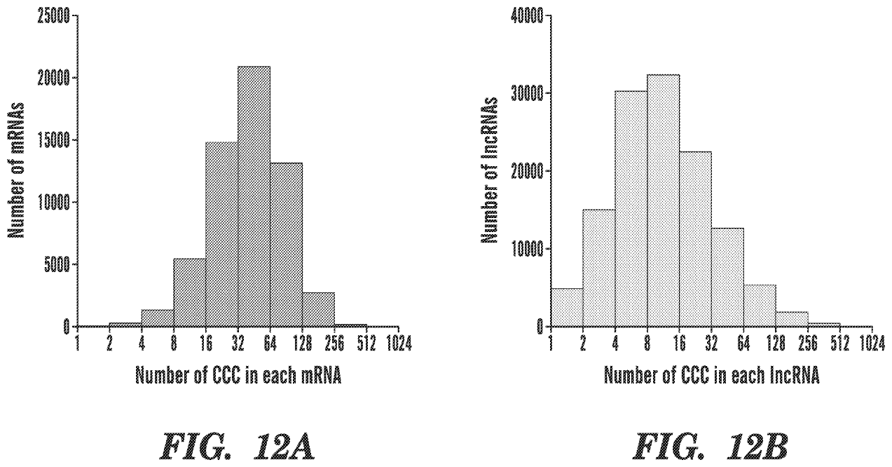

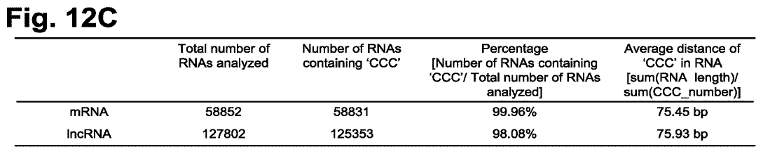

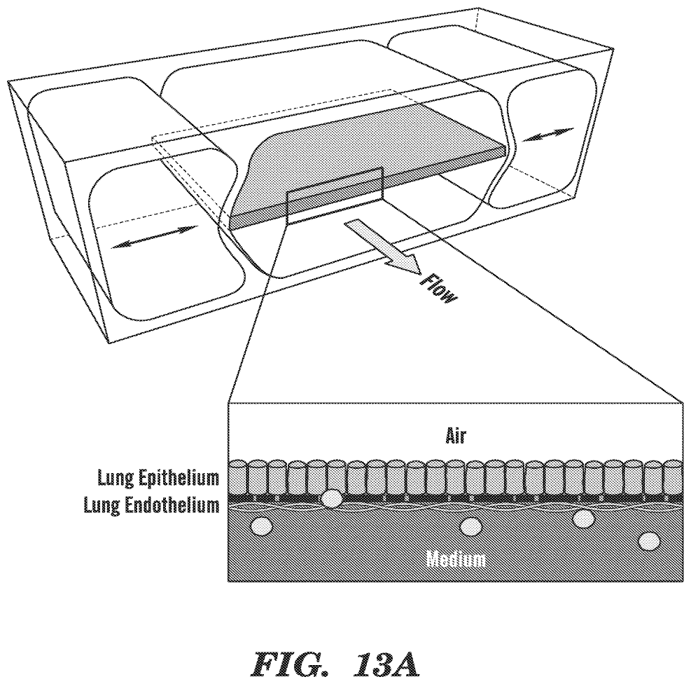

Finally, given that the overhanging motif (sense strand: C; antisense strand: 3′-GGG-5′) shown to be immunostimulatory is also found in the termini of many siRNAs, its frequency in both human mRNAs and lncRNAs was evaluated. Genome-wide sequence analysis revealed that the ‘CCC’ motif is abundant in both mRNAs and lncRNAs sequences: 99.96% of human mRNAs contain ‘CCC’ with an average distance of 75.45 bp between adjacent motifs and 98.08% of human lncRNAs contain ‘CCC’ with an average distance of 75.93 bp between adjacent motifs (FIG. 12). Thus, this indicates that the ‘GGG’ motif that mediates short duplex RNA dimerization should be avoided when an siRNA's immunostimulatory effect is undesired.

Accordingly, provided herein is a method of preparing an RNAi molecule to promote degradation of a target RNA, the method comprising: a) identifying CCC trinucleotide repeats in the sequence of a target RNA; b) selecting a 20 to nucleotide sequence in the target RNA that lacks CCC repeats as a candidate RNAi sequence; c) synthesizing an RNA molecule complementary to the sequence selected in step (b); and d) synthesizing an RNA molecule complementary to the RNA molecule synthesized in step (c), wherein combination of the RNA molecules synthesized in steps (c) and (d) produces an RNAi molecule that is less immunostimulatory than an RNAi molecule that targets the same target RNA but comprises a CCC trinucleotide repeat.

Self-Assembling dsRNAs Induce Less Proinflammatory Genes than Poly(I:C)

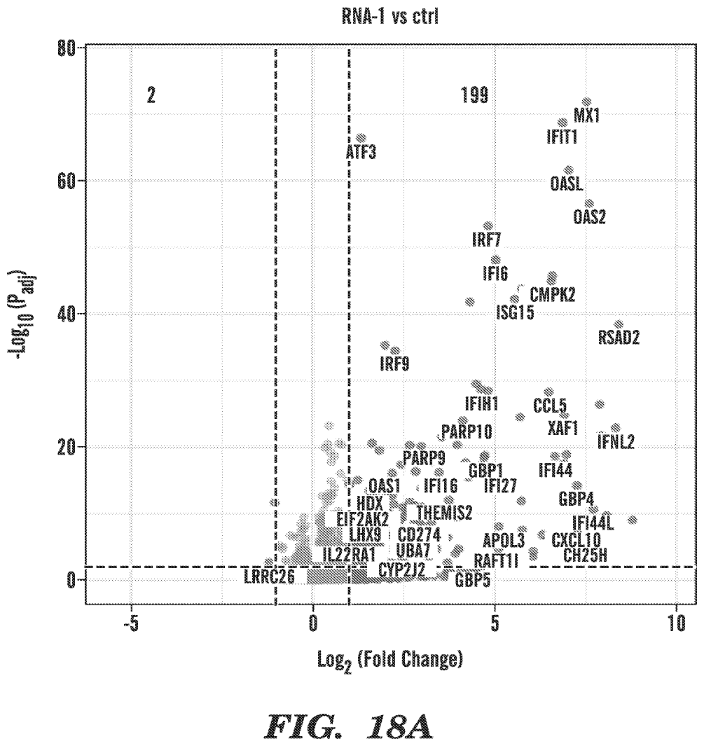

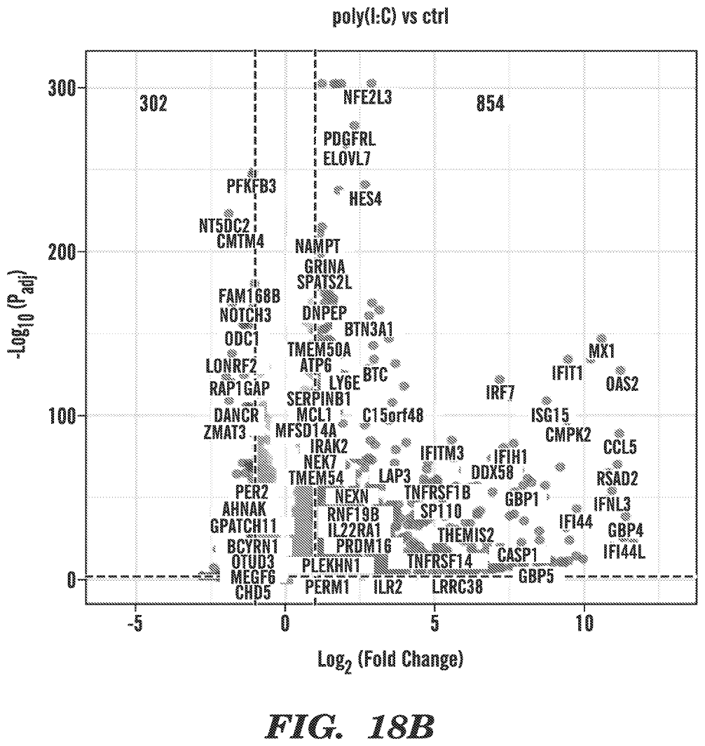

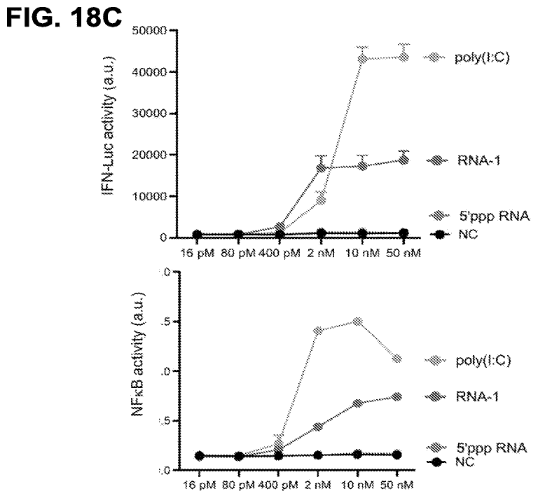

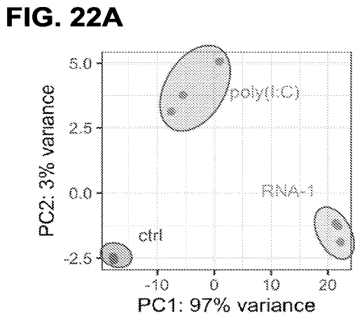

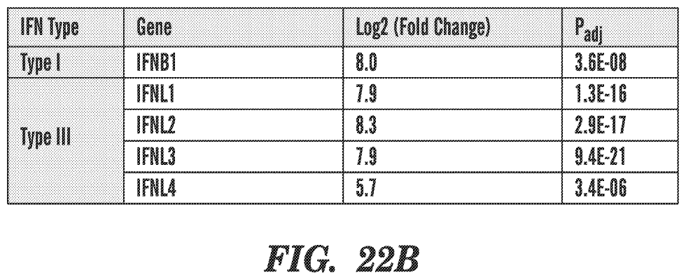

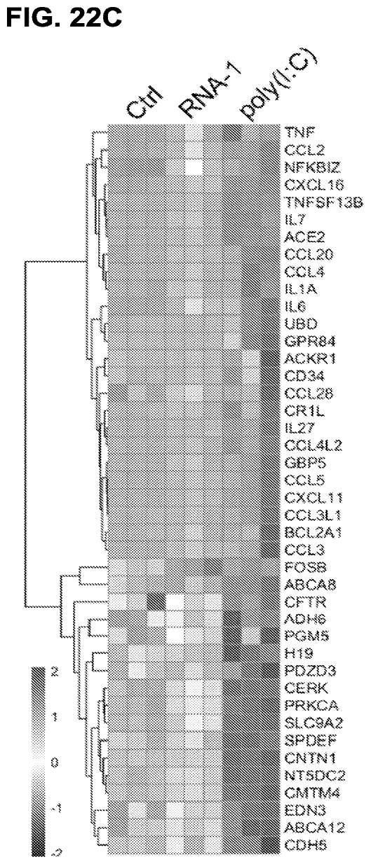

Polyinosinic:polycytidylic acid [poly(I:C)] is an immunostimulant used to simulate viral infections, which interacts with multiple pattern recognition receptors, including toll-like receptor 3 (TLR3), RIG-I, and MDA5. To compare the immunostimulatory landscape induced by RNA-1 with poly(I:C), bulk RNA-seq analysis of A549 cells transfected with the sample amounts of scrambled dsRNA as control, RNA-1, or poly(I:C) were performed for 48 hours. Principal-component analysis shows that RNA-1 and poly(I:C) induce distinct transcriptomic changes (FIG. 22A). Similar to earlier results (FIG. 1B), RNA-1 upregulated many genes that are involved in antiviral IFN response, such as MX1, OASL, IRF7, IFIT1 (FIG. 18A), as well as both Type I and Type III IFN genes (FIG. 22B). In contrast, poly(I:C) induces much broader changes in gene expression: 302 genes have decreased expression while only 2 decrease when treated with RNA-1 (FIG. 18B). A heat map also shows that many proinflammatory cytokines and chemokines, such as CXCL11, TNF, CCL2, ILIA, have much higher expression in cells transfected with poly(I:C) (FIG. 22C). In addition, a number of genes involved in ion transport and cell adhesion are decreased by poly(I:C) but not by RNA-1. Notably, many of these genes (MYO1A, NEB, ADH6, H19, ELN, etc.) were also down-regulated in SARS-CoV-2 infection. Immune responses elicited by RNA-1 and poly(I:C) were further compared. Interferon reporter assay shows that poly(I:C) induce stronger IFN responses than RNA-1 (FIG. 18C, left) despite comparable responses at the protein level (FIG. 20). But this is also accompanied by stronger induction of NF-κB activity (FIG. 18C, right), which corroborates the RNA-seq results and the observation that Poly(I:C) binds to many cellular RNA sensors in additional to RIG-I, such as Toll-like receptor 3 (TLR3) and melanoma differentiation-associated gene 5 (MDA-5), which can also induce IFN. Interestingly, 5′ppp RNA does not induce IFN under the tested concentrations (FIG. 18C), which is consistent with a recent report that this type of RNA is unable to induce strong RIG-I signaling (Linhan et al. 2018). These results indicate that, when compared to poly(I:C), dsRNAs described herein induce a more targeted antiviral response and a lower level of tissue-damaging proinflammatory responses, while having no effect on critical biological processes, such as ion transport and cell adhesion, which make them more suitable for antiviral therapeutic applications.

### Broad Spectrum Inhibition of Multiple Coronaviruses and Influenza a Viruses

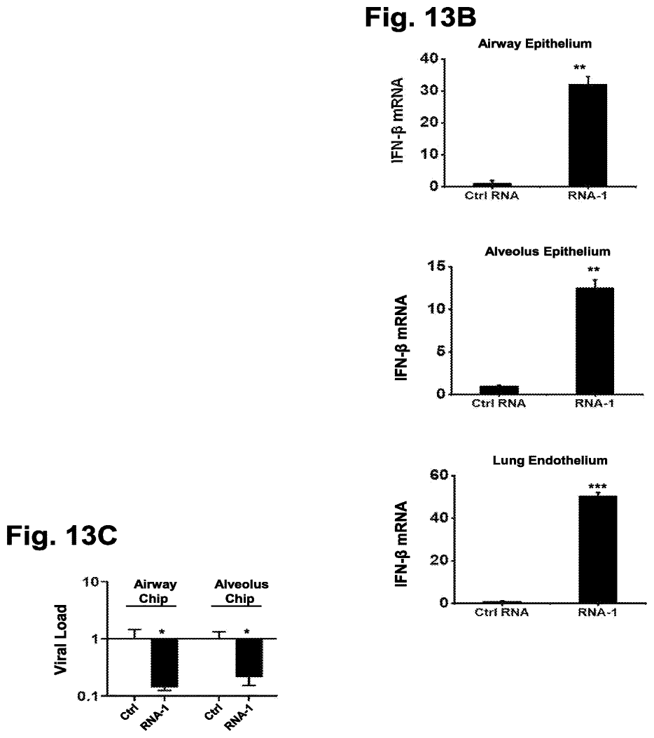

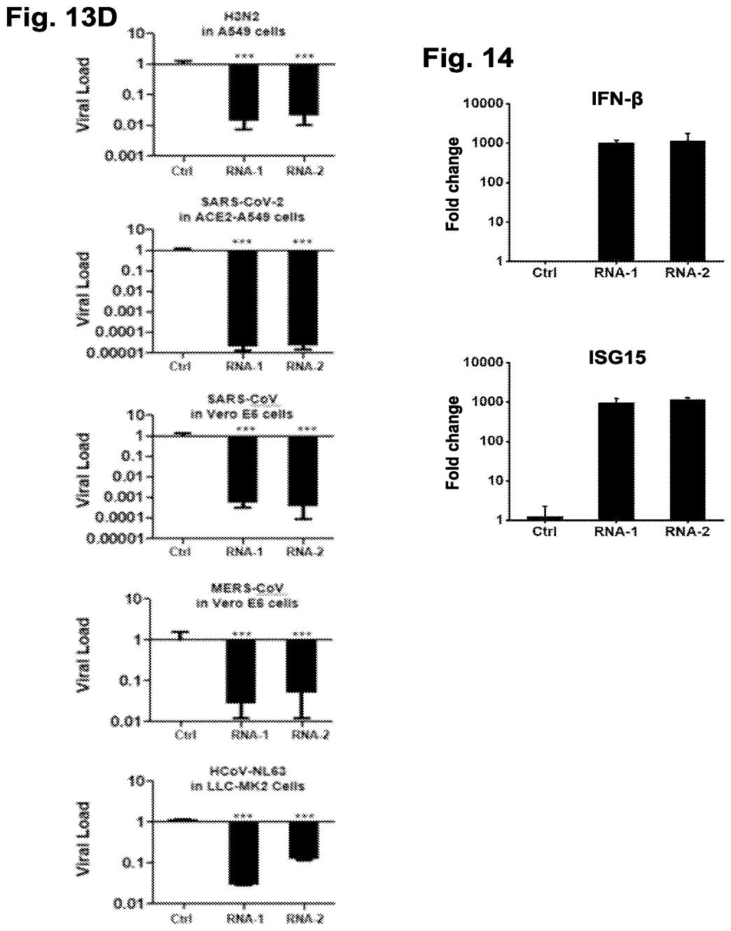

To explore the potential physiological and clinical relevance of these new RNAs that demonstrated immunostimulatory activities in established cell lines, it was investigated whether these dimers can trigger IFN-I responses in human Lung Airway and Alveolus Chip microfluidic culture devices lined by human primary lung bronchial or alveolar epithelium grown under an air-liquid interface in close apposition to a primary pulmonary microvascular endothelium cultured under dynamic fluid flow (FIG. 13A), which have been demonstrated to faithfully recapitulate human organ-level lung physiology and pathophysiology (Benam et al., 2016; Si et al., 2020; Si et al., 2019). A 12- to 30-fold increases in IFN-β expression was observed as compared to a scrambled duplex RNA control when RNA-1 was transfected into human bronchial or alveolar epithelial cells through the air channels of the human Lung Chips (FIG. 13B). In addition, treatment with RNA-1 induced robust (>40-fold) IFN-β expression in human primary lung endothelium on-chip (FIG. 13B) when it was introduced through the vascular channel.

Given the finding that RNA-1 and -2 inhibit infection by HIN1 (FIG. 1A) along with the known antiviral functions of IFN-I/III (Mesev et al., 2019), the generalizability of these effects was further explored. First, the potential of these IFN-I inducing RNAs to block infection by influenza A/HK/8/68 (H3N2) virus was examined, and then with the advent of the COVID-19 pandemic, this work was extended by carrying out similar studies with SARS-CoV-2 and related coronaviruses, SARS-CoV, MERS-CoV, and HCoV-NL63. Analysis with qPCR for viral mRNA revealed that treatment with the immunostimulatory duplex RNAs significantly suppressed infections by H3N2 influenza virus in human Lung Airway and Alveolus Chips (80-90% inhibition) and in A549 cells (>95% inhibition) (FIG. 13C, 13D), as it did with H1N1 influenza virus in A549 cells (FIG. 1A). Importantly, these same duplex RNAs inhibited MERS-CoV in Vero E6 cells and HCoV-NL63 in LLC-MK2 cells by >90% (FIG. 13D), as well as SARS-CoV in Vero E6 cells by >1,000-fold (>99.9%) (FIG. 13D). Impressively, they were even more potent inhibitors of SARS-CoV-2 infection, reducing viral load in ACE2 receptor-overexpressing A549 cells by over 10,000-fold (>99.99%) (FIG. 13D and FIG. 14), which is consistent with the observation that SARS-CoV-2 regulates IFN-I signaling differently and fails to induce its expression relative to influenza virus and other coronaviruses (Blanco-Melo et al., 2020; Galani et al., 2021).

### Immunostimulatory Duplex RNAs Inhibit SARS-CoV-2 Infection In Vivo

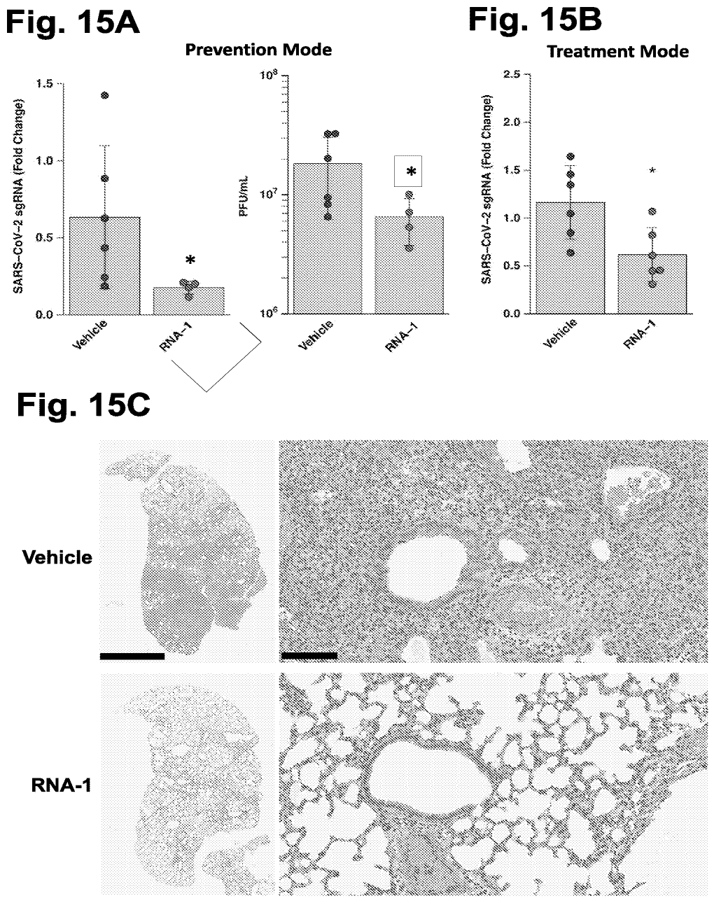

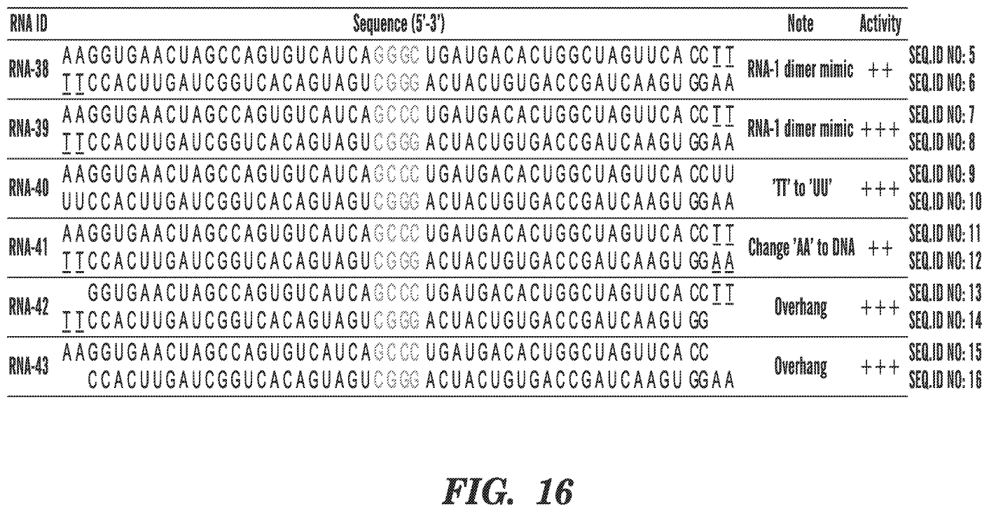

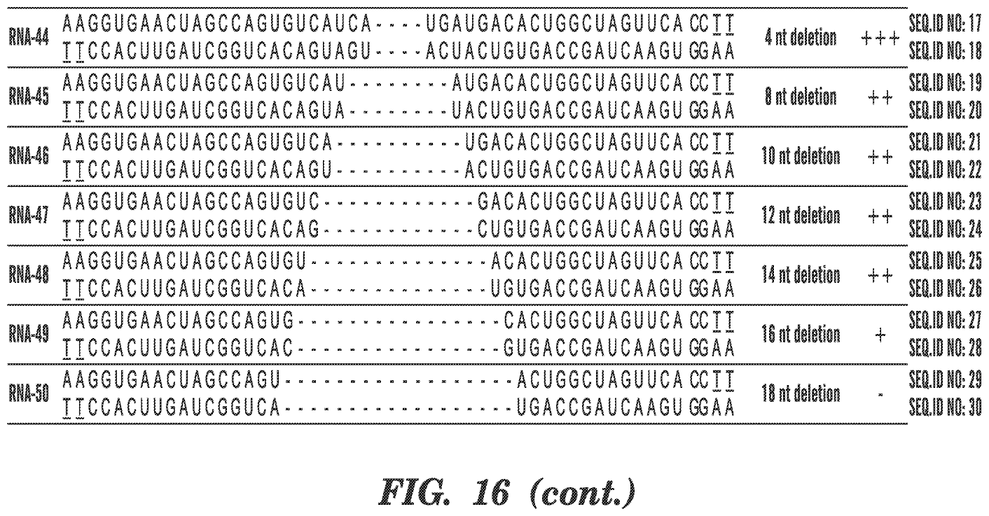

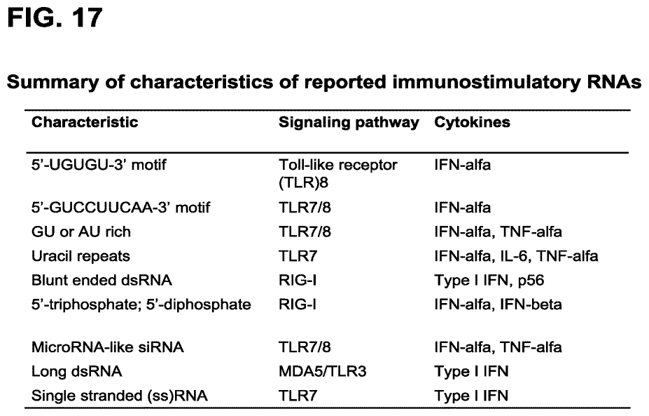

Given the potent inhibitory activity against SARS-CoV-2 observed in vitro, RNA-1 was then evaluated in a hamster COVID-19 model. RNA-1 was administered intranasally using in vivo-jetPEI® Delivery Reagent one day before the animals were infected intranasally with SARS-CoV-2 virus (10² PFU), on the day of infection, and one day post-infection. When the SARS-CoV-2 viral N transcript was measured in the lungs of these hamsters on day 2 after the viral challenge, it was found that prophylaxis with RNA-1 effectively prevented infection as it resulted in a significant (p=0.030) reduction in viral load whether measured by RT-qPCR or by quantifying viral titers using a plaque assay (p=0.032) (FIG. 15A). In addition, similar inhibition of viral infection was measured when RNA-1 was administered in a therapeutic mode by introducing it intranasally in vehicle daily for two days beginning one day after viral infection (10³ PFU) and then analyzed lungs by RT-PCR (FIG. 15B). Most importantly, histological analysis of these lungs revealed that the reduction in viral load produced by treatment with RNA-1 beginning one day after infection resulted in a major decrease in immune cell infiltration into the alveolar air spaces, which were completely obliterated and filled with cells and exudate in control infected lungs treated with vehicle alone (FIG. 15C).

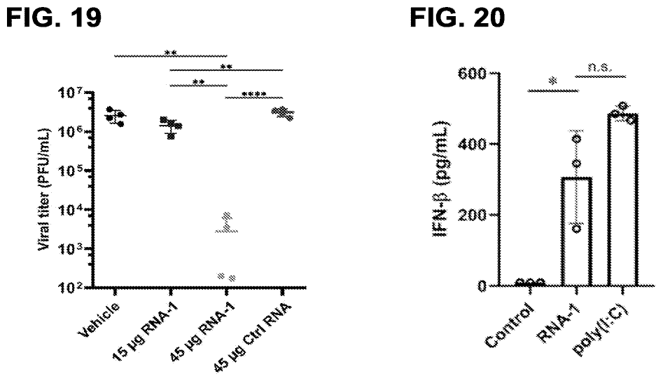

Finally, in vivo antiviral efficacy of RNA-1 against SARS-CoV-2 was assessed in a K18-hACE2 mouse model (Winkler et al. 2020). Intravenous delivery of 45 g RNA-1 using a commercial RNA delivery reagent resulted in >1,000-fold reduction of SARS-CoV-2 viral titers in the infected mouse lungs while administration of the vehicle alone or with a scrambled control RNA has no effect (FIG. 19).

### Discussion

Data presented herein show potent stimulation of IFN-I signaling, with particularly efficient induction of IFN-β relative to IFN-α, by a new class of short duplex RNAs that contain a conserved overhanging sequence motif and terminal monophosphate or hydroxyl groups in a broad spectrum of human cells. This is in contrast to previously described immunostimulatory RNAs that contain 5′-di or -triphosphates and mainly induce IFN-α or other inflammatory cytokines (Meng and Lu, 2017). Mechanistic exploration revealed that these immunostimulatory RNAs specifically activate the RIG-I/IRF3 pathway by binding directly to RIG-I, even though duplex RNAs with monophosphate groups have been previously shown to antagonize IFN signaling by RNAs with 5′-di or -triphosphates (Goubau et al., 2014; Ren et al., 2019b). By systematically investigating the effects of various sequences and lengths of these RNAs on IFN-I induction, it was identified that the immunostimulatory activity requires a minimal length of 20 bases, in addition to a conserved overhanging sequence motif (sense strand: C, antisense strand: 3′-GGG-5′). This motif mediates the formation of end-to-end duplex RNA dimers via Hoogsteen base pairing that enable its binding to RIG-I. In addition, the RNA-mediated IFN-I production that was observed resulted in significant inhibition of infections by multiple human respiratory viruses, including HIN1 and H3N2 influenza viruses, as well as coronaviruses SARS-CoV-2, SARS-CoV-1, MERS-CoV, and HCoV-NL63. Notably, these new immunostimulatory RNAs significantly reduced SARS-CoV-2 viral loads in cell lines, in human Lung Airway and Alveolus Chips containing primary lung epithelial and endothelial cells, and in vivo when administered either before or after infection in a hamster COVID-19 model. These findings raise the possibility that these IFN-I-inducing immunostimulatory RNAs could offer alternative prophylactic and therapeutic strategies for the current COVID-19 pandemic, in addition to providing potential broad-spectrum protection against a wide range of respiratory or other viruses that might emerge in the future. In particular, this new duplex RNA approach provides a clear advantage over the commonly used PRR agonist Poly(I:C), as it is fully chemically defined, easier to synthesize, and exerts a more targeted antiviral effect with less proinflammatory activity.

While much has been learned about the molecular features of viral RNAs that drive RIG-I activation, considerably less is known about the conditions under which host-derived RNAs or other exogenous RNAs activate host innate immunity in the absence of infection, as well as the mechanistic basis for this activation. Indeed, the minimally required and exclusionary features of dsRNA for RIG-I activation have proven to be complex and sometimes contradictory. Some studies suggest that 5′ diphosphate group is the minimum feature required for RIG-I binding and activation while 5′ monophosphate or 5′ hydroxyl group can antagonize the process (Ren et al. 2019a, Ren et al. 2019b; Rehwinkel et al. 2016; Ramanathan et al. 2016). However, other studies have shown that RIG-I can interact with dsRNA with 5′ monophosphate or hydroxyl group and induce an innate immune response to a certain degree (Kato et al. 2008; Hausmann et al. 2008; Brisse et al. 2019). A possible explanation for the discrepancy between these studies is that higher-order RNA structures may compensate for less-than-optimal ends (Brisse et al. 2019).

Interestingly, the conserved overhanging motif identified that contains 5′-C and 3′-GGG ends on the sense and antisense strands, respectively, appears to mediate ‘end-to-end’ dimerization of the duplex RNAs via formation of an intramolecular G-quadruplex generated by the GG overhang as any changes to this motif led to complete loss of immunostimulatory activity. The remaining exposed 5′ ends of the resultant longer dimers, in turn, appears to be responsible for binding directly to RIG-I, which thereby triggers IFN production. Consistent with this hypothesis, N1-2′O-methylation at the 5′ end of antisense strand, but not the other ends of the original short dsRNA led to complete loss of the immunostimulatory activity. All of these findings are consistent with previous research demonstrating that RIG-I recognizes the 5′ ends of longer duplex RNAs (Ren et al., 2019b). Notably, similar Hoogsteen-like pairing has been identified between trans U-U base pairs in 5′-UU overhang dsRNA fragments (Wahl et al. 2019); however, work described herein establishes for the first time that Hoogsteen base pairing can lead to generation of duplex RNAs that are highly effective RIG-I agonists. While chemical modification is not required, N1-2′O-methylation should be avoided.

Findings presented herein also led to the identification of a new form of cellular recognition of RNAs by cytoplasmic RNA sensors. At least four signaling pathways have been found to recognize immunostimulatory RNA molecules and induce the production of IFN-I and pro-inflammatory cytokines, including RIG-I, MDA5, TLR3, and TLR7/8 (Table 3). MDA5 recognizes long RNA molecules (˜0.5-7 kb in length) (Peisley et al., 2012); TLR3 detects duplex RNA molecules in the endosome that are at least 40-50 bp in length (Liu et al., 2008); TLR7 and TLR8 detect GU-rich short single strand RNAs as well as small human-made molecules, such as nucleoside analogs and imidazoquinolines (Takeuchi and Akira, 2010). RIG-I is a central component of the mammalian innate immune system, which detects pathogen-associated RNA molecules and induces rapid antiviral immune responses. Previous studies indicated that RIG-I recognizes long dsRNAs (300-1,000 bp in length), RNase L-generated small self-RNAs, or short, blunt, duplex RNAs with a 5′-di- or tri-phosphate (Homung et al., 2006; Kohlway et al., 2013; Meng and Lu, 2017; Pichlmair et al., 2006; Ren et al., 2019a; Schlee et al., 2009; Schmidt et al., 2009; Zheng et al., 2015). As described above, RIG-I is thought to be antagonized by RNAs containing 5′-monophosphate (Ren et al., 2019b), and a separate study showed that almost any type of 5′ or 3′ overhang can prevent RIG-I binding and eliminate signaling (Ren et al., 2019a). In contrast, it was observed herein that terminal hydroxyl or monophosphate groups, blunt or overhanging end, and the presence of terminal RNA or DNA bases did not affect the ability of the immunostimulatory RNAs described herein to induce IFN production, which represents a new form of RNA recognition by RIG-I. Taken together, these findings also indicate that the short duplex RNAs and their end-to-end dimers described herein represent a new class of immunostimulatory RNAs.

siRNA has become a common laboratory tool for gene silencing in biomedical research for almost two decades and a class of drugs that has recently been approved in clinics (Meng and Lu, 2017; Setten et al., 2019). However, the activation of innate immune responses by siRNAs is challenging their uses in both settings (Bartoszewski and Sikorski, 2019; Meng and Lu, 2017; Setten et al., 2019). A number of features that may elicit immune responses by siRNA have been identified (Table 3), for example, the presence of 5′ triphosphate in siRNA synthesized by phage polymerase (Kim et al., 2004) or specific sequence motifs in the sense strand of siRNA (Hornung et al., 2005). However, these features do not cover all possible scenarios, including the new immunostimulatory RNAs identified in this study. Future design of siRNA for RNAi should avoid the motif (sense strand: 5′-C; antisense strand: 3′-GGG) identified in this study to alleviate unwanted activation of innate immune responses.

While immune stimulation by siRNAs is undesired in some gene silencing applications, it can be beneficial in others, such as treatment of viral infections or cancer. The IFN response constitutes the major first line of defense against viruses, and these infectious pathogens, including SARS-CoV-2, have evolved various strategies to suppress this response (Blanco-Melo et al., 2020; Hadjadj et al., 2020). In particular, transcriptomic analyses in both human cultured cells infected with SARS-CoV-2 and COVID-19 patients revealed that SARS-CoV-2 infection produces a unique inflammatory response with very low IFN-I, IFN-III, and associated ISG responses, while still stimulating chemokine and pro-inflammatory cytokine production (Blanco-Melo et al., 2020; Hadjadj et al., 2020), and this imbalance likely contributes to the increased morbidity and mortality seen in late stage COVID-19 patients. Type I and type III IFN proteins are therefore being evaluated for their efficacy as therapeutics in preclinical models and clinical trials (Broggi et al., 2020; Park and Iwasaki, 2020; Wadman, 2020a, b). Pretreatment with IFN proteins has been shown to reduce viral titers, suggesting that induction of IFN-I responses may represent a potentially effective approach for prophylaxis or early treatment of SARS-CoV-2 infections (Lokugamage, 2020; Mantlo et al., 2020). Triple combination of IFN-31b, lopinavir, ritonavir, and ribavirin also has been recently reported to shorten the duration of viral shedding and hospital stay in patients with mild to moderate COVID-19 (Hung et al., 2020).

Consistent with these observations, results presented herein showed that pretreatment with immunostimulatory RNAs described herein resulted in a dramatic decrease in infection by SARS-CoV-2, as well as SARS-CoV, MERS-CoV, HCoV-NL63 (common cold virus) and H1N1 and H3N2 influenza viruses. Importantly, the immunostimulatory RNAs specifically activate RIG-I/IFN-I pathway but are not recognized by other cellular RNA sensors, such as TLR7, TLR8, MDA5, or TLR3. This is interesting because recent studies show that SARS-CoV-2 inhibits RIG-I signaling and clearance of infection via expression of nsp1 (Thoms et al., 2020). Importantly, the emerging SARS-CoV-2 Omicron subvariants BA.4 and BA.5 are notable for their ability to enhance innate immune suppression (Reuschl, A-K, et al. 2022). Thus, in addition to demonstrating potent antiviral effects in the COVID-19 mouse model, work described herein demonstrated that these duplex RNAs can overcome viral antagonism of human innate immunity, at least in human lung epithelial and endothelial cells maintained in Organ Chips that have been previously shown to recapitulate human lung pathophysiology with high fidelity (Jain, A., et al. 2018 and Huh, D. et al. 2012).

### REFERENCES

Bartoszewski, R., and Sikorski, A. F. (2019). Editorial focus: understanding off-target effects as the key to successful RNAi therapy. Cell Mol Biol Lett 24, 69.
Benam, K. H., Villenave, R., Lucchesi, C., Varone, A., Hubeau, C., Lee, H. H., Alves, S. E., Salmon, M., Ferrante, T. C., Weaver, J. C., et al. (2016). Small airway-on-a-chip enables analysis of human lung inflammation and drug responses in vitro. Nat Methods 13, 151-157.
Blanco-Melo, D., Nilsson-Payant, B. E., Liu, W. C., Uhl, S., Hoagland, D., Moller, R., Jordan, T X., Oishi, K., Panis, M., Sachs, D., et al. (2020). Imbalanced Host Response to SARS-CoV-2 Drives Development of COVID-19. Cell 181, 1036-1045 e1039.
Brisse, M, and Ly, H (2019). Comparative Structure and Function Analysis of the RIG-I-Like Receptors: RIG-I and MDA5. Front Immunol 10: 1586.
Broggi, A., Ghosh, S., Sposito, B., Spreafico, R., Balzarini, F., Lo Cascio, A., Clementi, N., De Santis, M., Mancini, N., Granucci, F., et al. (2020). Type III interferons disrupt the lung epithelial barrier upon viral recognition. Science.
Burke, J. M., Moon, S. L., Matheny, T., and Parker, R. (2019). RNase L Reprograms Translation by Widespread mRNA Turnover Escaped by Antiviral mRNAs. Mol Cell 75, 1203-1217 e1205.
Chow, K. T., Gale, M., Jr., and Loo, Y. M. (2018). RIG-I and Other RNA Sensors in Antiviral Immunity. Annu Rev Immunol 36, 667-694.
Elias, J. E., and Gygi, S. P. (2007). Target-decoy search strategy for increased confidence in large-scale protein identifications by mass spectrometry. Nat Methods 4, 207-214.
Elias, J. E., and Gygi, S. P. (2010). Target-decoy search strategy for mass spectrometry-based proteomics. Methods Mol Biol 604, 55-71.
Fitzgerald, K. A., McWhirter, S. M., Faia, K. L., Rowe, D. C., Latz, E., Golenbock, D. T., Coyle, A. J., Liao, S. M., and Maniatis, T. (2003). IKKepsilon and TBK1 are essential components of the IRF3 signaling pathway. Nat Immunol 4, 491-496.
Galani, I. E., Rovina, N., Lampropoulou, V., Triantafyllia, V., Manioudaki, M., Pavlos, E., Koukaki, E., Fragkou, P. C., Panou, V., Rapti, V., et al. (2021). Untuned antiviral immunity in COVID-19 revealed by temporal type 1/111 interferon patterns and flu comparison. Nat Immunol 22, 32-40.
Goubau, D., Schlee, M., Deddouche, S., Pruijssers, A. J., Zillinger, T., Goldeck, M., Schuberth, C., Van der Veen, A. G., Fujimura, T., Rehwinkel, J., et al. (2014). Antiviral immunity via RIG-I-mediated recognition of RNA bearing 5′-diphosphates. Nature 514, 372-375.
Gygi, J. P., Yu, Q., Navarrete-Perea, J., Rad, R., Gygi, S. P., and Paulo, J. A. (2019). Web-Based Search Tool for Visualizing Instrument Performance Using the Triple Knockout (TKO) Proteome Standard. J Proteome Res 18, 687-693.
Hadjadj, J., Yatim, N., Barnabei, L., Corneau, A., Boussier, J., Smith, N., Pere, H., Charbit, B., Bondet, V., Chenevier-Gobeaux, C., et al. (2020). Impaired type I interferon activity and inflammatory responses in severe COVID-19 patients. Science 369, 718-724.
Hausmann, S, Marq, J B, Tapparel, C, Kolakofsky, D, and Garcin, D (2008). RIG-I and dsRNA-induced IFNbeta activation. PLoS One 3: e3965.
Hornung, V., Ellegast, J., Kim, S., Brzozka, K., Jung, A., Kato, H., Poeck, H., Akira, S., Conzelmann, K. K., Schlee, M., et al. (2006). 5′-Triphosphate RNA is the ligand for RIG-I. Science 314, 994-997.
Hornung, V., Guenthner-Biller, M., Bourquin, C., Ablasser, A., Schlee, M., Uematsu, S., Noronha, A., Manoharan, M., Akira, S., de Fougerolles, A., et al. (2005). Sequence-specific potent induction of IFN-alpha by short interfering RNA in plasmacytoid dendritic cells through TLR7. Nat Med 11, 263-270.
Huang da, W., Sherman, B. T., and Lempicki, R. A. (2009). Systematic and integrative analysis of large gene lists using DAVID bioinformatics resources. Nat Protoc 4, 44-57.
Huh, D., Leslie, D. C., Matthews, B. D., Fraser, J. P., Jurek, S., Hamilton, G. A., Thorneloe, K. S., McAlexander, M. A., and Ingber, D. E. (2012). A human disease model of drug toxicity-induced pulmonary edema in a lung-on-a-chip microdevice. Sci Transl Med 4, 159ra147.
Huh, D., Matthews, B. D., Mammoto, A., Montoya-Zavala, M., Hsin, H. Y., and Ingber, D. E. (2010). Reconstituting organ-level lung functions on a chip. Science 328, 1662-1668.
Huh, D, Leslie, D C, Matthews, B D, Fraser, J P, Jurek, S, Hamilton, G A, et al. (2012). A human disease model of drug toxicity-induced pulmonary edema in a lung-on-a-chip microdevice. Sci Transl Med 4: 159ra147.
Hung, I. F., Lung, K. C., Tso, E. Y., Liu, R., Chung, T. W., Chu, M. Y., Ng, Y. Y., Lo, J., Chan, J., Tam, A. R., et al. (2020). Triple combination of interferon beta-1b, lopinavir-ritonavir, and ribavirin in the treatment of patients admitted to hospital with COVID-19: an open-label, randomised, phase 2 trial. Lancet 395, 1695-1704.
Huttlin, E. L., Jedrychowski, M. P., Elias, J. E., Goswami, T., Rad, R., Beausoleil, S. A., Villen, J., Haas, W., Sowa, M. E., and Gygi, S. P. (2010). A tissue-specific atlas of mouse protein phosphorylation and expression. Cell 143, 1174-1189.
Jain, A., Barrile, R., van der Meer, A. D., Mammoto, A., Mammoto, T., De Ceunynck, K., Aisiku, O., Otieno, M. A., Louden, C. S., Hamilton, G. A., et al. (2018). Primary Human Lung Alveolus-on-a-chip Model of Intravascular Thrombosis for Assessment of Therapeutics. Clin Pharmacol Ther 103, 332-340.
Jiang, F., Ramanathan, A., Miller, M. T., Tang, G. Q., Gale, M., Jr., Patel, S. S., and Marcotrigiano, J. (2011). Structural basis of RNA recognition and activation by innate immune receptor RIG-I. Nature 479, 423-427.
Kato, H., Takeuchi, O., Mikamo-Satoh, E., Hirai, R., Kawai, T., Matsushita, K., Hiiragi, A., Dermody, T. S., Fujita, T., and Akira, S. (2008). Length-dependent recognition of double-stranded ribonucleic acids by retinoic acid-inducible gene-I and melanoma differentiation-associated gene 5. J Exp Med 205, 1601-1610.
Kim, D. H., Behlke, M. A., Rose, S. D., Chang, M. S., Choi, S., and Rossi, J. J. (2005). Synthetic dsRNA Dicer substrates enhance RNAi potency and efficacy. Nat Biotechnol 23, 222-226. Kim, D. H., Longo, M., Han, Y., Lundberg, P., Cantin, E., and Rossi, J. J. (2004). Interferon induction by siRNAs and ssRNAs synthesized by phage polymerase. Nat Biotechnol 22, 321-325.
Kohlway, A., Luo, D., Rawling, D. C., Ding, S. C., and Pyle, A. M. (2013). Defining the functional determinants for RNA surveillance by RIG-I. EMBO reports 14, 772-779.
Liu, L., Botos, I., Wang, Y., Leonard, J. N., Shiloach, J., Segal, D. M., and Davies, D. R. (2008). Structural basis of toll-like receptor 3 signaling with double-stranded RNA. Science 320, 379-381.
Linehan, M M, Dickey, T H, Molinari, E S, Fitzgerald, M E, Potapova, O, Iwasaki, A, et al. (2018). A minimal RNA ligand for potent RIG-I activation in living mice. Sci Adv 4: e1701854.
Liu, S., Cai, X., Wu, J., Cong, Q., Chen, X., Li, T., Du, F., Ren, J., Wu, Y. T., Grishin, N. V., et al. (2015). Phosphorylation of innate immune adaptor proteins MAVS, STING, and TRIF induces IRF3 activation. Science 347, aaa2630.
Lokugamage, K. G., Hage, A., Schindewolf, C., Rajsbaum, R., and Menachery, V. D. (2020). SARS-CoV-2 is sensitive to type I interferon pretreatment. bioRxiv.
Lyu, K., Chow, E. Y., Mou, X., Chan, T. F., and Kwok, C. K. (2021). RNA G-quadruplexes (rG4s): genomics and biological functions. Nucleic Acids Res.
Mantlo, E., Bukreyeva, N., Maruyama, J., Paessler, S., and Huang, C. (2020). Antiviral activities of type I interferons to SARS-CoV-2 infection. Antiviral Res 179, 104811.
Marques, J. T., and Williams, B. R. (2005). Activation of the mammalian immune system by siRNAs. Nat Biotechnol 23, 1399-1405.
McAlister, G. C., Huttlin, E. L., Haas, W., Ting, L., Jedrychowski, M. P., Rogers, J. C., Kuhn, K., Pike, I., Grothe, R. A., Blethrow, J. D., et al. (2012). Increasing the multiplexing capacity of TMTs using reporter ion isotopologues with isobaric masses. Anal Chem 84, 7469-7478.
Meng, Z., and Lu, M. (2017). RNA Interference-Induced Innate Immunity, Off-Target Effect, or Immune Adjuvant? Front Immunol 8, 331.
Mesev, E. V., LeDesma, R. A., and Ploss, A. (2019). Decoding type I and III interferon signalling during viral infection. Nat Microbiol 4, 914-924.
Navarrete-Perea, J., Yu, Q., Gygi, S. P., and Paulo, J. A. (2018). Streamlined Tandem Mass Tag (S L-TMT) Protocol: An Efficient Strategy for Quantitative (Phospho)proteome Profiling Using Tandem Mass Tag-Synchronous Precursor Selection-MS3. J Proteome Res 17, 2226-2236.
Park, A., and Iwasaki, A. (2020). Type I and Type III Interferons—Induction, Signaling, Evasion, and Application to Combat COVID-19. Cell Host Microbe 27, 870-878.
Paulo, J. A., O'Connell, J. D., and Gygi, S. P. (2016). A Triple Knockout (TKO) Proteomics Standard for Diagnosing Ion Interference in Isobaric Labeling Experiments. J Am Soc Mass Spectrom 27, 1620-1625.
Peisley, A., Jo, M. H., Lin, C., Wu, B., Orme-Johnson, M., Walz, T., Hohng, S., and Hur, S. (2012). Kinetic mechanism for viral dsRNA length discrimination by MDA5 filaments. Proc Natl Acad Sci USA 109, E3340-3349.
Pichlmair, A., Schulz, O., Tan, C. P., Naslund, T. I., Liljestrom, P., Weber, F., and Reis e Sousa, C. (2006). RIG-I-mediated antiviral responses to single-stranded RNA bearing 5′-phosphates. Science 314, 997-1001.
Ramanathan, A, Devarkar, S C, Jiang, F, Miller, M T, Khan, A G, Marcotrigiano, J, et al. (2016). The autoinhibitory CARD2-Hel2i Interface of RIG-I governs RNA selection. Nucleic Acids Res 44: 896-909.
Ren, X., Linehan, M. M., Iwasaki, A., and Pyle, A. M. (2019a). RIG-I Recognition of RNA Targets: The Influence of Terminal Base Pair Sequence and Overhangs on Affinity and Signaling. Cell Rep 29, 3807-3815 e3803.
Ren, X., Linehan, M. M., Iwasaki, A., and Pyle, A. M. (2019b). RIG-I Selectively Discriminates against 5′-Monophosphate RNA. Cell Rep 26, 2019-2027 e2014.
Rehwinkel, J, and Gack, M U (2020). RIG-I-like receptors: their regulation and roles in RNA sensing. Nat Rev Immunol 20: 537-551.
Reuschl, A-K, Thorne, L G, Whelan, MVX, Mesner, D, Ragazzini, R, Dowgier, G, et al. (2022). Enhanced innate immune suppression by SARS-CoV-2 Omicron subvariants BA.4 and BA.5. bioRxiv: 2022.2007.2012.499603.
Robbins, M., Judge, A., Ambegia, E., Choi, C., Yaworski, E., Palmer, L., McClintock, K., and MacLachlan, I. (2008). Misinterpreting the therapeutic effects of small interfering RNA caused by immune stimulation. Hum Gene Ther 19, 991-999.
Schlee, M., and Hartmann, G. (2016). Discriminating self from non-self in nucleic acid sensing. Nat Rev Immunol 16, 566-580.
Schlee, M., Roth, A., Hornung, V., Hagmann, C. A., Wimmenauer, V., Barchet, W., Coch, C., Janke, M., Mihailovic, A., Wardle, G., et al. (2009). Recognition of 5′ triphosphate by RIG-I helicase requires short blunt double-stranded RNA as contained in panhandle of negative-strand virus. Immunity 31, 25-34.
Schmidt, A., Schwerd, T., Hamm, W., Hellmuth, J. C., Cui, S., Wenzel, M., Hoffmann, F. S., Michallet, M. C., Besch, R., Hopfner, K. P., et al. (2009). 5′-triphosphate RNA requires base-paired structures to activate antiviral signaling via RIG-I. Proc Natl Acad Sci USA 106, 12067-12072.
Schuberth-Wagner, C., Ludwig, J., Bruder, A. K., Herzner, A. M., Zillinger, T., Goldeck, M., Schmidt, T., Schmid-Burgk, J. L., Kerber, R., Wolter, S., et al. (2015). A Conserved Histidine in the RNA Sensor RIG-I Controls Immune Tolerance to N1-2′O-Methylated Self RNA. Immunity 43, 41-51.
Setten, R. L., Rossi, J. J., and Han, S. P. (2019). The current state and future directions of RNAi-based therapeutics. Nat Rev Drug Discov 18, 421-446.
Si, L., Bai, H., Rodas, M., Cao, W., Oh, C. Y., Jiang, A., Moller, R., Hoagland, D., Oishi, K., Horiuchi, S., et al. (2021). A human-airway-on-a-chip for the rapid identification of candidate antiviral therapeutics and prophylactics. Nat Biomed Eng.
Si, L., Bai, H., Rodas, M., Cao, W., Oh, C. Y., Jiang, A., Nurani, A., Zhu, D. Y., Goyal, G., Gilpin, S E., et al. (2020). Human organs-on-chips as tools for repurposing approved drugs as potential influenza and COVID19 therapeutics in viral pandemics. bioRxiv.
Si, L., Prantil-Baun, R., Benam, K. H., Bai, H., Rodas, M., Burt, M., and Ingber, D. E. (2019). Discovery of influenza drug resistance mutations and host therapeutic targets using a human airway chip. bioRxiv.
Sledz, C. A., Holko, M., de Veer, M. J., Silverman, R. H., and Williams, B. R. (2003). Activation of the interferon system by short-interfering RNAs. Nat Cell Biol 5, 834-839.
Takeuchi, O., and Akira, S. (2010). Pattern recognition receptors and inflammation. Cell 140, 805-820.
Thorns, M., Buschauer, R., Ameismeier, M., Koepke, L., Denk, T., Hirschenberger, M., Kratzat, H., Hayn, M., Mackens-Kiani, T., Cheng, J., et al. (2020). Structural basis for translational shutdown and immune evasion by the Nsp1 protein of SARS-CoV-2. Science 369, 1249-1255.
Tissari, J., Siren, J., Men, S., Julkunen, I., and Matikainen, S. (2005). IFN-alpha enhances TLR3-mediated antiviral cytokine expression in human endothelial and epithelial cells by up-regulating TLR3 expression. Journal of immunology 174, 4289-4294.
Wadman, M. (2020a). Can boosting interferons, the body's frontline virus fighters, beat COVID-19?Science.
Wadman, M. (2020b). Can interferons stop COVID-19 before it takes hold?Science 369, 125-126.
Wahl, M C, Rao, S T, and Sundaralingam, M (1996). The structure of r(UUCGCG) has a 5′-UU-overhang exhibiting Hoogsteen-like trans U. U base pairs. Nat Struct Biol 3: 24-31.
Wang, P., Xu, J., Wang, Y., and Cao, X. (2017). An interferon-independent lncRNA promotes viral replication by modulating cellular metabolism. Science 358, 1051-1055.
Winkler, E S, Bailey, A L, Kafai, N M, Nair, S, McCune, B T, Yu, J, et al. (2020). SARS-CoV-2 infection of human ACE2-transgenic mice causes severe lung inflammation and impaired function. Nat Immunol 21: 1327-1335.
Zheng, J., Yong, H. Y., Panutdaporn, N., Liu, C., Tang, K., and Luo, D. (2015). High-resolution HDX-M S reveals distinct mechanisms of RNA recognition and activation by RIG-I and MDA5. Nucleic Acids Res 43, 1216-1230.
Zhou, Y., Li, M., Xue, Y., Li, Z., Wen, W., Liu, X., Ma, Y., Zhang, L., Shen, Z., and Cao, X. (2019). Interferon-inducible cytoplasmic lncLrrc55-A S promotes antiviral innate responses by strengthening IRF3 phosphorylation. Cell Res 29, 641-654.

### Example 2: Methods

Key resource table

| REAGENT OR RESOURCE | SOURCE | IDENTIFIER |
|---|---|---|
| Antibodies | | |
| anti-IRF3 | Abcam | Cat# ab68481 |
| Anit-IRF3(Phospho S396) | Abcam | Cat# ab138449 |
| Anti-GAPDH | Abcam | Cat# ab9385 |
| Goat anti-Rabbit IgG | Abcam | Cat# ab205718 |
| H&L (HRP) | | |
| Recombinant proteins | | |
| RIG-I | Abcam | Cat# ab271486 |
| MDA5 | Creative- | Cat# IFIH1-1252H |
| | Biomart | |
| TLR3 | Abcam | Cat# ab73825 |
| Experimental Models: | | |
| Cell Lines | | |
| A549 | ATCC | CCL-185 |
| A549-Dual cell | InvivoGen | |
| RIG-I KO A549-Dual ™ cell | InvivoGen | |
| MDA5 KO A549-Dual ™ cell | InvivoGen | |
| TLR3 KO A549 cell | Abcam | |
| MDCK cell | ATCC | CRL-2936 |
| LLC-MK2 cell | ATCC | CCL-7.1 |
| HAP1 cell | Horizon | |
| | Discovery | |
| | Ltd | |
| IRF3 KO HAPI cell | Horizon | |
| | Discovery | |
| | Ltd | |
| IRF7 KO HAPI cell | Horizon | |
| | Discovery | |
| | Ltd | |
| Experimental Models: | | |
| Primary cells | | |
| Primary human lung airway | Lonza | Cat# CC-2540S; |
| epithelial cells | | Lots: #448571, |
| | | #446317, #623950, |
| | | #485960, #672447, |
| | | #370751 |
| Primary human pulmonary | Lonza | #CC-2527 |
| microvascular endothelial | | |
| cells | | |
| Primary human alveolar | Cell Biologics | #H-6053 |
| epithelialcells | | |
| Experimental Models: Animal | | |
| Outbred male Syrian golden | Charles River | NA |
| hamsters, 3-5 weeks of age | Laboratories | |

### Cell Culture

A549 cells (ATCC CCL-185), A549-Dual™ cells (InvivoGen), RIG-I KO A549-Dual™ cells (InvivoGen), MDA5 KO A549-Dual™ cells (InvivoGen), TLR3 KO A549 cells (Abcam), HEK-Blue™ Null-k cells (InvivoGen, hkb-null1k), HEK-Blue™ hTLR7 cells (InvivoGen, htlr7), THP1-Dual™ cells (InvivoGen, thpd-nifs), THP1-Dual™ KO-TLR8 cells (InvivoGen,kotlr8), MDCK cells (ATCC CRL-2936), and LLC-MK2 cells (ATCC CCL-7.1) were cultured in Dulbecco's modified Eagle's medium (DMEM) (Life Technologies) supplemented with 10% fetal bovine serum (FBS) (Life Technologies) and penicillin-streptomycin (Life Technologies). HAP1 cells, IRF3 KO HAP1 cells, and IRF7 KO HAP1 cells were purchased from Horizon Discovery Ltd and cultured in Iscove's Modified Dulbecco's Medium (IMDM) (Gibco) supplemented with 10% fetal bovine serum (FBS) (Life Technologies) and penicillin-streptomycin (Life Technologies). All cells were maintained at 37° C. and 5% CO₂ in a humidified incubator. All cell lines used in this study were free of mycoplasma, as confirmed by the LookOut Mycoplasma PCR Detection Kit (Sigma). Cell lines were authenticated by the ATCC, InvivoGen, Abcam, or Horizon Discovery Ltd. Primary human lung airway epithelial basal stem cells (Lonza, USA) were expanded in 75 cm² tissue culture flasks using airway epithelial cell growth medium (Promocell, Germany) until 60-70% confluent. Primary human alveolar epithelial cells (Cell Biologics, H-6053) were cultured using alveolar epithelial growth medium (Cell Biologics, H6621). Primary human pulmonary microvascular endothelial cells (Lonza, CC-2527, P5) were expanded in 75 cm² tissue culture flasks using human endothelial cell growth medium (Lonza, CC-3202) until 70-80% confluent.

### Viruses

Viruses used in this study include SARS coronavirus-2 (SARS-CoV-2), human coronavirus HCoV-NL63, influenza A/WSN/33 (H1N1), and influenza A/Hong Kong/8/68 (H3N2). SARS-CoV-2 isolate USA-WA1/2020 (NR-52281) was deposited by the Center for Disease Control and Prevention, obtained through BEI Resources, NIAID, NIH, and propagated as described previously (Blanco-Melo et al., 2020). HCoV-NL63 was obtained from the ATCC and expanded in LLC-MK2 cells. Influenza A/WSN/33 (HIN1) was generated using reverse genetics technique and influenza A/Hong Kong/8/68 (H3N2) was obtained from the ATCC. Both influenza virus strains were expanded in MDCK cells. HCoV-NL63 was titrated in LLC-MK2 cells by Reed-Muench method. Influenza viruses were titrated by plaque formation assay (Si et al., 2020). All experiments with native SARS-CoV-2, SARS-CoV, and MERS-CoV were performed in a BSL3 laboratory and approved by our Institutional Biosafety Committee.

### Stimulation of Cell Lines by Transfection

All RNAs and negative control dsRNA were synthesized by Integrated DNA Technologies, Inc. (IDT). The poly(I:C) was purchased from InvivoGen, which specifically confirmed the absence of contamination by bacterial lipoproteins or endotoxins. 5′ triphosphate double-stranded RNA (Cat #tlrl-3pma) were purchased from Invivogen. Cells were seeded into 6-well plate at 3×10⁵ cells/well or 96-well plate at 10⁴ cells/well and cultured for 24 h before transfection. Transfection was performed using TransIT-X2 Dynamic Delivery System (Mirus) according to the manufacturer's instructions with some modifications. If not indicated otherwise, 6.8 μL of 10 μM RNA stock solution and 5 μL of transfection reagent were added in 200 μL Opti-MEM (Invitrogen) to make the transfection mixture. For transfection in 6-well plate, 200 μL of the transfection mixture was added to each well; for transfection in 96-well plate, 10 μL of the transfection mixture was added to each well. At indicated times after transfection, cell samples were collected and subjected to RNA-seq (Genewiz, Inc.), TMT Mass spectrometry, qRT-PCR, western blot, Quanti-Luc assay, and Quanti-Blue assay (InvivoGen).

### RNA-Seq and Gene Ontogeny Analysis

RNA-seq was processed by Genewiz using a standard RNA-seq package that includes polyA selection and sequencing on an Illumina HiSeq with 150-bp pair-ended reads. Sequence reads were trimmed to remove possible adapter sequences and nucleotides with poor quality using Trimmomatic v.0.36. The trimmed reads were mapped to the Homo sapiens GRCh38 reference genome using the STAR aligner v.2.5.2b. Unique gene hit counts were calculated by using feature Counts from the Subread package v.1.5.2 followed by differential expression analysis using DESeq2. Gene Ontology analysis was performed using DAVID (Huang da et al., 2009). Volcano plots and heat maps were generated using the EnhancedVolcano R package 56. Raw sequencing data files were deposited on NCBI GEO with accession number GSE124144 (Burke et al., 2019).

### Proteomics Analysis by Tandem Mass Tag Mass Spectrometry

Cells were harvested on ice. Cells pellets were syringe-lysed in 8 M urea and 200 mM EPPS pH 8.5 with protease inhibitor. BCA assay was performed to determine protein concentration of each sample. Samples were reduced in 5 mM TCEP, alkylated with 10 mM iodoacetamide, and quenched with 15 mM DTT. 100 μg protein was chloroform-methanol precipitated and re-suspended in 100 μL 200 mM EPPS pH 8.5. Protein was digested by Lys-C at a 1:100 protease-to-peptide ratio overnight at room temperature with gentle shaking. Trypsin was used for further digestion for 6 hours at 37° C. at the same ratio with Lys-C. After digestion, 30 μL acetonitrile (ACN) was added into each sample to 30% final volume. 200 μg TMT reagent (126, 127N, 127C, 128N, 128C, 129N, 129C, 130N, 130C) in 10 μL ACN was added to each sample. After 1 hour of labeling, 2 μL of each sample was combined, desalted, and analyzed using mass spectrometry. Total intensities were determined in each channel to calculate normalization factors. After quenching using 0.3% hydroxylamine, eleven samples were combined in 1:1 ratio of peptides based on normalization factors. The mixture was desalted by solid-phase extraction and fractionated with basic pH reversed phase (BPRP) high performance liquid chromatography (HPLC), collected onto a 96 six well plate and combined for 24 fractions in total. Twelve fractions were desalted and analyzed by liquid chromatography-tandem mass spectrometry (LC-MS/MS) (Navarrete-Perea et al., 2018).

Mass spectrometric data were collected on an Orbitrap Fusion Lumos mass spectrometer coupled to a Proxeon NanoLC-1200 UHPLC. The 100 μm capillary column was packed with 35 cm of Accucore 50 resin (2.6 m, 150 Å; ThermoFisher Scientific). The scan sequence began with an MS1 spectrum (Orbitrap analysis, resolution 120,000, 375-1500 Th, automatic gain control (AGC) target 4E5, maximum injection time 50 ms). SPS-MS3 analysis was used to reduce ion interference (Gygi et al., 2019; Paulo et al., 2016). The top ten precursors were then selected for MS2/MS3 analysis. MS2 analysis consisted of collision-induced dissociation (CID), quadrupole ion trap analysis, automatic gain control (AGC) 2E4, NCE (normalized collision energy) 35, q-value 0.25, maximum injection time 35 ms), and isolation window at 0.7. Following acquisition of each MS2 spectrum, an MS3 spectrum was collected in which multiple MS2 fragment ions are captured in the MS3 precursor population using isolation waveforms with multiple frequency notches. MS3 precursors were fragmented by HCD and analyzed using the Orbitrap (NCE 65, AGC 1.5E5, maximum injection time 120 ms, resolution was 50,000 at 400 Th).

Mass spectra were processed using a Sequest-based pipeline (Huttlin et al., 2010). Spectra were converted to mzXML using a modified version of ReAdW.exe. Database searching included all entries from the Human UniProt database (downloaded: 2014-02-04) This database was concatenated with one composed of all protein sequences in the reversed order. Searches were performed using a 50 ppm precursor ion tolerance for total protein level analysis. The product ion tolerance was set to 0.9 Da. TMT tags on lysine residues and peptide N termini (+229.163 Da) and carbamidomethylation of cysteine residues (+57.021 Da) were set as static modifications, while oxidation of methionine residues (+15.995 Da) was set as a variable modification.

Peptide-spectrum matches (PSMs) were adjusted to a 1% false discovery rate (FDR) (Elias and Gygi, 2007, 2010). PSM filtering was performed using a linear discriminant analysis (LDA), as described previously (Huttlin et al., 2010), while considering the following parameters: XCorr, ACn, missed cleavages, peptide length, charge state, and precursor mass accuracy. For TMT-based reporter ion quantitation, the summed signal-to-noise (S:N) ratio was extracted for each TMT channel and found the closest matching centroid to the expected mass of the TMT reporter ion. For protein-level comparisons, PSMs were identified, quantified, and collapsed to a 1% peptide false discovery rate (FDR) and then collapsed further to a final protein-level FDR of 1%, which resulted in a final peptide level FDR of <0.1%. Moreover, protein assembly was guided by principles of parsimony to produce the smallest set of proteins necessary to account for all observed peptides. Proteins were quantified by summing reporter ion counts across all matching PSMs, as described previously (Huttlin et al., 2010). PSMs with poor quality, MS3 spectra with TMT reporter summed signal-to-noise of less than 100, or having no MS3 spectra were excluded from quantification (McAlister et al., 2012). Each reporter ion channel was summed across all quantified proteins and normalized assuming equal protein loading of all tested samples.

qRT-PCR

Total RNA was extracted from cells using RNeasy Plus Mini Kit (QiaGen, Cat #74134) according to the manufacturer's instructions. cDNA was then synthesized using AMV reverse transcriptase kit (Promega) according to the manufacturer's instructions. To detect gene levels, quantitative real-time PCR was carried out using the GoTaq qPCR Master Mix kit (Promega) with 20 μL of reaction mixture containing gene-specific primers or the PrimePCR assay kit (Bio-Rad) according the manufacturers' instructions. The expression levels of target genes were normalized to GAPDH.

### Antibodies and Western Blotting

The antibodies used in this study were anti-IRF3 (Abcam, ab68481), anti-IRF3 (Phospho S396) (Abcam, ab138449), anti-GAPDH (Abcam, ab9385), and Goat anti-Rabbit IgG H&L (HRP) (Abcam, ab205718). Cells were harvested and lysed in RIPA buffer (Thermo Scientific, Cat #89900) supplemented with Halt™ protease and phosphatase inhibitor cocktail (Thermo Scientific, Cat #78440) on ice. The cell lysates were subject to western blotting. GAPDH was used as a loading control.

### Confocal Immunofluorescence Microscopy

Cells were rinsed with PBS, fixed with 4% paraformaldehyde (Alfa Aesar) for 30 min, permeabilized with 0.1% Triton X-100 (Sigma-Aldrich) in PBS (PBST) for 10 min, blocked with 10% goat serum (Life Technologies) in PBST for 1 h at room temperature, and incubated with anti-IRF3 (Phospho S396) (Abcam, ab138449) antibody diluted in blocking buffer (1% goat serum in PBST) overnight at 4° C., followed by incubation with Alexa Fluor 488 conjugated secondary antibody (Life Technologies) for 1 h at room temperature; nuclei were stained with DAPI (Invitrogen) after secondary antibody staining. Fluorescence imaging was carried out using a confocal laser-scanning microscope (SP5×MP DMI-6000, Germany) and image processing was done using Imaris software (Bitplane, Switzerland).

### Surface Plasmon Resonance

The interactions between duplex RNA-1 and cellular RNA sensor molecules (RIG-I (Abcam, Cat #ab271486), MDA5 (Creative-Biomart, Cat #IFIH1-1252H), and TLR3 (Abcam, Cat #ab73825)) were analyzed by SPR with the Biacore T200 system (GE Healthcare) at 25° C. (Creative-Biolabs Inc.). RNA-1 conjugated with biotin at 3′ end of sense strand (synthesized by IDT Inc.) was immobilized on a SPR Series S Sensor Chip SA (GE Healthcare, Cat #BR100531) by flowing 2 nM RNA-1 conjugated with biotin diluted in running buffer (10×HBS-EP+; GE Healthcare, Cat #BR100669) on the surface of SPR chip, with final levels of ˜50 response units (RU). Indicated concentrations of the RNA sensors (RIG-I, MDA5, or TLR3) diluted in running buffer (10×HBS-EP+; GE Healthcare, Cat #BR100669) were injected as analytes at a flow rate of 30 l/min, a contact time of 180 s, and a dissociation time of 420 s. The surface was regenerated with 2 M NaCl for 30 s. Data analysis was performed on the Biacore T200 computer with the Biacore T200 evaluation software.

### Organ Chip Culture

Microfluidic two-channel Organ Chip devices and automated ZOE® instruments used to culture them were obtained from Emulate Inc (Boston, MA, USA). Methods methods for culturing human Lung Airway Chips (Si et al., 2020; Si et al., 2019) and Lung Alveolus Chips have been described previously. In the study described herein, the Alveolus Chip method was slightly modified by coating the inner channels of the devices with 200 μg/ml Collagen IV (5022-5MG, Advanced Biomatrix) and 15 g/ml of laminin (L4544-100UL, Sigma) at 37° C. overnight, and the next day (day 1) sequentially seeding primary human lung microvascular endothelial cells (Lonza, CC-2527, P5) and primary human lung alveolar epithelial cells (Cell Biologics, H-6053) in the bottom and top channels of the chip at a density of 8 and 1.6×10⁶ cells/ml, respectively, under static conditions. On day 2, the chips were inserted into Pods® (Emulate Inc.), placed within the ZOE® instrument, and the apical and basal channels were respectively perfused (60 L/hr) with epithelial growth medium (Cell Biologics, H6621) and endothelial growth medium (Lonza, CC-3202). On day 5, 1 uM dexamethasone was added to the apical medium to enhance barrier function. On day 7, an air-liquid interface (ALI) was introduced into the epithelial channel by removing all medium from this channel while continuing to feed all cells through the medium perfused through the lower vascular channel, and this medium was changed to EGM-2MV with 0.5% FBS on day 9. Two days later, the ZOE® instrument was used to apply cyclic (0.25 Hz) 5% mechanical strain to the engineered alveolar-capillary interface to mimic lung breathing on-chip. RNAs were transfected on Day 15.

RNA Transfection in Human LungAirway andAlveolus Chips

Human Airway or Alveolus Chips were transfected with duplex RNAs by adding the RNA and transfection reagent (Lipofectamine RNAiMAX) mixture into the apical and basal channels of the Organ Chips and incubating for 6 h at 37° C. under static conditions before reestablishing an ALI. Tissues cultured on-chip were collected by RNeasy Micro Kit (QiaGen) at 48 h post-transfection by first introducing 100 ul lysis buffer into the apical channel to lyse epithelial cells and then 100 ul into the basal channel to lyse endothelial cells. Lysates were subjected to qPCR analysis of IFN-β gene expression.

### Native SARS-CoV-2 Infection and Inhibition by RNA Treatment

ACE2-expressing A549 cells (a gift from Brad Rosenberg) were transfected with indicated RNAs. 24 h post-transfection, the transfected ACE2-A549 cells were infected with SARS-CoV-2 (MOI=0.05) for 48 hours. Cells were harvested in Trizol (Invitrogen) and total RNA was isolated and DNAse-I treated using Zymo RNA Miniprep Kit according to the manufacturer's protocol. qRT-PCR for α-tubulin (Forward: 5′-GCCTGGACCACAAGTTTGAC-3′ (SEQ ID NO: 31); Reverse: 3′-TGAAATTCTGGGAGCATGAC-5′) (SEQ ID NO: 32) and SARS-CoV-2 N mRNA (Forward: 5′-CTCTTGTAGATCTGTTCTCTAAACGAAC-3′ (SEQ ID NO: 33); Reverse: 3′-GGTCCACCAAACGTAATGCG-5′) (SEQ ID NO: 34) were performed using KAPA SYBR FAST ONE-STEP qRT-PCR kits (Roche) according to manufacturer's instructions on a Lightcycler 480 Instrument-II (Roche).

### Native SARS-CoV-I and MERS-CoV Infection and Inhibition by RNA Treatment

Vero E6 cells (ATCC #CRL 1586) were cultured in DMEM (Quality Biological), supplemented with 10% (v/v) fetal bovine serum (Sigma), 1% (v/v) penicillin/streptomycin (Gemini Bio-products) and 1% (v/v) L-glutamine (2 mM final concentration, Gibco). Cells were maintained at 37° C. (5% CO₂). Vero E6 cells were plated at 1.5×10⁵ cells per well in a six well plate two days prior to transfection. The RNA-1, RNA-2, and scrambled control RNA were transfected into each well using the Transit X2 delivery system (MIRUS; MIR6003) in OptiMEM (Gibco 31985-070). SARS-CoV (Urbani strain, BEI #NR-18925) and MERS-CoV (Jordan strain, provided by NIH) were added at MOI 0.01. At 72 hours post infection, medium was collected and used for a plaque assay to quantify PFU/mL of virus.

### Hamster Efficacy Studies

The efficacy studies were carried out in Golden hamsters using native SARS-CoV-2 Isolate USA-WA1/2020 (NR-52281), as described previously (13). In the prevention studies, RNA-1 diluted in 5% glucose containing in vivo-jetPEI® Delivery Reagent (Genesee Scientific Cat #: 55-202G; 20 μg in 50 uL) was administered intranasally beginning 1 day prior to intranasal administration of SARS-CoV-2 virus (10² PFU of passage 3 virus in 100 μl of PBS) and daily for 2 additional days. In the treatment experiments, RNA-1 diluted in 5% glucose containing in vivo-jetPEI® Delivery Reagent (Genesee Scientific Cat #: 55-202G; 20 μg in 50 uL) was administered intranasally daily for 2 days beginning 1 day after intranasal administration of SARS-CoV-2 virus (10³ PFU). In all experiments, animals were sacrificed and lungs harvested for analysis 1 day after the last treatment was administered. Animals were anesthetized by intraperitoneal injection of 100 μl of ketamine and xylazine (3:1) and provided thermal support while unconscious, and whole lungs were harvested for analysis by RT-qPCR or plaque assay.

Lung RNA was extracted by phenol chloroform extraction and DNase treatment using DNA-free DNA removal kit (Invitrogen), and RT-qPCR was performed using KAPA SYBR FAST qPCR Master Mix Kit (Kapa Biosystems) on a LightCycler 480 Instrument II (Roche) for subgenomic nucleocapsid (N) RNA (sgRNA) and actin using the following primers: Actin forward primer: 5′-CCAAGGCCAACCGTGAAAAG-3′ (SEQ ID NO: 35), Actin reverse primer 5′-ATGGCTACGTACATGGCTGG-3′ (SEQ ID NO: 36), N sgRNA forward primer: 5′-CTCTTGTAGATCTGTTCTCTAAACGAAC-3′ (SEQ ID NO: 37), N sgRNA reverse primer: 5′-GGTCCACCAAACGTAATGCG-3′ (SEQ ID NO: 38). Relative sgRNA levels were quantified by normalizing sgRNA to actin expression.

All experiments with native SARS-CoV-2, SARS-CoV, and MERS-CoV were performed in a BSL3 laboratory and approved by the inventors' Institutional Biosafety Committee.

### Efficacy Study in K18-hACE2 Mice

Thirty-two, six-week-old, female K18-hACE2 mice were purchased from Jackson Laboratory. Mice were randomized into four groups (n=4) with vehicle, and vehicle plus treatment; 15 μg RNA-1, 45 μg RNA-1 or 45 μg negative control RNA. Vehicle or treatment were delivered by intravenous administration through the tail in a total volume of 100 μl. Treatment was conducted at −24 hr and −2 hr prior to viral infection (two doses in total). Mice were weighed daily and physically assessed for signs of morbidity, anesthetized with isoflurane and intranasally challenged with 2×10⁴ pfu per mouse (25 μl/naris) using the SARS-CoV-2 WA1 strain (20×LD50). All animals were sacrificed in each group on 3 days post infection, and the lungs were collected for analysis. The left lobe of the lung tissue was placed into a bead mill tube (1.4 mm ceramic beads) containing a 1 mL solution of protease inhibitors (Halt Protease Inhibitor Cocktail) in PBS, homogenized using the Bead Mill 4 (Fisher) for 1-2 cycles of 10 sec (5 m/s), centrifuged at 16,000×g and the supernatant was aliquoted and flash-frozen in liquid nitrogen before being placed in a −80° C. freezer. The viral titers were determined using plaque assay as described previously.

### Quantification and Statistical Analysis

All data are expressed as mean±standard deviation (SD). N represents biological replicates. Statistical significance of differences in the in vitro experiments was determined by employing the paired two-tailed Student t-test when comparing the difference between two groups and one-way ANOVA with multiple comparison when comparing the samples among groups with more than two samples. For in vivo experiments, an unpaired one-tailed Student t-test was used to estimate significance of viral load inhibition by RNA-1. For all experiments, differences were considered statistically significant for p<0.05 (*, p<0.05; **, p<0.01; ***, p<0.001; n.s., not significant).

1. An immunostimulatory complex comprising a concatamer of oligonucleotide duplexes, wherein each duplex comprises an oligonucleotide strand having the structure 5′-C—N₁₆-GGG-3′ and an oligonucleotide strand having the structure 5′-C—N′₁₆-GGG-3′, wherein:
N and N′ are each any of G, A, U and C;
N₁₆ is complementary to N′₁₆; and
duplexes in the concatamer are joined by Hoogsteen base pairing between 3′-GG overhanging dinucleotides on each duplex.

2. The immunostimulatory complex of claim 1, wherein the concatamer is a dimer of oligonucleotide duplexes.

3. The immunostimulatory complex of claim 1, wherein the concatamer comprises three or more of the oligonucleotide duplexes.

4. The immunostimulatory complex of any one of claims 1-3, wherein one or both oligonucleotide strands of each duplex comprise(s) a 5′-terminal monophosphate, diphosphate, triphosphate or hydroxyl group.

5. The immunostimulatory complex of any one of claims 1-4, wherein the oligonucleotide duplexes comprise double stranded RNA.

6. The immunostimulatory complex of any one of claims 1-5, wherein the concatamer induces interferon (IFN) production in a cell.

7. The immunostimulatory complex of claim 6, wherein the IFN production is type I IFN production.

8. The immunostimulatory complex of any one of claims 1-7, wherein the concatamer activates the RIG-I-IRF3 pathway.

9. The immunostimulatory complex of any one of claims 1-8, wherein the concatamer reduces a viral titer or viral load in a cell or population of cells.

10. An immunostimulatory complex comprising at least first and second RNA duplexes, each duplex comprising:
a first strand comprising, from the 5′ terminus, the sequence 5′-C—N₁₉-3′, and
a second strand comprising, at the 3′ terminus, the sequence 5′-N′₁₉-GGG-3′,
wherein:
N and N′ are any of C, A, G, and U;
N and N′ are complementary;
the 3′ terminal GG dinucleotide of the second strand forms a 3′ GG dinucleotide overhang;
the first duplex is complexed with the at least second duplex via Hoogsteen base pairing between the 3′ GG overhang on each duplex; and
the first strand, at the 5′ terminus, does not comprise the sequence 5′-CUGA-3′.

11. The immunostimulatory complex of claim 10, wherein one or both oligonucleotide strands of each duplex comprise(s) a 5′-terminal monophosphate, diphosphate, triphosphate or hydroxyl group.

12. The immunostimulatory complex of claim 10 or 11, wherein the RNA duplexes comprise double stranded RNA.

13. The immunostimulatory complex of any one of claims 10-12, wherein the RNA duplexes comprise one or more DNA nucleotides at the duplex end opposite the 5′-C.

14. The immunostimulatory complex of any one of claims 10-13, wherein the RNA duplexes comprise comprises a blunt end, a 5′ overhang or a 3′ overhang on the end opposite the 5′-C.

15. The immunostimulatory complex of any one of claims 10-14, wherein the complex induces interferon (IFN) production in a cell.

16. The immunostimulatory complex of claim 15, wherein the IFN production is type I IFN production.

17. The immunostimulatory complex of any one of claims 10-16, wherein the complex activates the RIG-I-IRF3 pathway.

18. The immunostimulatory complex of any one of claims 10-17, wherein the complex reduces a viral titer or viral load in a cell or population of cells.

19. A pharmaceutical composition comprising the immunostimulatory complex of any one of claims 1-18.

20. The pharmaceutical composition of claim 19, which further comprises a pharmaceutically acceptable carrier.

21. The composition of claim 19 or 20, wherein the composition is formulated for airway administration.

22. The composition of any one of claims 19-21, wherein the composition is formulated for aerosol administration, nebulizer administration, or tracheal lavage administration.

23. A composition comprising an immunostimulatory complex of any one of claims 1-18 or a pharmaceutical composition of any one of claims 19-22 and a vaccine.

24. A composition comprising an immunostimulatory complex of any one of claims 1-18 or a pharmaceutical composition of any one of claims 19-22 and a nanoparticle.

25. A nanoparticle comprising an immunostimulatory complex of any one of claims 1-18 or a pharmaceutical composition of any one of claims 19-22.

26. A method of inducing an anti-viral response in a subject, the method comprising administering to a subject in need thereof an immunostimulatory complex of any one of claims 1-18 or a pharmaceutical composition of any one of claims 19-25.

27. A method of treating or preventing a viral infection in a subject, the method comprising administering to a subject in need thereof an immunostimulatory complex of any one of claims 1-18 or a pharmaceutical composition of any one of claims 19-25.

28. The method of claim 26 or 27, wherein the subject in need thereof has a viral infection, or is at risk of having a viral infection.

29. The method of any one of claims 26-28, further comprising, prior to administering, a step of diagnosing the subject as having a viral infection or being at risk of having a viral infection.

30. The method of any one of claims 26-28, further comprising, prior to administering, a step of receiving results of an assay that diagnoses the subject as having a viral infection or as being at risk of having a viral infection.

31. The method of any one of claims 27-30, wherein the viral infection is caused by a virus selected from the group consisting of: John Cunningham virus, measles virus, Lymphocytic choriomeningitis virus, arbovirus, rabies virus, rhinovirus, parainfluenza virus, respiratory syncytial virus, herpes simplex virus, herpes simplex type 1, herpes simplex type 2, human herpesvirus 6, adenovirus, cytomegalovirus, Epstein-Barr virus, mumps virus, influenza virus type A, influenza virus type B, coronavirus, SARS coronavirus, SARS-CoV-2 virus, coxsackie A virus, coxsackie B virus, poliovirus, HTLV-1, hepatitis virus types A, B, C, D, and E, varicella zoster virus, smallpox virus, molluscum contagiosum, human papillomavirus, parvovirus B19, rubella virus, human immunodeficiency virus, rotavirus, norovirus, astrovirus, ebola virus, Marburg virus, dengue virus (DENV), and Zika virus.

32. The method of any one of claims 27-31, wherein the viral infection is an infection of a tissue selected from the group consisting of central nervous system tissue, eye tissue, upper respiratory system tissue, lower respiratory system tissue, lung tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.

33. The method of any one of claims 26-32, wherein the administration is systemic.

34. The method of any one of claims 26-32, wherein the administration is local at a site of viral infection.

35. The method of any one of claims 26-34, further comprising administering at least one additional therapeutic.

36. The method of claim 35, wherein the at least one additional therapeutic is an anti-viral therapeutic.

37. A method of treating an influenza infection in a subject, the method comprising administering to a subject having an influenza infection an immunostimulatory complex of any one of claims 1-18 or a pharmaceutical composition of any one of claims 19-25.

38. The method of claim 37, wherein the influenza infection is an influenza A infection, or an influenza B infection.

39. The method of claim 37 or 38, further comprising administering at least one additional anti-viral therapeutic.

40. A method of treating a coronavirus disease in a subject, the method comprising administering to a subject having a coronavirus infection an immunostimulatory complex of any one of claims 1-18 or a pharmaceutical composition of any one of claims 19-25.

41. The method of claim 40, wherein the coronavirus disease is COVID-19.

42. The method of claim 40 or 41, further comprising administering at least one additional anti-viral therapeutic.

43. A method of inducing interferon (IFN) production, the method comprising administering to a subject in need thereof an immunostimulatory complex of any one of claims 1-18 or a pharmaceutical composition of any one of claims 19-25, whereby IFN production is increased following administration.

44. The method of claim 43, wherein IFN production is the production of type I IFN, type II IFN, or type III IFN.

45. The method of claim 43 or 44, wherein IFN production is the production of type I IFN.

46. An immunostimulatory RNA duplex having
a) a first strand having from 5′ to 3′ a GNNN (SEQ ID NO: 1) sequence flanked by at least 22 nucleotides on each side; and
b) a second strand having from 5′-3′ a GGGC (SEQ ID NO: 2) sequence flanked by at least 22 nucleotides on each side,
wherein the first and second strands are complementary to each other.

47. The RNA duplex of claim 46, wherein the first and/or second strand has a two nucleotide overhang at its 3′ end.

48. The RNA duplex of claim 46 or 47, wherein the first and/or second strand have two DNA nucleosides at its 3′ end.

49. The RNA duplex of claim 48, wherein the DNA nucleosides are thymidines.

50. The RNA duplex of any one of claims 46-49, wherein the first and/or second strand has a TT overhang at its 3′ end.

51. The RNA duplex of any one of claims 46-50, wherein the first and/or second strand comprises a 5′-terminal monophosphate, diphosphate, triphosphate or hydroxyl group.

52. The RNA duplex of any of claims 46-51, wherein the RNA duplex is synthetic.

53. The RNA duplex of any of claims 46-52, wherein the RNA duplex induces interferon (IFN) production in a cell.

54. The RNA duplex of claim 53, wherein the IFN production is type I IFN production.

55. The RNA duplex of any one of claims 46-54, wherein the RNA duplex activates the RIG-I-IRF3 pathway.

56. The RNA duplex of any one of claims 46-55, wherein the RNA duplex reduces a viral titer or viral load in a cell or population of cells.

57. A synthetic RNA duplex having a first and second strand having a sequence selected from SEQ ID NO: 7-32.

58. A method of inducing an anti-viral response is a subject, the method comprising administering to a subject in need thereof an RNA duplex of any of claims 46-56, or a synthetic RNA duplex of claim 57.

59. A method of treating a viral infection in a subject, the method comprising administering to a subject in need thereof an RNA duplex of any of claims 46-56, or a synthetic RNA duplex of claim 57.

60. The method of claim 58 or 59, wherein the subject in need thereof has a viral infection, or is at risk of having a viral infection.

61. The method of claim 58 or 59, further comprising, prior to administering, a step of diagnosing the subject as having a viral infection or being at risk of having a viral infection.

62. The method of claim 58 or 59, further comprising, prior to administering, a step of receiving results of an assay that diagnoses the subject as having a viral infection or as being at risk of having a viral infection.

63. The method of any one of claims 59-62, wherein the viral infection is caused by a virus selected from the group consisting of: John Cunningham virus, measles virus, Lymphocytic choriomeningitis virus, arbovirus, rabies virus, rhinovirus, parainfluenza virus, respiratory syncytial virus, herpes simplex virus, herpes simplex type 1, herpes simplex type 2, human herpesvirus 6, adenovirus, cytomegalovirus, Epstein-Barr virus, mumps virus, influenza virus type A, influenza virus type B, coronavirus, SARS coronavirus, SARS-CoV-2 virus, coxsackie A virus, coxsackie B virus, poliovirus, HTLV-1, hepatitis virus types A, B, C, D, and E, varicella zoster virus, smallpox virus, molluscum contagiosum, human papillomavirus, parvovirus B19, rubella virus, human immunodeficiency virus, rotavirus, norovirus, astrovirus, ebola virus, Marburg virus, dengue virus (DENV), and Zika virus.

64. The method of any one of claims 59-63, wherein the viral infection is an infection of a tissue selected from the group consisting of central nervous system tissue, eye tissue, upper respiratory system tissue, lower respiratory system tissue, lung tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.

65. The method of any one of claims 58-64, wherein the administration is systemic.

66. The method of any one of claims 58-64, wherein the administration is local at a site of viral infection.

67. The method of any of claims 58-66, further comprising administering at least one additional therapeutic.

68. The method of claim 67, wherein the at least one additional therapeutic is an anti-viral therapeutic.

69. A method of treating an influenza infection in a subject, the method comprising administering to a subject having an influenza infection an RNA duplex of any of claims 46-56, or a synthetic RNA duplex of claim 57.

70. The method of claim 69, wherein the influenza infection is an influenza A infection, or an influenza B infection.

71. The method of claim 69 or 70, further comprising administering at least one additional anti-viral therapeutic.

72. A method of treating a coronavirus disease in a subject, the method comprising administering to a subject having a coronavirus disease an RNA duplex of any of claims 46-56, or a synthetic RNA duplex of claim 57.

73. The method of claim 72, wherein the coronavirus disease is COVID-19.

74. The method of claim 72 or 73, further comprising administering at least one additional anti-viral therapeutic.

75. A method of increasing the efficacy of an anti-viral therapeutic, the method comprising administering an RNA duplex of any of claims 46-56, or a synthetic RNA duplex of claim 57 and at least one anti-viral therapeutic.

76. The method of claim 75, wherein the anti-viral therapeutic is selected from the group consisting of: Abacavir, Acyclovir (Aciclovir), Adefovir, Amantadine, Ampligen, Amprenavir (Agenerase), Amodiaquine, Apilimod, Arbidol, Atazanavir, Atripla, Atovaquone, Balavir, Baloxavir marboxil (Xofluza®), Biktarvy Boceprevir (Victrelis®), Cidofovir, Clofazimine, Clomifene, Clofazamine, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Favipiravir, Fenofibrate, Fomivirsen, Fosamprenavir, Foscarnet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Ivermectin, Lamivudine, Lasalocid, Letermovir (Prevymis®), Lopinavir, Loviride, Mannose Binding Lectin, Maraviroc, Methisazone, Moroxydine, Nafamostat, Nelfinavir, Nevirapine, Nexavir®, Nilotinib, Nitazoxanide, Norvir, Nucleoside analogues, Oseltamivir (Tamiflu®), Pazopanib, Peginterferon alfa-2a, Peginterferon alfa-2b, Penciclovir, Peramivir (Rapivab®), Pleconaril, Podophyllotoxin, Protease inhibitor (pharmacology), Pyonaridine, Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Tafenoquine, Telaprevir, Telbivudine (Tyzeka®), Tenofovir alafenamide, Tenofovir disoproxil, Tenofovir, Toremifene, Tipranavir, Trifluridine, Trizivir, Tromantadine, Truvada, Valaciclovir (Valtrex), Valganciclovir, Vermurafenib, Venetoclax, Vicriviroc, Vidarabine, Viramidine, Zalcitabine, Zanamivir (Relenza®), and Zidovudine.

77. The method of claim 75 or 76, wherein the RNA duplex and the at least one antiviral therapeutic are administered at substantially the same time.

78. The method of claim 75 or 76, wherein the RNA duplex and the at least one antiviral therapeutic are administered at different time points.

79. A pharmaceutical composition comprising an RNA duplex of any one of claims 46-56, or a synthetic RNA duplex of claim 57 and a pharmaceutically acceptable carrier.

80. A pharmaceutical composition comprising an RNA duplex of any one of claims 46-56, or a synthetic RNA duplex of claim 57 and at least one anti-viral therapeutic.

81. The composition of claim 79 or 80, wherein the composition is formulated for airway administration.

82. The composition of claim 81, wherein the composition is formulated for aerosol administration, nebulizer administration, or tracheal lavage administration.

83. A method of inducing interferon (IFN) production, the method comprising administering to a subject in need thereof an RNA duplex of any of claims 46-56, a synthetic RNA duplex of claim 57, or a pharmaceutical composition of any of claims 79-82, whereby IFN production is increased following administration.

84. The method of claim 83, wherein IFN production is the production of type I IFN, type II IFN, or type III IFN.

85. The method of claim 83 or 84, wherein IFN production is the production of type I IFN.

86. The method of any one of claims 83-85, wherein the type I IFN is IFN-α, IFN-β, IFN-ε, IFN-κ or IFN-ω.

87. The method of any one of claims 83-86, wherein increased IFN production increases cellular resistance to a viral infection.

88. A composition comprising an RNA duplex of any of claims 46-56, or a synthetic RNA duplex of claim 57 and a vaccine.

89. A composition comprising an RNA duplex of any of claims 46-56, or a synthetic RNA duplex of claim 57 and a nanoparticle.

90. A method of vaccinating, the method comprising administering to a subject in need thereof a composition of any one of claims 23-25, 88 or 89.

91. A method of increasing the efficacy of a vaccine, the method comprising administering to a subject in need thereof an immunostimulatory complex of any one of claims 1-18, a composition of any one of claims 19-25, an RNA duplex of any one of claims 46-57, or a composition of any one of claims 79-82, 88 and 89.

92. A method of preparing an RNAi molecule to promote degradation of a target RNA, the method comprising:
a) identifying CCC trinucleotide repeats in the sequence of a target RNA;
b) selecting a nucleotide sequence from 20 nucleotides to the upper limit for a dsRNA duplex that avoids a double-stranded RNA-activated protein kinase response, and lacks CCC repeats in a target RNA sequence as a candidate RNAi sequence);
c) synthesizing an RNA molecule complementary to the sequence selected in step (b); and
d) synthesizing an RNA molecule complementary to the RNA molecule synthesized in step (c),
wherein combination of the RNA molecules synthesized in steps (c) and (d) produces an RNAi molecule that is less immunostimulatory than an RNAi molecule that targets the same target RNA but comprises a CCC trinucleotide repeat.

93. The method of claim 92, wherein the nucleotide sequence of b) is 20-29 nucleotides nucleotides in length.


SEQUENCE LISTING (38 sequences)

SEQ ID NO: 1-4 - skipped (4 entries)

SEQ ID NO: 5 - RNA, 52 nt, synthetic construct
  source 1..52: mol_type = other RNA
  modified_base 51: mod_base = OTHER; note = Thymine
  modified_base 52: mod_base = OTHER; note = Thymine
aaggtgaactagccagtgtcatcagggctgatgacactggctagttcacctt

SEQ ID NO: 6 - RNA, 52 nt, synthetic construct; identical to SEQ ID NO: 5
  source 1..52: mol_type = other RNA
  modified_base 51: mod_base = OTHER; note = Thymine
  modified_base 52: mod_base = OTHER; note = Thymine

SEQ ID NO: 7 - RNA, 52 nt, synthetic construct
  source 1..52: mol_type = other RNA
  modified_base 51: mod_base = OTHER; note = Thymine
  modified_base 52: mod_base = OTHER; note = Thymine
aaggtgaactagccagtgtcatcagccctgatgacactggctagttcacctt

SEQ ID NO: 8 - RNA, 52 nt, synthetic construct; identical to SEQ ID NO: 5
  source 1..52: mol_type = other RNA
  modified_base 51: mod_base = OTHER; note = Thymine
  modified_base 52: mod_base = OTHER; note = Thymine

SEQ ID NO: 9 - RNA, 52 nt, synthetic construct; identical to SEQ ID NO: 7
  source 1..52: mol_type = other RNA

SEQ ID NO: 10 - RNA, 52 nt, synthetic construct; identical to SEQ ID NO: 5
  source 1..52: mol_type = other RNA

SEQ ID NO: 11 - RNA, 52 nt, synthetic construct; identical to SEQ ID NO: 7
  source 1..52: mol_type = other RNA
  modified_base 51: mod_base = OTHER; note = Thymine
  modified_base 52: mod_base = OTHER; note = Thymine

SEQ ID NO: 12 - RNA, 52 nt, synthetic construct; identical to SEQ ID NO: 5
  source 1..52: mol_type = other RNA
  modified_base 51: mod_base = OTHER; note = Thymine
  modified_base 52: mod_base = OTHER; note = Thymine

SEQ ID NO: 13 - RNA, 50 nt, synthetic construct
  source 1..50: mol_type = other RNA
  modified_base 49: mod_base = OTHER; note = Thymine
  modified_base 50: mod_base = OTHER; note = Thymine
ggtgaactagccagtgtcatcagccctgatgacactggctagttcacctt

SEQ ID NO: 14 - RNA, 50 nt, synthetic construct
  source 1..50: mol_type = other RNA
  modified_base 49: mod_base = OTHER; note = Thymine
  modified_base 50: mod_base = OTHER; note = Thymine
ggtgaactagccagtgtcatcagggctgatgacactggctagttcacctt

SEQ ID NO: 15 - RNA, 50 nt, synthetic construct
  source 1..50: mol_type = other RNA
aaggtgaactagccagtgtcatcagccctgatgacactggctagttcacc

SEQ ID NO: 16 - RNA, 50 nt, synthetic construct
  source 1..50: mol_type = other RNA
aaggtgaactagccagtgtcatcagggctgatgacactggctagttcacc

SEQ ID NO: 17 - RNA, 48 nt, synthetic construct
  source 1..48: mol_type = other RNA
  modified_base 47: mod_base = OTHER; note = Thymine
  modified_base 48: mod_base = OTHER; note = Thymine
aaggtgaactagccagtgtcatcatgatgacactggctagttcacctt

SEQ ID NO: 18 - RNA, 48 nt, synthetic construct; identical to SEQ ID NO: 17
  source 1..48: mol_type = other RNA
  modified_base 47: mod_base = OTHER; note = Thymine
  modified_base 48: mod_base = OTHER; note = Thymine

SEQ ID NO: 19 - RNA, 44 nt, synthetic construct
  source 1..44: mol_type = other RNA
  modified_base 43: mod_base = OTHER; note = Thymine
  modified_base 44: mod_base = OTHER; note = Thymine
aaggtgaactagccagtgtcatatgacactggctagttcacctt

SEQ ID NO: 20 - RNA, 44 nt, synthetic construct; identical to SEQ ID NO: 19
  source 1..44: mol_type = other RNA
  modified_base 43: mod_base = OTHER; note = Thymine
  modified_base 44: mod_base = OTHER; note = Thymine

SEQ ID NO: 21 - RNA, 42 nt, synthetic construct
  source 1..42: mol_type = other RNA
  modified_base 41: mod_base = OTHER; note = Thymine
  modified_base 42: mod_base = OTHER; note = Thymine
aaggtgaactagccagtgtcatgacactggctagttcacctt

SEQ ID NO: 22 - RNA, 42 nt, synthetic construct; identical to SEQ ID NO: 21
  source 1..42: mol_type = other RNA
  modified_base 41: mod_base = OTHER; note = Thymine
  modified_base 42: mod_base = OTHER; note = Thymine

SEQ ID NO: 23 - RNA, 40 nt, synthetic construct
  source 1..40: mol_type = other RNA
  modified_base 39: mod_base = OTHER; note = Thymine
  modified_base 40: mod_base = OTHER; note = Thymine
aaggtgaactagccagtgtcgacactggctagttcacctt

SEQ ID NO: 24 - RNA, 40 nt, synthetic construct; identical to SEQ ID NO: 23
  source 1..40: mol_type = other RNA
  modified_base 39: mod_base = OTHER; note = Thymine
  modified_base 40: mod_base = OTHER; note = Thymine

SEQ ID NO: 25 - RNA, 38 nt, synthetic construct
  source 1..38: mol_type = other RNA
  modified_base 37: mod_base = OTHER; note = Thymine
  modified_base 38: mod_base = OTHER; note = Thymine
aaggtgaactagccagtgtacactggctagttcacctt

SEQ ID NO: 26 - RNA, 38 nt, synthetic construct; identical to SEQ ID NO: 25
  source 1..38: mol_type = other RNA
  modified_base 37: mod_base = OTHER; note = Thymine
  modified_base 38: mod_base = OTHER; note = Thymine

SEQ ID NO: 27 - RNA, 36 nt, synthetic construct
  source 1..36: mol_type = other RNA
  modified_base 35: mod_base = OTHER; note = Thymine
  modified_base 36: mod_base = OTHER; note = Thymine
aaggtgaactagccagtgcactggctagttcacctt

SEQ ID NO: 28 - RNA, 36 nt, synthetic construct; identical to SEQ ID NO: 27
  source 1..36: mol_type = other RNA
  modified_base 35: mod_base = OTHER; note = Thymine
  modified_base 36: mod_base = OTHER; note = Thymine

SEQ ID NO: 29 - RNA, 34 nt, synthetic construct
  source 1..34: mol_type = other RNA
  modified_base 33: mod_base = OTHER; note = Thymine
  modified_base 34: mod_base = OTHER; note = Thymine
aaggtgaactagccagtactggctagttcacctt

SEQ ID NO: 30 - RNA, 34 nt, synthetic construct; identical to SEQ ID NO: 29
  source 1..34: mol_type = other RNA
  modified_base 33: mod_base = OTHER; note = Thymine
  modified_base 34: mod_base = OTHER; note = Thymine

SEQ ID NO: 31 - DNA, 20 bp, synthetic construct
  source 1..20: mol_type = other DNA
gcctggaccacaagtttgac

SEQ ID NO: 32 - DNA, 20 bp, synthetic construct
  source 1..20: mol_type = other DNA
cagtacgagggtcttaaagt

SEQ ID NO: 33 - DNA, 28 bp, synthetic construct
  source 1..28: mol_type = other DNA
ctcttgtagatctgttctctaaacgaac

SEQ ID NO: 34 - DNA, 20 bp, synthetic construct
  source 1..20: mol_type = other DNA
gcgtaatgcaaaccacctgg

SEQ ID NO: 35 - DNA, 20 bp, synthetic construct
  source 1..20: mol_type = other DNA
ccaaggccaaccgtgaaaag

SEQ ID NO: 36 - DNA, 20 bp, synthetic construct
  source 1..20: mol_type = other DNA
atggctacgtacatggctgg

SEQ ID NO: 37 - DNA, 28 bp, synthetic construct
  source 1..28: mol_type = other DNA
ctcttgtagatctgttctctaaacgaac

SEQ ID NO: 38 - DNA, 20 bp, synthetic construct
  source 1..20: mol_type = other DNA
ggtccaccaaacgtaatgcg